(19) United States
(12) Patent Application Publication
(10) Pub. No.: US 2020/0131227 A1
(43) Pub. Date: Apr. 30, 2020

(54) POLYPEPTIDE WITH TUMOUR BINDING ACTIVITY

(71) Applicant: RUPRECHT-KARLS-UNIVERSITÄT HEIDELBERG, Heidelberg (DE)

(72) Inventors: Uwe HABERKORN, Schwetzingen (DE); Walter MIER, Bensheim (DE); Max SAUTER, Heidelberg (DE); Annette ALTMANN, Schwetzingen (DE); Christel HEROLD-MENDE, Bammental (DE); Rolf WARTA, Speyer (DE); Saskia RÖSCH, Mannheim (DE)

(21) Appl. No.: 16/489,261

(22) PCT Filed: Feb. 27, 2018
(86) PCT No.: PCT/EP2018/054852

§ 371 Date: Aug. 27, 2019

(30) Foreign Application Priority Data
Feb. 27, 2017 (EP) 17158242.2

(51) Int. Cl. C07K 7/06; C07K 14/705; C07K 7/08

(52) U.S. Cl. CPC C07K 7/06; A61K 38/00; C07K 7/08; C07K 14/70546

(57) ABSTRACT

The present invention relates to a polypeptide with tumor binding activity, and its use in the treatment and diagnosis of cancer.

The present invention relates to a polypeptide with tumor binding activity, and its use in the treatment and diagnosis of cancer.

### BACKGROUND OF THE INVENTION

Radiolabeled polypeptides for diagnosis and targeted therapy of tumors are characterized by efficient transport to the tumor cells and a fast clearance. Since linear peptides display poor in vivo stability peptides embedded in disulfide-stabilized miniproteins which are endowed with an excellent proteolytic stability and beneficial pharmacokinetic profile are of growing interest for the development of tumor-affine ligands.

Integrins (ITG) are a family of heterodimeric cell surface receptors that mediate cellular adhesion to extracellular matrix proteins and serve as bidirectional signal transducers to regulate differentiation, migration, proliferation and cell death. ITGαvβ6 has been shown to be highly expressed on HNSCC as well as lung, colon, breast and pancreas carcinoma and is often associated with poor prognosis (Bandyopadhayay et al., Curr Drug Targets, 2009, 10, 645-52). Thus, it is desirable to generate polypeptide with tumor binding activity targeting the receptor integrin αᵥβ₆ (ITGαᵥβ₆). A linear peptide comprising the integrin binding motives RGD and LXXL is known in the prior art. However, short linear peptides are not particularly stable in a physiological environment and often have low affinities.

The present inventors have overcome these problems by identifying the polypeptides of the present invention, which inter alia provide one or more of the following advantages: (i) good target affinity, (ii) increased stability under physiological conditions, and (iii) improved cancer therapy and diagnosis and evaluation.

### SUMMARY OF THE INVENTION

In a first aspect, the present invention relates to a polypeptide, preferably with the ability of specifically binding to a tumour, which comprises, essentially consists or consists of the amino acid sequence of formula (I) or (II)

(I)
(SEQ ID NO: 1)
R₁-HN-(B₁)ₘ-C₁-(B₂)ₙ-Z₁-Z₂-X₁-R-G-D-L-X₂-X₃-L-Z₃-
(B₃)ₒ-C₂-(B₄)ₚ-COO-R₂
or
(II)
(SEQ ID NO: 2)
R₁-HN-(B₄)ₚ-C₂-(B₃)ₒ-Z₃-L-X₃-X₂-L-D-G-R-X₁-Z₂-Z₁-
(B₂)ₙ-C₁-(B₁)ₘ-COO-R₂

wherein
C₁ and C₂ form a disulfide bond;
X₁ is any amino acid, preferably an amino acid selected from the group consisting of F, W, T, G, A, I, L, M, V, more preferably F and G, most preferably F;
X₂ is any amino acid, preferably an amino acid selected from the group consisting of M, A, I, L, V, G; more preferably M and G, most preferably M;
X₃ is any amino acid, preferably an amino acid selected from the group consisting of Q, D, R and L; more preferably Q and R, most preferably Q;
Z₁ is any amino acid, preferably an amino acid selected from the group consisting of F, M, A, V, I, L, Y and W, more preferably F, Y and W, most preferably F, or not present;
Z₂ is any amino acid, preferably an amino acid selected from the group consisting of T, Q, S, and N, more preferably T and S, most preferably T, or not present;
Z₃ is any amino acid or not present;
B₁ is any amino acid, preferably an amino acid selected from the group consisting of G, A, H, K and R, m is an integer selected from 0, 1, 2 or 3, preferably 2, more preferably B₁ in formula (I) is GH, GK, or GR, and B₁ in formula (II) is HG, KG, or KG, most preferably in formula (I) GR and in formula (II) RG;
B₂ is any amino acid, n is an integer selected from 0, 1, or 2, preferably 0;
B₃ is any amino acid, o is an integer selected from 0, 1, 2 or 3, preferably 0;

B₄ is any amino acid, preferably an amino acid selected from the group consisting of Y, W, P, E, and D, p is an integer selected from 0, 1, 2 or 3, preferably 3, more preferably B₄ in formula (I) is YPD, FPD, WPD, YPE, FPE and WPE and B₄ in formula (II) is DPY, DPF, DPW, EPY, EPF, and EPW, most preferably YPD in formula (I) and DPY in formula (II);
R₁ is H, a therapeutic agent, an imaging agent, a linker group or a linker group linked to a therapeutic agent, or an imaging agent; and
R₂ is H, a therapeutic agent, an imaging agent, a linker group or a linker group linked to a therapeutic agent, or an imaging agent.

In a second aspect, the present invention relates to the polypeptide of the first aspect of the invention for medical use, in particular for use in the treatment of cancer.

In a third aspect, the present invention relates to the polypeptide of the first aspect of the invention for use in the diagnosis and/or evaluation of cancer, preferably for in vivo use.

### FIGURES

FIG. 1 Primary structure of SFTI-1 and SFITGv6: Primary structure and disulfide connectivity of the natural trypsin inhibitor SFTI-1 from sunflower seeds and its exemplary synthetic derivate SFITGv6 extended by the ITGαᵥβ₆ binding motif (Phe-Arg-Gly-Asp-Leu-Met-Gln-Leu, FRGDLMQL) (SEQ ID NO: 11) are shown. The chelating moiety DOTA is N-terminal linked for labeling with metallic radionuclides.

FIG. 2 Western blot analysis of protein lysates derived from HNO97 cells. Using the rabbit monoclonal anti-human epidermal growth factor receptor (EGFR), EGFR was only detected in protein fractions of cell membrane (M), but absent in cytosolic (C) and nuclear fractions (N).

FIG. 3 ITGαᵥβ₆ expression in vitro and in situ: (A) Flow cytometric analysis of ITGαᵥβ₆ expression in cell lines derived from different epithelial carcinomas: head and neck squamous cell carcinoma (HNO97, HNO407, HNO199, HNO210, HNO258), lung carcinoma (LUDLU-1), bladder carcinoma (UM-UC-5), colorectal adenocarcinoma (HT29), ductal carcinoma (T47D), breast carcinoma (MCF7), and one liposarcoma cell line (SW872), which was ITGαᵥβ₆ negative. Bar graphs represent percentage of ITGαᵥβ₆-positive cells of three independent biological replicates. (B−G) Representative immunohistochemical staining against ITGαᵥβ₆ of HNSCC tumors HNO97 (B), HNO407 (C), HNO218a (D), NSCLC-derived brain metastasis NCH1064 (E), breast cancer-derived brain metastasis NCH381d (F), and dysplasia-free mucosa NSH21 (G). Scale bar=50 μm.

FIG. 4 Binding properties of SFPF-10: (A) Unlabeled SFPF-10 (10-10 M to 10-4 M) was used to compete for the binding of ²⁵I-SFPF-10 to HNO97 cells within 60 min. (B) HNO97 cells were exposed to ¹²⁵I- SFPF-10 or ¹⁷⁷Lu-DOTA-SFPF-10 for 10, 30, 60, 120, 240 360, and 420 min. Each value represents mean and standard derivation of three technical replicates.

FIG. 5 In vitro characterization of SFPF-10 and the improved SFITGv6 peptide: (A) Percentage of ¹²⁵I-labeled peptide SFPF-10 (¹²⁵I-SFPF-10) binding to different carcinoma cell lines (HNSCC (HNO97, HNO210, HNO199, HNO258, HNO233), bladder (UM-UC-5), and breast (MCF-7)) after 60 min with (black bars) and without (grey bars) addition of unlabeled peptide (10-6 M) as competitor. (B) HNO97 and UM-UC-5 cells were incubated with ¹²⁵I-SFPF-10 and ¹²⁵I-labeled peptide derivates for 60 min. (C) Binding of SFITGv6 to the cell lines HNO97, HNO199, HNO210, HNO258, UM-UC-5, LUDLU-1, and HT29 was determined after incubation with the ¹²⁵I-labeled SFITGv6 for 60 min with or without addition of the unlabeled peptide (10-6 M) as competitor. (D) HNO97 cells were exposed to ¹²⁵I-SFITGv6 and ¹⁷⁷Lu-DOTA-SFITGv6 for 10, 30, 60, 120, 360, and 420 min, respectively. (E) Total bound peptide and internalization of ¹⁷⁷Lu-DOTA-SFITGv6 in HNO97 cells was determined after incubation for 10, 30, 60, 120, and 240 min, respectively. (F) HNO97 cells were exposed to ¹⁷⁷Lu-DOTA-SFITGv6 for one hour, the medium was replaced by non-radioactive medium and the radioactivity in cell lysates was determined after 0, 1, 2, and 4 hours. The radioactivity was calculated as percent applied dose/106 cells. Each value represents mean and standard derivation of three technical replicates.

FIG. 6 SFITGv6 is characterized by high stability and affinity for ITGαᵥβ₆: (A) SFITGv6, A2OFMDV2, TP H2009.1 and HBP-1, respectively, (10-10 M to 10-4 M) were used to compete for the binding of ¹²⁵I-SFITGv6 to HNO97 cells within 60 min and the radioactivity was calculated as % applied dose/106 cells. Each value represents mean and standard derivation of three technical replicates. The affinity of (B) ITGαᵥβ₆ (20, 30, 40 and 50 μg/mL) and (C) ITGαᵥβ₃ (1, 2, 4 and 7 μg/mL) to immobilized SFITGv6 (loading level: 18 RU) was measured with a flow rate of 30 μL/mL by SPR analysis. The SPR-sensograms (n=3) were evaluated with the BiaCore evaluation software (black curves) and correspond to the experimentally fitted red curves. KD values were determined by a 1:1 langmuir model fit of the SPR-sensograms. (D) The stability of ¹²⁵I-SFITGv6 in human serum was evaluated by radio-HPLC analysis after 15 min, 1, 2, and 24 hours, respectively.

FIG. 7 Evaluation of ¹²⁵I-SFITGv6 binding to breast- and colon carcinoma cell lines and the internalization and efflux of ¹²⁵I-SFITGv6 from HNO97 cells. (A) Binding of ¹²⁵I-SFITGv6 after 60 min to carcinoma cell lines MCF-7, and T47D as well as to the liposarcoma cell line SW872. (B) Internalization of ¹²⁵I-SFITGv6 in HNO97 cells was determined after incubation for 10, 30, 60, 120 and 240 min. (C) HNO97 cells were exposed to ¹²⁵I-SFITGv6 for 1 hour, the medium was replaced by non-radioactive medium and the radioactivity (% applied dose/106 cells) in cell lysates was determined after 0, 60, 120, and 240 min. Each value represents mean and standard derivation of three technical replicates.

FIG. 8 Small-animal PET imaging and biodistribution of SFITGv6: (A) Small-animal PET imaging was performed with HNO97 xenografted Balb/c nu/nu mice 130 min after injection of 50 MBq (2 nmol) ⁶⁸Ga-DOTA-labeled SFITGv6 and (B) mice pretreated by intraperitoneal administration of 100 μL SFITGv6 (1 mM/H20) as competitor 30 min prior to the injection of the radiolabeled peptide. (C) Based on the SUV values a time activity curve was calculated. (D) The accumulation of ¹⁷⁷Lu-DOTA-SFITGv6 in tumors and organs of HNO97 xenografted Balb/c nu/nu mice was measured 30, 60, 120, 240, and 480 min after injection and calculated as % ID/g. Data are shown as mean of three per time point±SD.

FIG. 9 SFITGv6 peptide binds specifically on epithelial tumor cells in situ: Patient-derived HNO97 mouse xenograft (A) and HNO444 (D) kryosections were performed with the biotinylated PEG(12)-SFITGv6 peptide (10-5 M) in antibody diluents and detected by the Vectastain Elite ABC Kit. Peptide specificity was ensured by (B, E) a scrambled (GRD) PEG(12)-SFITGv6 derivate, and (C, F) DOTATOC (DOTA(0)-Phe(1)-Tyr(3))octreotid). Scale bar=50 μm.

FIG. 10 SFITGv6 specifically accumulates in tumor lesions of patients: PET/CT scans were performed in two tumor patients suffering from (A, B) recurrent hypopharynx tumor and (C, D) non-small cell lung cancer and after application of (A, C) ¹⁸F-FDG and (B, D) ⁶⁸Ga-DOTA-SFITGv6. The upper row shows maximal intensity projections (MIP) of both scans in these patients. Below are transaxial slices of the PET/CT fusion images. Red arrows indicate lesions seen in the PET/CT scans.

FIG. 11 Histochemical peptide staining of brain metastasis: Histochemical staining of representative sections of (A, C) breast cancer-derived brain metastasis (NCH640k), and (D−F) NSCLC-derived brain metastasis (NCH2407A) was performed with (A, D) biotinylated PEG(12)-SFITGv6, (B, E) scrambled (GRD) PEG(12)-SFITGv6 derivate, and (C, F) DOTATOC. Scale bar=50 μm.

FIG. 12 Histochemical peptide staining of carcinoma-free lymph nodes. Representative staining of the carcinoma-free lymph node (HNO299) with (A) biotinylated SFITGv6 peptide, (B) scrambled (GRD) PEG(12)-SFITGv6 derivate, and (C) DOTATOC. Scale bar=50 μm.

FIG. 13 Flow cytometry analysis of ITGαvβ6 expression in the HNSCC cell lines HNO97, HNO399, HNO199, HNO210, HNO223. Bar graphs represent percentage of ITGαvβ6-positive cells of three independent biological replicates.

FIG. 14 (A) Binding capacities of ¹²⁵I-labeled peptides to different HNSCC cell lines. Bars represent the percentage of total bound ¹²⁵I-labeled peptide (SFLAP3, SFITGv6, SFLAP1, SFTNC, SFFN1, and SFVTN) after 60 min per applied dose per 1×10⁶ cells of different HNSCC cell lines (HNO97, HNO399, HNO223, HNO210, and HNO199). By increasing the amount of unlabeled peptide which functioned as a competitor the binding of ¹²⁵I-labeled SFLAP (B) and SFITGv6 (C) to the HNSCC cell lines HNO97, HNO399, and HNO223 could be successively decreased (calculated as percentage of applied dose/10⁶ cells). At a competitor concentration of 10⁻⁶ M the competition was almost completed. (D) Surface plasmon resonance (SPR) binding analysis of study the molecular interaction of the peptide SFLAP3 and the heterodimeric receptor ITGαᵥβ₆ (1, 2, 3 and 4 μg/mL). The final concentration of bound ligand, expressed in response units (RU), is calculated by subtracting the reference RU from the ligand RU.). Error bars show the standard error of the mean of technical triplicates.

FIG. 15 (A) SPR analysis (n=3) of ITGαVβ3 (applied concentrations: 15, 30, 35 and 40 μg/mL) to immobilized SFLAP3 (loading level: 18 RU) measured with a flow rate of 30 mL/mL. (B) The stability of 177Lu-DOTA-SFLAP3 in human serum was evaluated by radio-HPLC analysis after 10, 60, 120, 240 min and 24 hrs.

FIG. 16 The amount of bound peptide (percentage of bound peptide per applied dose per 1×10⁶ HNSCC cells) increased over time. Binding of both, ¹⁷⁷Lu-DOTA-labeled SFLAP3 (A) and SFITGv6 (B) increased from 10 min to 480 min in all analyzed cell lines (HNO97, HNO399, and HNO223). Error bars show the standard error of the mean (SEM) of technical triplicates.

FIG. 17 Amount of total bound and internalized peptide by HNO97 cells was determined after incubation for 10, 30, 60, 120, and 240 min, respectively. Total bound (grey bars) versus internalized (black bars) ¹⁷⁷Lu-DOTA-labeled SFILAP3 (A) and SFITGv6 (B). Time-dependent efflux of the ¹⁷⁷Lu-labeled peptides SFLAP3 (C) and SFITGv6 (D). HNSCC cell cultures HNO97, HNO399, and HNO223 (1×10⁶ each) were exposed to ¹⁷⁷Lu-labeled SFLAP3 (C) and SFITGv6 (D) for 60 min. Medium was replaced by non-radioactive culture medium and measurements were performed at time point 0, 10, 30, 60, 120, 240 min. Error bars represent standard error of the mean of technical triplicates.

FIG. 18 Total bound (bound+internalized) and internalized 177Lu-DOTA-labeled SFLAP3 (A, C) SFLAP3 and (B, D) SFITGv6 on/in 1×106 HNO399 (A, B) and HNO223 tumor cells (C, D). Each value represents mean and standard derivation of three technical replicates.

FIG. 19 Small animal PET imaging of HNO97—(A) and HNO399-derived (C) tumor xenografts, both 60 min after injection of ⁶⁸Ga-DOTA-SFLAP3. (B+D) Biodistribution of ⁶⁸Ga-DOTA-SFLAP3 (% ID/g) in several organs measured 30, 60, 120, 240, and 360 min after injection of ¹⁷⁷Lu-DOTA-SFLAP3 to HNO97 (B) and HNO399 (D) tumor bearing mice. Error bars represent standard error of the mean of biological triplicates (n=3 mice).

FIG. 20 (D) Small-animal PET imaging of HNO97 xenograft mouse 140 min after injection of 68Ga-DOTA-SFLAP3 (37.9 MBq) and (A) time activity curve (SUVmean) up to 60 min. (C) Time activity curve (SUVmean) up to 60 min and small-animal PET imaging of HNO97 xenograft mouse (B) 60 min and (E) 140 min after injection of 68Ga-DOTA-SFITGv6 (30 MBq). (F) Biodistribution of 177Lu-DOTA-SFITGv6 (% ID/g) in tumors and organs of HNO97 xenograft mice (n=3) measured 30, 60, 120, 240, and 360 min after injection of 1 nmol (1 MBq) peptide. Data are shown as mean of three per time point±SD.

FIG. 21 (B) Time activity curve (SUVmean) calculated for the 68Ga-DOTA-SFLAP3 small-animal PET imaging of HNO399 xenograft mouse. (A) Small-animal PET imaging and (C) time activity curve (SUVmean) of a HNO399 xenograft mouse 60 min after injection of 68Ga-DOTA-SFITGv6 (34 MBq).

FIG. 22 (A, B) Small-animal PET imaging and (C, D) time activity curves (SUVmean) of HNO223 xenograft mice 60 min after injection of (A, C) 68Ga-DOTA-SFLAP3 (27 MBq) and (B, D) 68Ga-DOTA-SFITGv6 (34 MBq). (E) Biodistribution of radioactivity (% ID/g) in tumors and organs of HNO223 xenograft mice (n=3) measured 30, 60, 120, 240, and 360 min after injection of 1 nmol (1 MBq) 177Lu-DOTA-SFLAP3. Data are shown as mean of three per time point±SD.

FIG. 23 Histochemical peptide staining of HNSCC cyrosections of patient-derived HNO97 xenografted tumors, HNO399 and HNO210 tumors sections performed with 10⁻⁵ M biotinylated PEG(12)-labeled SFLAP3 and SFITGv6. As a control the scrambled SFLAP3 derivate GRD was applied. Scale bar=50 μm.

FIG. 24 Positron Emission Tomography/Computed Tomography (PET/CT) scans of a HNSCC tumor patient after application of 322 MBq ⁶⁸Ga-DOTA-SFLAP3. (A−D) transaxial slices of the PET/CT fusion images. Yellow arrows indicate peptide accumulation.

FIG. 25 Binding properties of SFLAP3 and its variants on different pancreatic cancer cell lines. A) Amino acid sequence of SFLAP3 and 3-D structure of the sunflower-trypsin inhibitor peptide using PyMol (PDB:1jbl) grey=scaffold, blue=variable loop, yellow=disulfide. B) Cellular uptake of ¹²⁵I-SFLAP3 to pancreatic cancer cell lines after 60 min (ASPC-1, BXPC-3, Capan-1, Capan-2 and MiaPaCa-2). C) SFLAP3 variants based on the native surrounding amino acids of the latency-associated peptide 3 (LAP3) binding sequence. D) Cellular uptake of the ¹²⁵I-SFLAP3 variants. The radioactivity was stated as percent of applied dose/106 cells. Mean values and standard deviation (n=3).

FIG. 26 In vitro characterization of DOTA-SFLAP3, DOTA-SFLAP3K and DOTA-SFLAP3-trimer on Capan-2 cells. A) Uptake of ¹⁷⁷Lu-DOTA-SFLAP3, ¹⁷⁷Lu-DOTA-SFLAP3K and ¹⁷⁷Lu-DOTA-SFLAP3-trimer on Capan-2 cells for 10 and 60 min. B) Competition assay of ¹²⁵I-DOTA-SFLAP3, ¹²⁵I-DOTA-SFLAP3K and ¹²⁵I-DOTA-SFLAP3-trimer with defined amounts of unlabelled peptide (10⁻⁶ M to 10⁻¹⁰ M or 10⁻⁷ M to 10⁻¹¹) for 60 min on Capan-2 cells. C) Internalization rate of ¹⁷⁷Lu-DOTA-SFLAP3, ¹⁷⁷Lu-DOTA-SFLAP3K and ¹⁷⁷Lu-DOTA-SFLAP3-Trimer was determined after 10, 60, 120 and 240 min at 37° C. on Capan-2 cells. D) Efflux assay was performed after a one hour incubation of ¹⁷⁷Lu-DOTA-SFLAP3, ¹⁷⁷Lu-DOTA-SFLAP3K and ¹⁷⁷Lu-DOTA-SFLAP3-trimer. The radioactive medium was replaced and after an incubation of 60, 120, 240 or min the cell bound activity measured. The radioactivity was stated as percent of applied dose/10⁶ cells. Mean values and standard deviation (n=3).

FIG. 27 In vivo imaging and biodistriobution. A) PET imaging of Capan-2 xenograft balb/c nu/nu mouse at different time points (0 to 20 min, 20 to 40 min, 40 to 60 min and 120 to 140 min) after injection of 25 MBq ⁶⁸Ga-DOTA-SFLAP3. B/C) Small animal imaging of Capan-2 bearing balb/c nu/nu mice 120 to 140 min after injection of 25 MBq ⁶⁸Ga-DOTA-SFLAP3K (B) or 25 MBq ⁶⁸Ga-DOTA-SFLAP3-Trimer (C). D) Biodistribution of 1 MBq ¹⁷⁷Lu-DOTA-SFLAP3 in Capan-2 xenografted Balb/c nu/nu mice 0.5, 1, 2, 4, 6 and 24 h after injection. Mean values and standard deviation (n=3) are expressed as % ID/g.

FIG. 28 Diagnostic and therapy imaging of a late stage pancreatic cancer patient. A) PET/CT scans were performed with a pancreatic cancer patient. Images were taken 1 and 3 hours after injection of ⁶⁸Ga-DOTA-SFLAP3. B) Scintigraphy one day after injection of 6.5 GBq ¹⁷⁷Lu-DOTA-SFLAP3 (R=right, L=left, V=ventral, D=dorsal).

FIG. 29 Diagnostic and therapy in ovarian cancer patients. A/B) PET/CT scans of two ovarian cancer patients. Images were taken 1 and 3 hours after injection of ⁶⁸Ga-DOTA-SFLAP3. C/D) Scintigraphy (of patient 5A/5B) one day after administration of 6 GBq and 7.4 GBq ¹⁷⁷Lu-DOTA-SFLAP3. Arrows indicate clearly visible metastasis (R=right, L=left, V=ventral, D=dorsal).

### DETAILED DESCRIPTION OF THE INVENTION

Employing a phage display library based on the molecular scaffold of SFTI-1 for alternate biopanning on HNO97 cells or selected membrane protein fractions of this cell line the present inventors identified a peptide containing the RGDKXXL motif (SEQ ID NO: 42). The amino acid substitution of K⁴ to L⁴ in the original peptide improved the binding and affinity of the RGDLXXL (SEQ ID NO: 43) containing SFTI-1 derivate (SFITGv6) for a variety of HNSCC and other tumor cell lines of epithelial origin. SFITGv6 demonstrated an outstanding stability in human serum over a period of 24 hours and high affinity (K_{D}=14.8 nM) for ITGαᵥβ_{b}. In correlation to the ITGαvβ6 expression of different cell lines as measured by FACS analysis the ¹²⁵I-labeled peptide displayed specific binding to HNO97 cells and other HNSCC cells as well as to further carcinoma cell lines of different origin including lung, bladder and colon with an average of 7% which was competed to more than 90% by addition of 10⁻⁶ M unlabeled analog. The experiments concerning the binding kinetics and internalization revealed a maximal binding of ¹²⁵I-SFITGv6 to HNO97 cells after exposure for 60 min followed by a decrease to less than 15%. On the contrary, an increasing uptake and internalization of ¹⁷⁷Lu-DOTA-SFITGv6 to values of up to 57.3% and up to 37.4%, respectively, as well as retention of more than 50% of the applied dose for at least 4 hours in HNO97 cells was measured. This indicates a time-dependent intracellular deionization of ¹²⁵I-labeled peptide followed by the efflux of free radioiodine. The long-lasting accumulation of the ¹¹⁷Lu-DOTA labeled peptide in tumor cells, however, allows for late imaging and possibly for a therapeutic application.

Using ⁶⁸Ga-DOTA-SFITGv6, the inventors were able to successfully and selectively image the ITGαᵥβ₆-expressing HNO97 tumor of Balb/c mice within 20 min in a small animal PET. The rapid clearance of unbound and unspecifically bound activity from the blood and the surrounding tissues resulted in an excellent tumor-to-background ratio 40 min after the injection remaining for at least 140 min. In parallel to the in vitro competition experiments a complete inhibition of ⁶⁸Ga-DOTA-SFITGv6 accumulation in the tumor was achieved by injecting a nonradioactive analog before. This is evidence for the selective in vivo imaging of a tumor that endogenously expresses ITGαᵥβ₆. The biodistribution data revealed a significantly higher accumulation of ¹⁷⁷Lu-DOTA-SFITGv6 in the HNO97 tumor as compared to healthy tissues even after 4 and 6 hours (Table 1). However, in contrast to the binding kinetics in culture a washout of the radioactivity from the HNO97 tumor with time was noticed which might be due to a fast clearance of the tracer from the blood within the first 30 min. Of particular interest is the high and constant level of radioactivity of almost 42% ID/g in the kidneys in HNO97 tumor bearing Balb/c nude mice with hardly any clearance.

Peptide-based histochemical staining of HNO97-xenotransplanted mouse tumors and different human HNSCC tumor tissues revealed a specific, strong and homogeneous binding of biotinylated SFITGv6. As expected from the binding to different squamous and adenocarcinoma cell lines in vitro, the peptide displayed a moderate to strong binding in brain metastases derived from breast cancer and NSCLC, respectively. Since tumor-free lymph nodes did not show any staining inflammation-associated binding of the peptide can be excluded. Accordingly, tumor-specific but not inflammation-associated binding of SFITGv6 was observed in PET/CT performed in two patients suffering from either recurrent hypopharynx carcinoma or NSCLC after application of ⁶⁸Ga-DOTA-SFITGv6 and ¹⁸F-FDG, respectively. In line with the small animal PET images of HNO97 xenografts the SUV values of SFITGv6 in these patients persisted from 1 to 3 hours after injection. Interestingly in contrast to ⁶⁸Ga-DOTA-SFITGv6 ¹⁸F-FDG accumulation was detected in inflammatory lesions and reactive lymph nodes of both patients. The property of FDG accumulation in inflammatory or infectious processes is well-known and may occasionally lead to false positive evaluation and incorrect up-staging of tumor patients with a tremendous impact on therapy management. Thus, in ITGαᵥβ₆-positive tumors the peptide-based tracer provided a clear advantage over FDG with respect to false positive lesions.

SFITGv6 also accumulated in the kidneys, the bowel, the stomach and in the thyroid of both patients. Interestingly, the localization of the radioactivity in the bowel changed with time indicating the excretion of the tracer. In fact, regions of interest drawn in jejunum, terminal ileum and cecum revealed a decrease of the activity in the jejunum and a time-dependent increase in terminal ileum and cecum. This is evidence for a secretion of SFITGv6 into the stomach or duodenum/jejunum followed by an intraluminal transport to the terminal ileum and the cecum. With regard to the identification of small tumor lesions in the abdomen or a peritonitis carcinomatosa the administration of laxatives might be necessary to reduce intra-bowel activity and avoid a high background.

In conclusion, the polypeptide of the present invention is a novel stable ITGαᵥβ₆-specific polypeptide with high affinity for a variety of HNSCC and other tumors. Due to the accumulation of the peptide in different tumors but not in inflammatory lesions and normal tissues of tumor patients the peptide of the present invention represents a promising tracer for imaging and therapy of cancer, particularly of ITGαᵥβ₆-positive carcinoma.

Accordingly, in a first aspect the present invention relates to a polypeptide, preferably with the ability of specifically binding to a tumour, which comprises, essentially consists or consists of the amino acid sequence of formula (I) or (II)

(I)
R₁-HN-(B₁)ₘ-C₁-(B₂)ₙ-Z₁-Z₂-X₁-R-G-D-L-X₂-X₃-L-Z₃-
(B₃)ₒ-C₂-(B₄)ₚ-COO-R₂
or
(II)
R₁-HN-(B₄)ₚ-C₂-(B₃)ₒ-Z₃-L-X₃-X₂-L-D-G-R-X₁-Z₂-Z₁-
(B₂)ₙ-C₁-(B₁)ₘ-COO-R₂

wherein
C₁ and C₂ form a disulfide bond;
X₁ is any amino acid, preferably an amino acid selected from the group consisting of F, W, T, G, A, I, L, M, V, more preferably F and G, most preferably F;
X₂ is any amino acid, preferably an amino acid selected from the group consisting of M, A, I, L, V, G; more preferably M and G, most preferably M;
X₃ is any amino acid, preferably an amino acid selected from the group consisting of Q, D, R and L; more preferably Q and R, most preferably Q;
Z₁ is any amino acid, preferably an amino acid selected from the group consisting of F, M, A, V, I, L, Y and W, more preferably F, Y and W, most preferably F, or not present;
Z₂ is any amino acid, preferably an amino acid selected from the group consisting of T, Q, S, and N, more preferably T and S, most preferably T, or not present;
Z₃ is any amino acid or not present;
B₁ is any amino acid, preferably an amino acid selected from the group consisting of G, A, H, K and R, m is an integer selected from 0, 1, 2 or 3, preferably 2, more preferably B₁ in formula (I) is GH, GK, or GR, and B₁ in formula (II) is HG, KG, or KG, most preferably in formula (I) GR and in formula (II) RG;
B₂ is any amino acid, n is an integer selected from 0, 1, or 2, preferably 0;
B₃ is any amino acid, o is an integer selected from 0, 1, 2 or 3, preferably 0;
B₄ is any amino acid, preferably an amino acid selected from the group consisting of Y, W, P, E, and D, p is an integer selected from 0, 1, 2 or 3, preferably 3, more preferably B₄ in formula (I) is YPD, FPD, WPD, YPE, FPE and WPE and B₄ in formula (II) is DPY, DPF, DPW, EPY, EPF, and EPW, most preferably YPD in formula (I) and DPY in formula (II);
R₁ is H, a therapeutic agent, an imaging agent, a linker group or a linker group linked to a therapeutic agent, or an imaging agent; and
R₂ is H, a therapeutic agent, an imaging agent, a linker group or a linker group linked to a therapeutic agent, or an imaging agent.

Preferably, the present invention relates to a polypeptide comprising, essentially consisting or consisting of the amino acid sequence of formula (III) or (IV)

(III)
R₁-HN-(B₁)ₘ-C₁-Z₁-X₁-R-G-D-L-X₂-X₃-L-C₂-(B₄)ₚ-COO-R₂
or
(IV)
R₁-HN-(B₁)ₘ-C₂-L-X₃-X₂-L-D-G-R-X₁-Z₁-C₁-(B₄)ₚ-COO-R₂

wherein
C₁ and C₂ form a disulfide bond;
X₁ is any amino acid, preferably an amino acid selected from the group consisting of F and G, most preferably F;
X₂ is any amino acid, preferably an amino acid selected from the group consisting of M and G, most preferably M;
X₃ is any amino acid, preferably an amino acid selected from the group consisting of Q and R, most preferably Q;
Z₁ is any amino acid, preferably T and S, most preferably T;
B₁ is any amino acid, preferably an amino acid selected from the group consisting of G, A, H, K and R, m is an integer selected from 0, 1, 2 or 3, preferably 2, more preferably B₁ in formula (III) is

GH, GK, or GR, and B₁ in formula (II) is HG, KG, or KG, most preferably in formula (I) GR and in formula (IV) RG;
B₄ is any amino acid, preferably an amino acid selected from the group consisting of Y, W, P, E, and D, p is an integer selected from 0, 1, 2 or 3, preferably 3, more preferably B₄ in formula (III) is YPD, FPD, WPD, YPE, FPE and WPE and B₄ in formula (IV) is DPY, DPF, DPW, EPY, EPF, and EPW, most preferably YPD in formula (III) and DPY in formula (IV);
R₁ is H, a therapeutic agent, an imaging agent, a linker group or a linker group linked to a therapeutic agent, or an imaging agent; and
R₂ is H, a therapeutic agent, an imaging agent, a linker group or a linker group linked to a therapeutic agent, or an imaging agent.

It is further preferred that the polypeptide of the present invention comprises, essentially consists or consists of the amino acid sequence of formula (V) or (VI)

(V)
(SEQ ID NO: 3)
R₁-NH-G-R-C₁-T-F-R-G-D-L-M-Q-L-C₂-Y-P-D-COO-R₂
or
(VI)
(SEQ ID NO: 4)
R₁-HN-D-P-Y-C₂-L-Q-M-L-D-G-R-F-T-C₁-G-R-COO-R₂

wherein
C₁ and C₂ form a disulfide bond;
R₁ is H, a therapeutic agent, an imaging agent, a linker group or a linker group linked to a therapeutic agent, or an imaging agent; and
R₂ is H, a therapeutic agent, an imaging agent, a linker group or a linker group linked to a therapeutic agent, or an imaging agent.

It is further preferred that the polypeptide of the present invention comprises, essentially consists or consists of the amino acid sequence of formula (V) or (VI)

(VII)
(SEQ ID NO: 5)
R₁-NH-G-R-C₁-T-G-R-G-D-L-G-R-L-C₁-Y-P-D-COO-R₂
or
(VIII)
(SEQ ID NO: 6)
R₁-HN-D-P-Y-C₂-L-R-G-L-D-G-R-G-T-C₁-G-R-COO-R₂

wherein
C₁ and C₂ form a disulfide bond;
R₁ is H, a therapeutic agent, an imaging agent, a linker group or a linker group linked to a therapeutic agent, or an imaging agent; and
R₂ is H, a therapeutic agent, an imaging agent, a linker group or a linker group linked to a therapeutic agent, or an imaging agent.

It is further preferred that the polypeptide of the present invention has the amino acid sequence

(SEQ ID NO: 7)
NH₂-G-R-C₁-T-F-R-G-D-L-M-Q-L-C₁-Y-P-D-COOH
or
(SEQ ID NO: 8)
NH₂-D-P-Y-C₂-L-Q-M-L-D-G-R-F-T-C₁-G-R-COOH

wherein
C₁ and C₂ form a disulfide bond.

Alternatively, the polypeptide of the present invention has preferably the sequence

(SEQ ID NO: 9)
NH₂-G-R-C₁-T-G-R-G-D-L-G-R-L-C₁-Y-P-D-COOH
or
(SEQ ID NO: 10)
NH₂-D-P-Y-C₂-L-R-G-L-D-G-R-G-T-C₁-G-R-COOH

wherein
C₁ and C₂ form a disulfide bond.

Preferably, the present invention relates to a polypeptide comprising, essentially consisting or consisting of the amino acid sequence of formula (IX) or (X)

(IX)
(SEQ ID NO: 22)
R₁-HN-(B₁)ₘ-C₁-(B₂)ₙ-Z₁-Z₂-X₁-R-G-D-L-X₂-X₃-L-Z₃-
(B₃)ₒ-C₂-(B₄)ₚ-COO-R₂
or
(X)
(SEQ ID NO: 23)
R₁-HN-(B₄)ₚ-C₂-(B₃)ₒ-Z₃-L-X₃-X₂-L-D-G-R-X₁-Z₂-Z₁-
(B₂)ₙ-C₁-(B₁)ₘ-COO-R₂

wherein
C₁ and C₂ form a disulfide bond; X₁ is any amino acid, preferably an amino acid selected from the group consisting of F, W, T, G, A, I, L, M, V, more preferably F and G, most preferably G, or not present;
X₂ is any amino acid, preferably an amino acid selected from the group consisting of M, A, I, L, V, G; more preferably M and G, most preferably G;
X₃ is any amino acid, preferably an amino acid selected from the group consisting of Q, D, R and L; more preferably Q and R, most preferably R;
Z₁ is any amino acid, preferably an amino acid selected from the group consisting of T, Q, S, and N, more preferably T and S, most preferably T, or not present;
Z₂ is any amino acid, preferably an amino acid selected from the group consisting of F, M, A, V, I, L, H, Y and W, more preferably F, H, Y and W, most preferably H, or not present;
Z₃ is any amino acid or not present;
B₁ is any amino acid, preferably an amino acid selected from the group consisting of G, A, H, K and

R, m is an integer selected from 0, 1, 2 or 3, preferably 2, more preferably B₁ in formula (IX) is GH, GK, or GR, and B₁ in formula (X) is HG, KG, or RG, most preferably in formula (IX) GR and in formula (X) RG;
B₂ is any amino acid, n is an integer selected from 0, 1, or 2, preferably 0;
B₃ is any amino acid, preferably an amino acid selected from the group consisting of G, A, H, K and R, o is an integer selected from 0, 1, 2 or 3, preferably 1 or 2, more preferably B₃ in formula (IX) is K or KK, and B₃ in formula (X) is K or KK, most preferably in formula (IX) K and in formula (X) K;
B₄ is any amino acid, preferably an amino acid selected from the group consisting of Y, W, P, E, and D, p is an integer selected from 0, 1, 2 or 3, preferably 3, more preferably B₄ in formula (IX) is YPD, FPD, WPD, YPE, FPE and WPE and B₄ in formula (X) is DPY, DPF, DPW, EPY, EPF, and EPW, most preferably YPD in formula (IX) and DPY in formula (X);
R₁ is H, a therapeutic agent, an imaging agent, a linker group or a linker group linked to a therapeutic agent, or an imaging agent; and
R₂ is H, a therapeutic agent, an imaging agent, a linker group or a linker group linked to a therapeutic agent, or an imaging agent.

Preferably, the present invention further relates to a polypeptide comprising, essentially consisting or consisting of the amino acid sequence of formula (XI) or (XII)

(XI)
(SEQ ID NO: 24)
R₁-HN-(B₁)ₘ-C₁-Z₁-Z₂-X₁-R-G-D-L-X₂-X₃-L-Z₃-(B₃)ₒ-
C₂-(B₄)ₚ-COO-R₂
or
(XII)
(SEQ ID NO: 25)
R₁-HN-(B₄)ₚ-C₂-(B₃)ₒ-Z₃-L-X₃-X₂-L-D-G-R-X₁-Z₂-Z₁-
C₁-(B₁)ₘ-COO-R₂

wherein
C₁ and C₂ form a disulfide bond;
X₁ is any amino acid, preferably an amino acid selected from the group consisting F and G, most preferably G, or not present;
X₂ is any amino acid, preferably an amino acid selected from the group consisting of M and G, most preferably G;
X₃ is any amino acid, preferably an amino acid selected from the group consisting of Q and R, most preferably R;
Z₁ is any amino acid, preferably an amino acid selected from the group consisting of T and S, most preferably T, or not present;
Z₂ is any amino acid, preferably an amino acid selected from the group consisting of F, H, Y and W, most preferably H, or not present;
Z₃ is any amino acid or not present;
B₁ is any amino acid, preferably an amino acid selected from the group consisting of G, A, H, K and R, m is an integer selected from 0, 1, 2 or 3, preferably 2, more preferably B₁ in formula (XI) is GH, GK, or GR, and B₁ in formula (XII) is HG, KG, or RG, most preferably in formula (XI) GR and in formula (XII) RG;
B₃ is any amino acid, preferably an amino acid selected from the group consisting of G, A, H, K and R, o is an integer selected from 0, 1, 2 or 3, preferably 1 or 2, more preferably B₃ in formula (XI) is K or KK, and B₃ in formula (XII) is K or KK, most preferably in formula (XI) K and in formula (XII) K;
B₄ is any amino acid, preferably an amino acid selected from the group consisting of Y, W, P, E, and D, p is an integer selected from 0, 1, 2 or 3, preferably 3, more preferably B₄ in formula (XI) is YPD, FPD, WPD, YPE, FPE and WPE and B₄ in formula (XII) is DPY, DPF, DPW, EPY, EPF, and EPW, most preferably YPD in formula (XI) and DPY in formula (XII);
R₁ is H, a therapeutic agent, an imaging agent, a linker group or a linker group linked to a therapeutic agent, or an imaging agent; and
R₂ is H, a therapeutic agent, an imaging agent, a linker group or a linker group linked to a therapeutic agent, or an imaging agent.

Preferably, the present invention further relates to a polypeptide comprising, essentially consisting or consisting of the amino acid sequence of formula (XIII) or (XIV)

(XIII)
(SEQ ID NO: 26)
R₁-HN-G-R-C₁-T-Z₂-X₁-R-G-D-L-G-R-L-(B₃)ₒ-C₂-Y-
P-D-COO-R₂
or
(XIV)
(SEQ ID NO: 27)
R₁-HN-D-P-Y-C₂-(B₃)ₒ-L-R-G-L-D-G-R-X₁-Z₂-T-C₁-
R-G-COO-R₂

wherein
C₁ and C₂ form a disulfide bond;
X₁ is any amino acid, preferably an amino acid selected from the group consisting F and G, most preferably G, or not present;
Z₂ is any amino acid, preferably an amino acid selected from the group consisting of F, H, Y and W, most preferably H, or not present;
B₃ is any amino acid, preferably an amino acid selected from the group consisting of G, A, H, K and R, o is an integer selected from 0, 1, 2 or 3, preferably 1 or 2, more preferably B₃ in formula (XIII) is K or KK, and B₃ in formula (XIV) is K or KK, most preferably in formula (XIII) K and in formula (XIV) K;
R₁ is H, a therapeutic agent, an imaging agent, a linker group or a linker group linked to a therapeutic agent, or an imaging agent; and
R₂ is H, a therapeutic agent, an imaging agent, a linker group or a linker group linked to a therapeutic agent, or an imaging agent.

Preferably, the present invention further relates to a polypeptide comprising, essentially consisting or consisting of the amino acid sequence of formulas (XV) to (XXVI):

(XV)
(SEQ ID NO: 28)
R₁-HN-G-R-C₁-T-G-R-G-D-L-G-R-L-C₂-Y-P-D-COO-R₂
or
(XVI)
(SEQ ID NO: 29)
R₁-HN-D-P-Y-C₂-L-R-G-L-D-G-R-G-T-C₁-R-G-COO-R₂
or
(XVII)
(SEQ ID NO: 30)
R₁-HN-G-R-C₁-T-H-G-R-G-D-L-G-R-L-K-C₂-Y-P-D-COO-R₂
or
(XVIII)
(SEQ ID NO: 31)
R₁-HN-D-P-Y-C₂-K-L-R-G-L-D-G-R-G-H-T-C₁-R-G-COO-R₂
or
(XIX)
(SEQ ID NO: 32)
R₁-HN-G-R-C₁-T-H-G-R-G-D-L-G-R-L-C₂-Y-P-D-COO-R₂
or
(XX)
(SEQ ID NO: 33)
R₁-HN-D-P-Y-C₂-L-R-G-L-D-G-R-G-H-T-C₁-R-G-COO-R₂
or
(XXI)
(SEQ ID NO: 34)
R₁-HN-G-R-C₁-T-G-R-G-D-L-G-R-L-K-C₂-Y-P-D-COO-R₂
or
(XXII)
(SEQ ID NO: 35)
R₁-HN-D-P-Y-C₂-K-L-R-G-L-D-G-R-G-T-C₁-R-G-COO-R₂
or
(XXIII)
(SEQ ID NO: 36)
R₁-HN-G-R-C₁-T-R-G-D-L-G-R-L-K-K-C₂-Y-P-D-COO-R₂
or
(XXIV)
(SEQ ID NO: 37)
R₁-HN-D-P-Y-C₂-K-K-L-R-G-L-D-G-R-T-C₁-R-G-COO-R₂
or
(XXV)
(SEQ ID NO: 38)
R₁-HN-G-R-C₁-T-R-G-D-L-G-R-L-K-C₂-Y-P-D-COO-R₂
or
(XXVI)
(SEQ ID NO: 39)
R₁-HN-D-P-Y-C₂-K-L-R-G-L-D-G-R-T-C₁-R-G-COO-R₂

wherein
C₁ and C₂ form a disulfide bond;
R₁ is H, a therapeutic agent, an imaging agent, a linker group or a linker group linked to a therapeutic agent, or an imaging agent; and
R₂ is H, a therapeutic agent, an imaging agent, a linker group or a linker group linked to a therapeutic agent, or an imaging agent.

Alternatively, the polypeptide of the present invention has preferably the sequence

(SEQ ID NO: 28)
H₂N-G-R-C₁-T-G-R-G-D-L-G-R-L-C₂-Y-P-D-COOH
or
(SEQ ID NO: 29)
H₂N-D-P-Y-C₂-L-R-G-L-D-G-R-G-T-C₁-R-G-COOH
or
(SEQ ID NO: 30)
H₂N-G-R-C₁-T-H-G-R-G-D-L-G-R-L-K-C₂-Y-P-D-COOH
or
(SEQ ID NO: 31)
H₂N-D-P-Y-C₂-K-L-R-G-L-D-G-R-G-H-T-C₁-R-G-COOH
or
(SEQ ID NO: 32)
H₂N-G-R-C₁-T-H-G-R-G-D-L-G-R-L-C₂-Y-P-D-COOH
or
(SEQ ID NO: 33)
H₂N-D-P-Y-C₂-L-R-G-L-D-G-R-G-H-T-C₁-R-G-COOH
or
(SEQ ID NO: 34)
H₂N-G-R-C₁-T-G-R-G-D-L-G-R-L-K-C₂-Y-P-D-COOH
or
(SEQ ID NO: 35)
H₂N-D-P-Y-C₂-K-L-R-G-L-D-G-R-G-T-C₁-R-G-COOH
or
(SEQ ID NO: 36)
H₂N-G-R-C₁-T-R-G-D-L-G-R-L-K-K-C₂-Y-P-D-COOH
or
(SEQ ID NO: 37)
H₂N-D-P-Y-C₂-K-K-L-R-G-L-D-G-R-T-C₁-R-G-COOH
or
(SEQ ID NO: 38)
H₂N-G-R-C₁-T-R-G-D-L-G-R-L-K-C₂-Y-P-D-COOH
or
(SEQ ID NO: 39)
H₂N-D-P-Y-C₂-K-L-R-G-L-D-G-R-T-C₁-R-G-COOH

wherein
C₁ and C₂ form a disulfide bond.

In the peptides of the present invention, preferably, Z₁ is any amino acid, preferably an amino acid selected from the group consisting of F, M, A, V, I, L, H, Y, W, T, Q, S, and N, more preferably F, H, Y, W, T and S, most preferably H or T, or not present.

In the peptides of the present invention, preferably, Z₂ is any amino acid, preferably an amino acid selected from the group consisting of F, M, A, V, I, L, H, Y and W, more preferably F, H, Y and W, most preferably H, or not present.

It is further preferred that the peptides of the present invention form multimers, more preferably dimers, trimers, tetramers, pentamers or hexamers, most preferably trimers. The peptides of the multimers may be linked covalently or non-covalently, preferably covalently. Preferably, the peptides are not directly linked to each other but are linked to each other but through a linker group. The multimers of the invention may be further linked to one, two or more therapeutic agents, or an imaging agents. It is further preferred that the peptides of the multimers are covalently linked via a peptide linker, which may be further linked to one, two or more therapeutic agents, or imaging agents. The peptide linker is preferably comprised of 3 to 6 amino acids, more preferably 4 or 5 amino acids, most preferably 5 amino acids. Preferably, the peptide linker is a S and C rich linker, preferably comprising up to 80% S and C. Preferably, the peptide linker has the sequence GSGSK (SEQ ID NO: 40). Preferably, the monomeric peptides of the invention are linked to form a linear multimer. Preferably, a linear trimeric peptide of the present invention has SEQ ID NO: 41 (R₁-HN-G-R-C₁-T-G-R-G-D-L-G-R-L-C₂-Y-P-D-G-S-G-S-K-G -R-C₁-T-G-R-G-D-L-G-R-L-C₂-Y-P-D-G-S-G-S-K-G-R-C₁-T-G-R-G-D-L-G-R-L-C₂-Y-P-D-COO-R₂).

It is further preferred that the monomeric peptides of the invention are linked to form a branched multimer. Preferably, a branched trimeric peptide of the present invention has the formula (XXVII). Preferably, C₁ and C₂ of each monomeric peptide unit of the branched multimer form disulfide bonds.

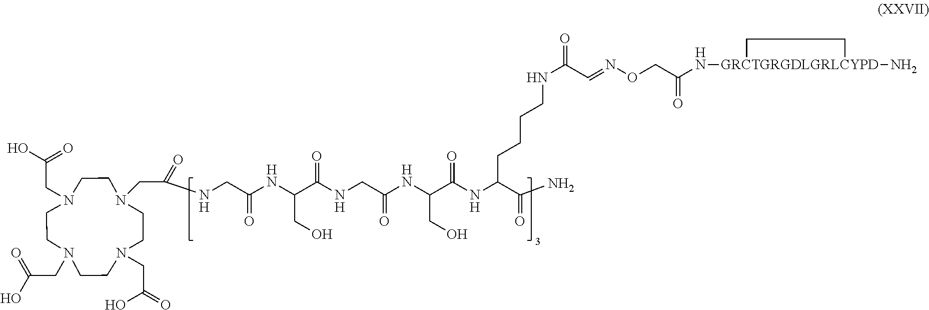

The terms “amino acid” or “any amino acid” as used in the present invention refer to the 20 proteinogenic amino acids encoded by the universal genetic code as well as non-naturally occurring amino acids that have similar charge and size as the proteinogenic amino acids and further to selenocysteine and pyrrolysine. Accordingly, the amino acids of the present invention may preferably be selected from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, selenocysteine and pyrrolysine. For the amino acids of the present invention three-letter symbols and one-letter symbols are used according to the IUPAC “Nomenclature and Symbolism for Amino Acids and Peptides”

Preferably, the polypeptide of the present invention has a total length of at most 24 amino acids, more preferably at most 22 amino acids, more preferably at most 20 amino acids, more preferably at most 18 amino acids, most preferably at most 16 amino acids.

Preferably the number of amino acids that are positioned between the two Cys-residues in formulas (I) to (IV) and further (IX) to (XIV) varies between 8 to 16, more preferably 9 to 15, more preferably 9 to 13 and most preferably 9 to 12.

The polypeptides of the present invention are characterized by their ability to bind to the integrin receptor ITGαᵥβ₆. Preferably, the binding affinity of the polypeptides of the present invention to ITG aᵥ13₆ is at least 50% of the affinity of the polypeptide with the amino acid sequence according to SEQ ID NO: 1. Preferably, the binding affinity of the present invention to ITG aᵥ13₆ is at least 60%, at least 60%, at least 70%, at least 80%, at least 90%, at least 95%, at least 99% of the affinity of the polypeptide with the amino acid sequence according to SEQ ID NO: 1.

The polypeptides of the present invention preferably have a K_{D} of binding to ITGαᵥβ₆.of less than 1×10⁻⁶ M, more preferably of less than 1×10⁻⁷ M, more preferably of less than m 5×10⁻⁸ M, more preferably of less than m 4×10⁻⁸ M, more preferably of less than m 3×10⁻⁸ M, more preferably of less than m 2×10⁻⁸ M more preferably of less than m 1×10⁻⁸ M, more preferably of less than 5×10⁻⁹ M, more preferably of less than 1×10⁻⁹ M.

The polypeptide of the present invention comprises integrin binding motives RGD and LXXL included in a small rigid peptide scaffold. The peptide scaffold is stabilized by a cysteine bridge resulting in a constrained structure with high proteolytic stability. Due to the exposed nature of the binding motive the construct further shows a high affinity for ITGαᵥβ₆, while being easily accessible and quickly synthesized at low costs.

When chemically synthesizing a polypeptide e.g. by solid phase techniques, generating extremely high yields in each step is of overwhelming importance. Nevertheless, if each coupling step were to have 98% yield, a 18-amino acid peptide would be synthesized in 70% final yield, while a 36-amino acid peptide would be synthesized in 48% final yield, and a 54-amino acid peptide would be synthesized in a 34% final yield.

The term “antibody” as used in the context of the present invention refers to a glycoprotein belonging to the immunoglobulin superfamily; the terms antibody and immunoglobulin are often used interchangeably. An antibody refers to a protein molecule produced by plasma cells and is used by the immune system to identify and neutralize foreign objects such as bacteria and viruses. The antibody recognizes a unique part of the foreign target, its antigen.

The term “antibody fragment” as used herein, refers to one or more fragments of an antibody that retain the ability to specifically bind to an antigen. Examples of binding fragments encompassed within the term “antibody fragment” include a fragment antigen binding (Fab) fragment, a Fab′ fragment, a F(ab′)₂ fragment, a heavy chain antibody, a single-domain antibody (sdAb), a single-chain fragment variable (scFv), a fragment variable (Fv), a V_{H} domain, a V_{L} domain, a single domain antibody, a nanobody, an IgNAR (immunoglobulin new antigen receptor), a di-scFv, a bispecific T-cell engager (BITEs), a dual affinity re-targeting (DART) molecule, a triple body, a diabody, a single-chain diabody, an alternative scaffold protein, and a fusion protein thereof.

The term “diabody” as used within this specification refers to a fusion protein or a bivalent antibody which can bind different antigens. A diabody is composed of two single protein chains which comprise fragments of an antibody, namely variable fragments. Diabodies comprise a heavy chain variable domain (V_{H}) connected to a light-chain variable domain (V_{L}) on the same polypeptide chain (V_{H}-V_{L}, or V_{L}-V_{H}). By using a short peptide connecting the two variable domains, the domains are forced to pair with the complementary domain of another chain and thus, create two antigen-binding sites. Diabodies can target the same (monospecific) or different antigens (bispecific).

A “single domain antibody”, as used in the context of the present invention refers to antibody fragments consisting of a single, monomeric variable domain of an antibody. Simply, they only comprise the monomeric heavy chain variable regions of heavy chain antibodies produced by camelids or cartilaginous fish. Due to their different origins they are also referred to VHH or VNAR (variable new antigen receptor)-fragments. Alternatively, single-domain antibodies can be obtained by monomerization of variable domains of conventional mouse or human antibodies by the use of genetic engineering. They show a molecular mass of approximately 12-15 kDa and thus, are the smallest antibody fragments capable of antigen recognition. Further examples include nanobodies or nanoantibodies.

The term “antibody mimetic” as used within the context of the present invention refers to compounds which can specifically bind antigens, similar to an antibody, but are not structurally related to antibodies. Usually, antibody mimetics are artificial peptides or proteins with a molar mass of about 3 to 20 kDa which comprise one, two or more exposed domains specifically binding to an antigen. Examples include inter alia the LACI-D1 (lipoprotein-associated coagulation inhibitor); affilins, e.g. human-γ B crystalline or human ubiquitin; cystatin; Sac7D from Sulfolobus acidocaldarius; lipocalin and anticalins derived from lipocalins; DARPins (designed ankyrin repeat domains); SH3 domain of Fyn; Kunits domain of protease inhibitors; monobodies, e.g. the 10^{th} type III domain of fibronectin; adnectins: knottins (cysteine knot miniproteins); atrimers; evibodies, e.g. CTLA4-based binders, affibodies, e.g. three-helix bundle from Z-domain of protein A from Staphylococcus aureus; Trans-bodies, e.g. human transferrin; tetranectins, e.g. monomeric or trimeric human C-type lectin domain; microbodies, e.g. trypsin-inhibitor-II; affilins; armadillo repeat proteins. Nucleic acids and small molecules are sometimes considered antibody mimetics as well (aptamers), but not artificial antibodies, antibody fragments and fusion proteins composed from these. Common advantages over antibodies are better solubility, tissue penetration, stability towards heat and enzymes, and comparatively low production costs.

Preferably, the polypeptide of the present invention further comprises at least one compound selected from the group consisting of an imaging agent, a therapeutic agent, a linker group or a linker group linked to a therapeutic agent or an imaging agent, wherein the compound is preferably covalently bound outside the sequence comprised by C₁ and C₂ of the polypeptide, preferably to the N-terminus of the polypeptide.

Preferably, the therapeutic agent comprised in the polypeptide of the present invention is selected from the group consisting of small molecules, biopharmaceuticals, and combinations thereof.

The term “small molecules” as used in the present invention relates to a molecule with a molecular weight of about 1000 Dalton or less, preferably a molecular weight of about 900 Dalton or less, usually derived from total chemical synthesis. Suitably the small molecules of the present invention have a sufficient bioavailability and are capable of binding to a specific biological target.

Preferably, the small molecule is preferably an anti-cancer agent, more preferably selected from the group consisting of precyclophosphamide, methotrexate, 5-fluorouracil, mustine, vincristine, procarbazine, prednisolone, doxorubicin, bleomycin, vinblastine, dacarbazine, vincristine, etoposide, cisplatin, epirubicin, capecitabine, folinic acid, oxaliplatin, and combinations thereof.

The term “biopharmaceutical” as used in the present invention refers to pharmaceutical drug products manufactured in, extracted from, or semisynthesized from biological sources. Different from totally synthesized pharmaceuticals, they include vaccines, blood, blood components, allergenics, somatic cells, gene therapies, tissues, recombinant therapeutic protein, and living cells used in cell therapy. Biologics can be composed of sugars, proteins, or nucleic acids or complex combinations of these substances, or may be living cells or tissues. They or their precursors or components are isolated from living sources, such as human, animal, plant, fungal, or microbial.

Preferably, the biopharmaceutical used in the present invention is a protein, preferably a protein selected from the group consisting of an antibody, an antibody fragment, an antibody-mimetic, a cytokine, in particular an interferon, or an interleukin, and combinations thereof.

It is preferred that the antibody is suitable for the treatment of cancer, preferably selected from the group consisting of alemtuzumab, bevacizumab, ibritumomab tiuxetan, ipilimumab, nivolumab, ofatumumab, rituximab, tositumomab, and combinations thereof.

Preferably, the therapeutic agent further comprises a radioisotope selected from the group consisting of alpha radiation emitting isotopes, gamma radiation emitting isotopes, Auger electron emitting isotopes, X-ray emitting isotopes, such as ¹⁸F, ⁵¹Cr, ⁶⁷Ga, ⁶⁸Ga, ¹¹¹In, ^{99m}Tc, ¹⁴⁰La, ¹⁷⁵Yb, ¹⁵³Sm, ¹⁶⁶_{Ho,} ⁸⁸Y, ⁹⁰Y, ¹⁴⁹Pm, ¹⁷⁷Lu, ⁴⁷Sc, ¹⁴²Pr, ¹⁵⁹Gd, ²¹²Bi, ⁷²As, ⁷²Se, ⁹⁷Ru, ¹⁰⁹Pd, ¹⁰⁵Rh, ^{101m15}Rh, ¹¹⁹Sb, ¹²⁸Ba, ¹²³I, ¹²⁴I, ¹²⁵I, ¹³¹I, ¹⁹⁷Hg, ²¹¹At, ¹⁶⁹Eu, ²⁰³Pb, ²¹²Pb, ⁶⁴Cu, ⁶⁷Cu, ¹⁸⁸Re, ¹⁸⁶Re, ¹⁹⁸Au and ¹⁹⁹Ag.

More preferably, the therapeutic agent further comprises a radioisotope selected from the group consisting of ⁶⁷Cu, ⁹⁰Y, ¹³¹Lu, ¹⁸⁶Re, ¹⁸⁸Re, ²¹¹At, ²²⁵Ac, ²¹²Bi, ²¹³Bi, and combinations thereof.

Preferably, the imaging agent is a polydentate chelating agent. A chelating agent is capable of forming two or more separate coordinate bonds with a single central metal atom. Preferably, the chelating agent is selected from the group consisting of 1,4,7,10-tetraazacyclododecane-1,4,7,10-tetraacetic acid (DOTA), 1,4,7-triazacyclononane, 1-glutaric acid-4,7 acetic acid (NODAGA), 1,4,7-triazacyclononane -1,4,7-triacetic acid (NOTA), hydrazine-nicotinic acid (HYNIC), mercaptoacetylglycyltriglycine (MAG3), ethylenediaminetetraacetic acid (EDTA), triethylenetetramine (TETA), iminodiacetic acid, Diethylenetriamine-N,N,N′,N′,N″-pentaacetic acid (DTPA) and combinations thereof, wherein 1,4,7,10-tetraazacyclododecane-1,4,7,10-tetraacetic acid (DOTA) is particularly preferred.

Preferably, the imaging agent further comprises a radioisotope selected from the group consisting of alpha radiation emitting isotopes, gamma radiation emitting isotopes, Auger electron emitting isotopes, X-ray emitting isotopes, such as ¹⁸F, ⁵¹Cr, ⁶⁷Ga, ⁶⁸Ga, ¹¹¹In, ^{99m}Tc, ¹⁴⁰La, ¹⁷⁵Yb, ¹⁵³Sm, ¹⁶⁶_{Ho,} ⁸⁸Y, ⁹⁰Y, ¹⁴⁹Pm, ¹⁷⁷Lu, ⁴⁷Sc, ¹⁴²Pr, ¹⁵⁹Gd, ²¹²Bi, ⁷²As, ⁷²Se, ⁹⁷Ru, ¹⁰⁹Pd, ¹⁰⁵Rh, ^{101m15}Rh, ¹¹⁹Sb, ¹²⁸Ba, ¹²³I, ¹²⁴I, ¹²⁵I, ¹³¹I, ¹⁹⁷Hg, ²¹¹At, ¹⁶⁹Eu, ²⁰³Pb, ²¹²Pb, ⁶⁴Cu, ⁶⁷Cu, ¹⁸⁸Re, ¹⁸⁶Re, ¹⁹⁸Au and ¹⁹⁹Ag.

It is further preferred that the polydentate chelating agent further comprises a radioisotope selected from the group consisting of ⁶⁸Ga, ⁶⁷Ga, ^{99m}Tc, ¹¹¹In, ¹²³I, ¹²⁴I, ¹³¹I, ¹⁷⁷Lu, and combinations thereof.

Particularly preferred polydentante chelating agents comprising a radioisotope are selected from the group consisting of ⁶⁸Ga-DOTA, ¹⁷⁷Lu-DOTA and combinations thereof. It if further preferred that these polydentante chelating agents comprising a radioisotope are coupled to the N-terminus of the peptide of the present invention.

In the alternative, it is preferred that the imaging agent is a molecule comprising a radioisotope selected from the group consisting of ¹⁸F, ¹¹C, and combinations thereof.

Preferably, the imaging agent is a molecule comprising a ¹⁸F radioisotope selected from the group consisting of ¹⁸F-nucleosides, ¹⁸F-fluoroarenes, ¹⁸F-monosaccharides, and combinations thereof.

Examples of ¹⁸F-nucleosides include ¹⁸F-fluorothymidine (18F-FLT), 5-⁸F-fluorouracil (5-⁸F-FU), 9-(4-¹⁸F-fluoro-3-[hydroxymethyl]butyl)guanine (¹⁸F-FHBG), 2′-deoxy-2′-[¹⁸F]fluoro-1-D -arabinofuranosyl-adenine, ¹⁸F-FAA, and the like.

Examples of ¹⁸F-fluoroarenes include ¹⁸F-fluorobenzoic acid (¹⁸F-FBA), 4-azidophenacyl-¹⁸F -fluoride (¹⁸F-APF), and the like.

Examples of, ¹⁸F-monosaccharides include 2-deoxy-2-¹⁸F-fluoro-β-D-glucose (¹⁸F-FDG) and the like.

Preferably, the imaging agent is a molecule comprising a ¹¹C radioisotope selected from the group consisting of ¹¹C-metomidate, N-methyl-¹¹C-vorozole, ¹¹C-glucose, and combinations thereof

It is further preferably, that a radioisotope is directly introduced in one or more of the amino acids, preferably the sidechains of the amino acids, of the polypeptide of the invention. Accordingly, the polypeptide of the invention may comprise a radioisotope selected from the group consisting of ¹¹C, ¹⁸F, ¹²⁵I, ¹³¹I, and combinations thereof. It is particularly preferred that the radionuclides ¹²⁵I and/or ¹³¹I are introduced into the side chain of a tyrosine comprised in the polypeptide of the present invention.

It is further preferred that imaging agent is a monodentate complex-forming molecule. The monodentate complex-forming molecule is capable of binding to a central atom, preferably a metal atom, in a coordination complex. The monodentate complex-forming molecule preferably selected from the group consisting of 5-fluorouracil, diacetyl-bis(N4-methyl-tiosemicarbazone (ATSM), pyrovaldehyde -bis(N4-methyltiosemicarbazone (PTSM), citrate, and combinations thereof.

Preferably, the monodentate complex-forming molecule further comprises a radioisotope selected from the group consisting of ⁶⁴Cu, ⁶⁸Ga, and combinations thereof.

The linker group is preferably selected from the group consisting of alkyl, cycloalkyl, heteroalkyl, arylalkyl, alkoxy, aryloxy, amino, amide, silyl, alkenyl, cycloalkenyl, heteroalkenyl, alkynyl, aryl, heteroaryl, acyl, carbonyl, carboxylic acid, ether, ester, nitrile, isonitrile, sulfanyl, sulfinyl, sulfonyl, phosphino, and combinations thereof. The person skilled in the art is able to select the suitable linker(s) depending on the respective application.

Preferably, the therapeutic agent and/or imaging agent are coupled to the respective amino acid of the polypeptide of the present invention by using an activated ester. In particular, in case of therapeutic agent and/or imaging agent to the amino acid having an amino group in a side chain this method can be used. Alternatively, the following coupling methods can be used in order to couple therapeutic agent and/or imaging agent to the respective amino acid, which are shortly summarized. The specific reaction conditions for achieving a coupling of a therapeutic agent and/or imaging agent to an amino acid with or without a linker can be easily determined by the person skilled in the art:

Formation of amides by the reaction of an amine and activated carboxylic acids, preferably NHS-esters or carbodiimides; a carbodiimide is a complete crosslinker that facilitates the direct conjugation of carboxyls to primary amine NHS esters are reactive groups formed by carbodiimide-activation of molecules containing carboxylate groups

Disulfide linkage using two thiols or one thiol that specifically reacts with pyridyl disulfides; Pyridyl disulfides react with sulfhydryl groups over a broad pH range (the optimum is pH 4-5) to form disulfide bonds. During the reaction, a disulfide exchange occurs between the molecule's SH-group and the 2-pyridyldithiol group. As a result, pyridine-2-thione is released.

Thioether formation using maleimides or haloacetyls and a thiol component; Haloacetyls react with sulfhydryl groups at physiologic pH. The reaction of the iodoacetyl group proceeds by nucleophilic substitution of iodine with a sulfur atom from a sulfhydryl group to result a stable thioether linkage. The maleimide group reacts specifically with sulfhydryl groups when the pH of the reaction mixture is between pH 6.5 and 7.5 and forms a stable thioether linkage that is not reversible.

Amidine formation using an imidoester and an amine; Imidoester crosslinkers react rapidly with amines at alkaline pH but have short half-lives. As the pH becomes more alkaline, the half life and reactivity with amines increases; therefore, crosslinking is more efficient when performed at pH 10 than at pH 8. Reaction conditions below pH 10 may result in side reactions, although amidine formation is favoured between pH 8-10

Hydrazide linkage using carbonyls (e.g. aldehydes) and hydrazides; Carbonyls (aldehydes and ketones) react with hydrazides and amines at pH 5-7. Carbonyls do not readily exist in proteins; however, mild oxidation of sugar glycols using sodium meta-periodate converts vicinal hydroxyls to aldehydes or ketones. Subsequent reaction with hydrazides results in the formation of a hydrazone bond.

Amine linkage using carbonyls and amines under reductive conditions; Reductive amination (also known as reductive alkylation) is a form of amination that involves the conversion of a carbonyl group to an amine via an intermediate imine. The carbonyl group is most commonly a ketone or an aldehyde.

Copper-catalyzed triazole formation using nitriles and azides. The Huisgen-type 1,3-dipolar cycloaddition between an azide and a terminal or internal alkyne is used to give stable 1,2,3-triazoles.

Isothiourea formation using isothiocyanates and amines; the reaction between isothiocyanates and amines, i.e. the E-amino groups of lysine leads to a stable isothiourea bond.

Formation of esters by the reaction of an alcohol and activated carboxylic acids, preferably acid chlorides or carbodiimides; High temperature reaction allows direct reaction between alcohols and carboxylic acids to form stable esters, alternatively the carboxylic acids and be activated under acidic or base catalytic conditions.

Formation of ethers by the reaction of an alcohol and alkyl halides. Haloalkanes are reactive towards nucleophiles. They are polar molecules: the carbon to which the halogen is attached is slightly electropositive where the halogen is slightly electronegative. This results in an electron deficient (electrophilic) carbon which, inevitably, attracts nucleophiles.

The polypeptide of the present invention is suitable for use in medicine, preferably in the treatment of cancer. Preferred cancers to be treated are ITGαᵥβ₆-positive carcinomas.

The polypeptide of the present invention is further suitable for use in the diagnosis and/or evaluation of cancer. The term “evaluation” includes the assessment of a cancer that is diagnosed and that is typically carried out by histologic or imaging methods to determine the treatment strategy, e.g. staging, size, assessment of cancer periphery, involvement of lymph nodes, and near or distant metastasis. Preferred cancers to be diagnosed and/or evaluated are ITGαᵥβ₆-positive carcinomas.

Preferably, the cancer is selected from the group consisting of head and neck squamous cell carcinoma, hypopharynx carcinoma, collangiocarcinoma, breast cancer, brain cancer, pancreatic cancer, ovarian cancer, kidney cancer, bowel cancer, stomach cancer, cervical cancer, thyroid cancer, pancreatic cancer, gastric cancer, lung cancer, colorectal cancer, liver cancer.

### EXAMPLE 1

### Material and Methods

### Patient Samples and Cell Lines

Intraoperatively obtained tissues from HNSCC or brain metastasis were either snap frozen or in part used to establish tumor cell cultures HNO97, HNO407, HNO199, HNO210, HNO258 as described in the prior art. Cell line authentification was performed by the German Collection of Microorganisms and Cell Cultures (DMSZ). Written informed consent was obtained from all patients according to the research proposals approved by the Institutional Review Board at the Medical Faculty of the University of Heidelberg. The human carcinoma cell lines UM-UC-5 (bladder), LUDLU-1 (lung), and MCF-7 (breast), liposarcoma cell line SW872 were purchased from European Collection of Authenticated Cell Cultures (ECACC), the colorectal adenocarcinoma cell line HT29 was obtained from ATCC, and the authentification was performed by Multiplexion (Friedrichshafen). The breast carcinoma cell line T47D and the primary human gingival mucosa keratinocyte cells HPV16GM were kindly provided by Prof. Dr. Stefan Dithel (TU Braunschweig) and by Prof. Dr. Pascal Tomakidi (Freiburg), respectively. HPV16GM cells were cultured in Keratinocyte Growth Medium 2 (Promocell). All HNSCC cell lines as well as the cell lines T47D, MCF-7, LUDLU-1 and SW872 were cultured in RPMI 1640 medium (Gibco) supplemented with 10% FCS. UM-UC-5 and HT29 were cultured in EDSS medium (Gibco) supplemented with 10% FCS, 1% L-glutamine and 1% non-essential amino acids and DMEM high Glucose medium supplemented with 1 mM sodium pyruvate and 10% FCS, respectively. All cell lines were negative for Mycoplasma and maintained at 37° C. and 5% CO₂.

### Membrane Protein Isolation and Protein Fractionation

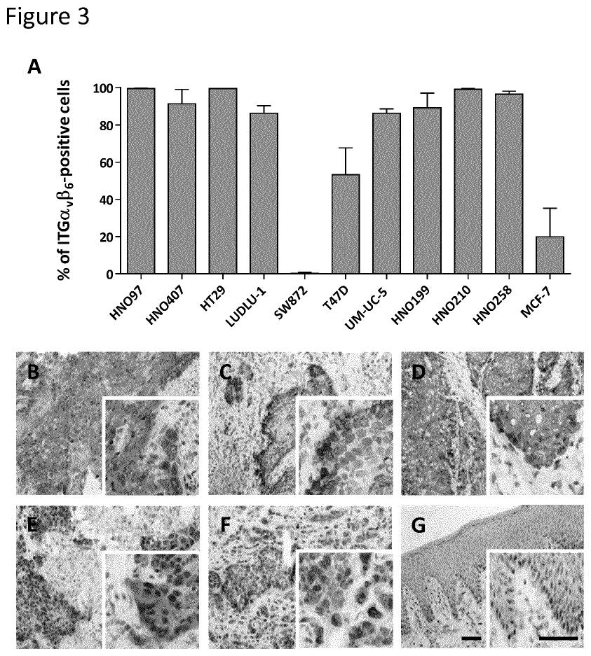

To harvest membrane proteins HNO97 and HPV16GM cells were grown to 90% confluence in multilayer flasks (HYPERFlask™, Corning) and detached by pre-incubation with PBS/0.5% EDTA and subsequent 0.025% trypsin treatment. Membrane proteins were extracted according to an ultracentrifugation-based protocol. Briefly, cells were washed, pelleted and lysed by mechanical dissociation using a dounce tissue grinder (Wheaton) with 4 mL of lysis buffer (50 nM TRIS (pH 7.3), 250 mM sucrose, 2 mM EDTA, 2 mM protease inhibitor). Lysates were centrifuged (2800×g, 4° C., 20 min) and supernatants including cytosolic and membrane proteins were collected. Next, ultracentrifugation (>100.000×g/1 hour/4° C.) (Sorvall™ Dicovery 90SE, Hitachi) was used to separate cytosolic and membrane proteins. Membrane fraction purity was verified by western blot assessing epidermal growth factor receptor (EGFR) expression (FIG. 2). Pellets containing the membrane fractions were resuspended in PF2D start buffer (Beckman Coulter). Protein concentrations were determined applying the Micro BCA™ Protein Assay Kit (Thermo Fischer Scientific) according to manufacturer's protocol.

Fractionation of membrane proteins was conducted by the liquid chromatography system ProteomeLab™ as described before (Beckhove et al., J Clin Investig, 2010, 120, 2230-429; Ménoret et al., J Nucl Med, 2013, 54, 2146-52). Briefly, a total of 2.5 mg protein was loaded on the first dimension (1D) chroma to focusing column according to manufacturer's protocol (Beckman Coulter). Proteins were separated by isoelectric focusing and fractions were collected at 0.3 pH intervals during the pH gradient. In the second dimension (2D) 200 μL of each 1D fraction was loaded onto the 2D reversed phase column (heated to 50° C.) for further fractionation. Column-bound proteins were resolved by a 30 min linear gradient from solvent A (0.1% aqueous trifluoracetic acid (TFA)) to solvent B (0.08% TFA acid in acetonitrile), respectively. Eluted proteins were detected at 214 nm and collected. UV absorbance data were further analyzed using the ProteoVue and DeltaVue software (Beckman Coulter). Protein profiles of tumor cells and human keratinocytes (HPV16GM) were compared regarding the appearance of distinct tumor-specific peaks. Corresponding fractions were selected and pooled (Billecke et al., Molecular & Cellular Proteomics, 2006, 5, 35-42) and depending on the protein concentration concentrated using a vacuum concentrator (Bachhofer Savant).

### Western Blot

For Western blot analyses of the HNO97 protein fractions nucleus, cytosolic and membranous lysates were separated by SDS/PAGE and transferred to a PVDF Membrane (Roche). The membrane was blocked with 5% wt/vol milk powder and a rabbit anti-human EGFR antibody (Abcam, ab2430, 0.4 μg/mL) was applied. After washing, the membrane was incubated with either HPR-conjugated anti-rabbit secondary antibody (Abcam, ab97051, 0.05 μg/mL) or HRP-conjugated β-Actin antibody (Abcam, ab49900, 0.06 μg/mL). Detection of specific EGFR or β-actin bands was carried out by enhanced chemiluminescence (GE Healthcare).

### Generation of the SFTI8Ph Library

The combinatorial sunflower trypsin inhibitor 1-based phage display (SFTI8Ph) library based on the sunflower trypsin inhibitor (SFTI) 1 scaffold structure was constructed by PCR. To this end, 8 amino acids except cysteine were randomly inserted between Thr4 and Cysll in the binding loop of SFTI-1. The following oligonucleotides were used as templates for the PCR reaction: 5′-TTACTCGCTCCATGGGCGGCAGGTGTACTNNN NNN NNN NNN NNN NNN NNN NNN TGTTATCCCGAT-3′ (SFTI8Ph forward; NNN=timer encoding for one variable amino acid; Ella Biotech) (SEQ ID NO: 12) and 5′ -ATAATCTTGCGGCCGCACCGCCACCTGCTGCA TCGGGATAACA-3′ (SFTIPh reverse; Eurofins MWG Operon) (SEQ ID NO: 13). With regard to the ¹²⁵I-labeling of peptides the triplet TTT was exchanged by TAT to achieve a substitution of phenylalanine¹² to tyrosine. The PCR product was cloned in frame into the surface expression phagemid vector pSEX81 (Progen) and single clones were sequenced (GATC Biotech) to control the diversity of the library. For the preparation of phages presenting the peptides (theoretical diversity of 10⁹) fused to the pIII coat protein on their surface the phagemid vector was transformed in XL1-Blue bacteria. After incubation of the bacteria and amplification of the phagemid in LB medium (20% Glucose, Ampicillin (100 μg/mL)) for 1 hour at 37° C. 100 μL M13KO7 helper phage (Invitrogen; 18311-019) was added to the suspension for packaging of the phagmid overnight in LB medium (Kanamycin (50 μg/mL)). The suspension was centrifuged for 20 min at 4° C. and 5000 rpm and the supernatant was added to ice-cold polyethylene-glycol solution for 30 min on ice for precipitation of the phages. After centrifugation for 15 min, at 4° C. and 13000 rpm the phages were resuspended in 1 mL PBS and stored at 4° C.

### Selection of Tumor Cell-Binding Peptides

For the selection of HNSCC-specific peptides an alternating biopanning using the SFTI8Ph library (see above) against HNO97 cells and the corresponding protein fraction was performed. Initially, 1×109 phages were incubated for 1 hour with HNO97 grown to 90% confluence. Unbound phages were removed by washing steps and the cells were lysed with 1% Triton X-100 solution. Phages isolated from cell lysate were amplified and packaged in XL1 blue bacteria overnight and precipitated in polyethylenglycole solution. Subsequently, the phages were exposed alternatingly to HNO97 cells and HNO97 protein fractions (100 nM) in 96-well plates for 1 hour. After 5 PBS washing steps phages were eluted in 100 μL Glycin/HCl (pH 2.2) per well, neutralized by 15 μL Tris-HCl (pH 9.1) and amplified in XL1 blue bacteria. For titration, the phages were diluted (10-2, 10-4, 10-6) and grown on agar plates. Twelve selection rounds were performed followed by single-stranded DNA isolation of 16 clones (QIApreo Spin M13 Kit; Qiagen). DNA sequencing (GATC Biotech) allowed for the identification of the corresponding peptides which were synthesized using standard Fmoc/tBu chemistry (see below).

### Peptide Synthesis Using Fmoc/tBU Chemistry

The peptides and their modifications were obtained by solid-phase peptide synthesis using standard Fmoc/tBu chemistry on an Applied Biosystems ABI 433A synthesizer. Cleavage of protection groups and resin was performed with 2.5% water and 2.5% triisopropylsilane in TFA for 1 hour. Deprotected peptides were precipitated in cold diethyl ether. For disulfide bridge formation peptides were dissolved in 80% acetic acid in water with a concentration of 1 mg/mL. Subsequently, the solution was poured into the same volume of a 0.25 mg/ml solution of iodine in acetic acid. The reaction was quenched after 5 min with a hundredth volume of a 20% aqueous ascorbic acid solution. Solvents were evaporated and the residuals dissolved in 50% acetonitrile in water supplemented with 0.1% TFA. Purification was achieved by semi preparative reversed-phase high-performance liquid chromatography (RP-HPLC) on a Waters XBridgeBEH130 PREP C18 column (5 μm, 19×150 mm). Analyses were performed on an Agilent 1100 HPLC system using a Chromolith Performance RP-C18e column (100×3 mm). As eluents, 0.1% TFA in water (eluent A) and 0.1% TFA in acetonitrile (eluent B) were used. Conditions: linear gradient from 0% to 100% B within 5 min with a flow rate of 2 mL/min; UV absorbance λ=214 nm. The identity of the peptides was verified by HPLC-MS analysis on an Agilent 1200 HPLC system equipped with a Waters Hypersil Gold™ aq (200×2.1 mm) coupled to an Exactive mass spectrometer (Thermo Fisher Scientific).

### Labeling of Peptides with ¹²⁵Iodine, ¹³¹Iodine, ⁶⁸Gallium or ¹⁷⁷Lutetium

Radiolabeling with ¹²⁵I and ¹³¹was conducted at the tyrosine moiety of the peptides. A 1 mM stock solution of the respective peptide in water was prepared. Labeling was performed by the chloramine-T method (35). Radiolabeled peptides were purified by analytical HPLC using a Chromolith Performance RP-18e-column, 100×4.6 mm. DOTA was coupled on solid phase via its mono-p-Nitrophenyol-ester in NMP and 4 equivalents DIPEA. Cleavage of protection groups and resin was performed with 2.5% water and 2.5% triisopropylsilane in TFA for 1.5 hour.

⁶⁸Ga was eluted with 0.6 M aqueous HCl from a ⁶⁸Ge/⁶⁸Ga generator (IDB Holland). 5 μL of a 1 mM solution of the respective peptide in 300 μL aqueous 2.5 M sodium acetate, 10 μL saturated ascorbic acid, and 1 ml of ⁶⁸Ga-generator eluate was heated at 95° C. for 15 min at pH 3.6. Purification was performed with a Chromabond C18 ec SPE cartridge (Macherey -Nagel). The reaction mixture was loaded onto the cartridge and free activity separated by washing the cartridge with 0.9% NaCl. Radiolabeled peptides were eluted with 70% aqueous ethanol. Solvents were evaporated and residual peptides dissolved in appropriate buffer.

50 MBq ¹⁷⁷Lu chloride (ITG Isotope Technologies Garching) in 100 μL 0.4 M sodium acetate buffer pH 5 was added to 1 μL of a 1 mM peptide solution. The reaction mixture was heated at 90° C. for 15 min and purified with a SPE cartridge as described above.

### In Vitro Binding Experiments

For binding studies, 2.5×10⁵ to 4×10⁵ cells were seeded in 6-well plates and cultivated for 48 hours. Cells were incubated for 60 min with 1 mL serum-free medium containing the ¹²⁵I-labeled peptide (see above). For competition experiments, cells were simultaneously exposed to unlabeled (10-4 M to 10-10 M) and ¹²⁵I-labeled peptides. After three washing steps with PBS (pH 7.4) the cells were lysed with 0.5 mL of 0.3 M NaOH. Experiments concerning the internalization, kinetics and efflux were additionally performed with ¹⁷⁷Lu-DOTA-SFITGv6. To evaluate kinetics and efflux HNO97 cells were incubated for different time intervals (10 min to 480 min) and 60 min, respectively, with ¹⁷⁷Lu-DOTA-SFITGv6 and lysed as described. To continue with the efflux experiment radioactive medium was replaced by non-radioactive medium and cells were incubated again for 60 to 240 min before measuring radioactivity of the cell lysates and the medium. For internalization experiments the cells were exposed to ¹⁷⁷Lu-DOTA-SFITGv6 for different time intervals (10 to 240 min) at 37° C. and 4° C. After three washing steps with PBS the cells were incubated with 1 mL of glycine-HCl 50 mmol/L in PBS (pH 2.8) for 10 min at RT to remove the surface bound activity. Then, cells were washed with 3 mL of ice-cold PBS and lysed as described. Radioactivity was determined in a gamma counter and calculated as percentage of the applied dose per 1×106 cells. Experiments were performed three times, and three repetitions per independent experiment were acquired.

### Surface Plasmon Resonance Assay (SPR)

Assessment of the binding affinity was performed on a BiaCore X100 (GE Healthcare). To avoid denaturation of the heterodimer during amide activation ITGαᵥβ₆ and ITGαᵥβ₃, respectively, were used as analytes and SFITGv6 was immobilized at its N-terminus on a C1 sensor Chip (BR-1005-35, GE Healthcare) using a manual amine coupling protocol. Briefly, after activation of the sensor chip surface with a solution of EDC 0.4 M/NHS 0.1 M for 5 min a 100 μM peptide dissolved in HBS-EP running buffer (0.1 M HEPES, 30 mM EDTA, 1.5 M NaCl, 0.5% surfactant P20, pH 7.2) was immobilized with a contact time of 30 sec until a loading level of 18 response units (RU) was obtained. After saturation of the sensor chip surface for 5 min with 1 M ethanolamine/HCl solution (pH 8.4) the binding affinity of the analyte dissolved in HBS-EP running buffer in appropriate concentrations to the ligand was measured (flow rate: 30 μL/min). The SPR-sensogram data were evaluated with the BiaCore evaluation software. The dissociation constant (KD) was determined by a 1:1 Langmuir model fit of the SPR-sensograms.

### Serum Stability Assay

Five MBq of the purified ¹³¹I-radiolabeled peptide was incubated in 300 μL human serum at 37° C. After different time intervals (15 min to 24 hours) 20 μL serum was precipitated with 40 μL acetonitrile. The stability of the labeled peptide in the supernatant was monitored by radio-HPLC at selected time points using a chromolith performance RP18ec column (3 mm×100 mm) equipped with a gamma detector (Packard COBRA™ Auto-Gamma, GMI). Separation condition was a gradient of 0% to 60% aqueous acetonitrile supplemented with 0.1% TFA over 10 min with a flow rate of 2 mL/min.

### Animal Studies

All experiments were conducted in compliance with the German animal protection laws. Eight week old Balb c/c nude mice (Charles River Laboratories) were inoculated subcutaneously at the right shoulder with 5×106 HNO97 cells in BD MatriGel™ (BD Bioscience). Xenografts were grown to a tumor diameter of 10-15 mm. For small animal-PET imaging mice were anesthetized using isoflurane inhalation and injected via tail-vein with 50 MBq (2 nmol) of the ⁶⁸Ga-labeled DOTA-SFITGv6 peptide (see above) solution in 100 μL PBS. Images were recorded on an Inveon small-animal PET scanner (Siemens) using a 60 min emission scan in list mode and a 10 min transmission scan. Images were taken in 3-dimensional (3D) mode and reconstructed iteratively with a fully 3D algorithm from a 256×256 matrix for viewing transaxial, coronal, and sagittal slices of 0.9 mm thickness. Pixel size was 0.38×0.38×0.79 mm3 and transaxial resolution obtained was 0.9 mm. For blocking experiments 100 μL of a 1 mM aqueous solution of SFITGv6 was pre-administered intraperitoneally 30 min before injection of the radiolabeled peptide. Biodistribution studies were performed after administration of 100 μL of a 20 nM ¹⁷⁷Lu-DOTA-SFITGv6 solution (1 MBq) as an intravenous bolus injection into the tail vein of the mice. After different time points (30 min to 6 hours) three animals, respectively, were sacrificed. Peripheral blood, heart, lung, spleen, liver, kidneys, muscle, brain, intestine, and injection site (tail, after intravenous injection only) were collected and weighted. Tissue-associated radioactivity was measured in a gamma counter (Berthold LB951G) and expressed as percentage of the injected dose per gram tissue (% ID/g).

### Histochemical Peptide Staining

Staining with the biotinylated PEG(12)-SFITGv6 peptide was performed on acetone-fixed cryosections (5 μm) of tumor tissues after blocking of unspecific binding using the Avidin/Biotin Blocking Kit (SP-2001, Vector Laboratories). A stock solution of the lyophilized peptide was prepared by dilution in 5% aqueous DMSO. Slices were incubated overnight at 4° C. with 10-5 M peptide concentration in antibody diluent (DAKO). Detection of bound peptide was carried out with the Vectastain Elite ABC Kit (PK-6100, Vector Laboratories) according to manufacturer's protocol. Peptide specificity was ensured by a scrambled (GRD) PEG(12)-SFITGv6 derivate, the somatostatin receptor ligand DOTATOC and negative controls (without peptide or primary antibody) Staining results were assessed by bright field microscopy (BX50) with the SC30 camera and the cell Sense software (all Olympus).

### PET/CT Scans of Tumor Patients

The PET/CT scan was performed 1 and 3 hours post tracer administration with a Biograph mCT Flow™ PET/CT-Scanner (Siemens Medical Solution) using the following parameters: slice thickness of 5 mm, increment of 3-4 mm, soft-tissue reconstruction kernel, care dose Immediately after CT scanning, a whole-body PET was acquired in 3D (matrix 200×200) in FlowMotion™ with 0.7 cm/min. The emission data were corrected for random, scatter and decay. Reconstruction was conducted with an ordered subset expectation maximisation (OSEM) algorithm with 2 iterations/21 subsets and Gauss-filtered to a transaxial resolution of 5 mm at full-width half-maximum (FWHM). Attenuation correction was performed using the low-dose non-enhanced CT data. The quantitative assessment of standardized uptake values (SUV) was done using a region of interest technique.

### Flow Cytometry

The tumor cell lines were analyzed for ITGAVB6 expression by flow cytometry (LSR II, BD Biosciences) using a primary rat monoclonal antibody against human ITGαᵥβ₆ (Abcam, ab97588, 3 μg/mL, 1 hour, 4° C.) followed by incubation with the respective secondary antibody (Alexa Fluor 488 Abcam, ab150153, 1 μg/mL, 30 min, 37° C.). Antibody specificity was ensured by isotype-matched control. Data were analyzed with FlowJo software (TreeStar). Each experiment was repeated at least three times.

### Immunohistochemistry

Immunohistochemical staining was performed on acetone-fixed serial cryosections (5 μm). Primary mouse anti-human ITGαᵥβ₆ antibody (LSBio, LS-C24779, 0.1 mg/mL) was incubated for 1 hour at RT followed by an incubation with a biotin-conjugated anti-mouse secondary antibody (PK-6102, Vectastain) for 30 min at RT. Detection of binding of the secondary antibody was carried out with the Vectastain Elite ABC Kit (PK-6100, Vector Laboratories) according to manufacturer's protocol.

### Results

### Identification, Characterization and Improvement of the HNSCC-Binding Peptide

Employing the SFTI8Ph library for alternate selection rounds on the HNSCC cell line HNO97 and respective PF2D membrane protein fractions 7 out of 16 sequenced phages displaying the peptide sequence FRGDKMQL (SFPF-10) (SEQ ID NO: 15) were selected for further analysis. These 7 out of 16 sequenced clones showed the same motif (FRGDKMQL) (Table 1).

TABLE 1
Peptide sequences of 16 phage-derived clones

| Clone Number | Detected Sequence | SEQ ID NO |
|---|---|---|
| 1 | WWKFMWFQ | 14 |
| 2 | FRGDKMQL* | 15 |
| 3 | FRGDKMQL* | 15 |
| 4 | FRGDKMQL* | 15 |
| 5 | RAWWSHWH | 16 |
| 6 | FRGDKMQL* | 15 |
| 7 | HWFWSKWW | 17 |
| 8 | FRGDKMQL* | 15 |
| 9 | WRWASWFW | 18 |
| 10 | HWFWSKWW | 17 |
| 11 | HWFWSKWW | 17 |
| 12 | WHQNIHSP | 19 |
| 13 | HWFWSKWW | 17 |
| 14 | FRGDKMQL* | 15 |
| 15 | FRGDKMQL* | 15 |
| 16 | HWFWSKWW | 17 |

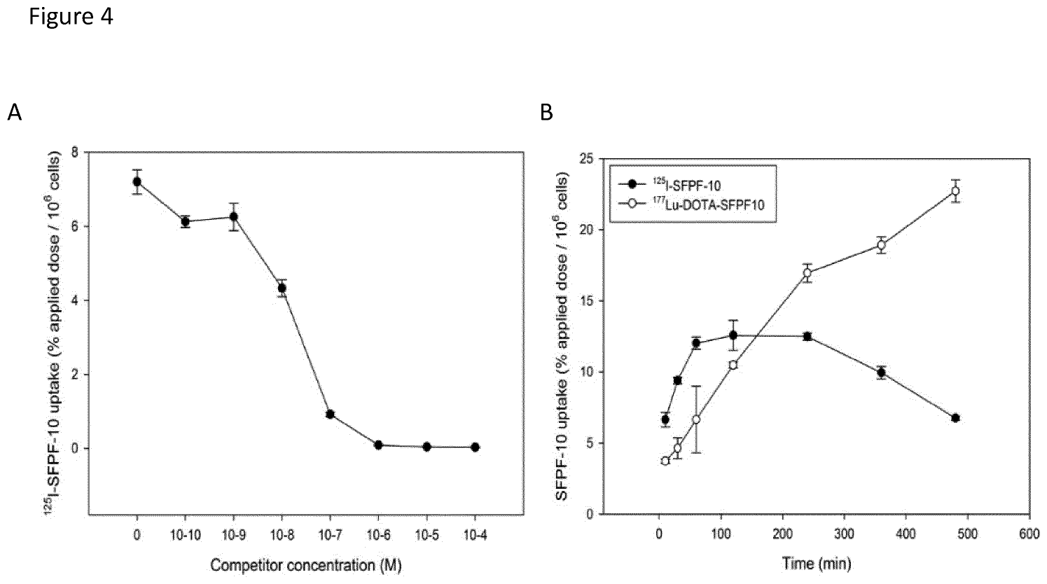

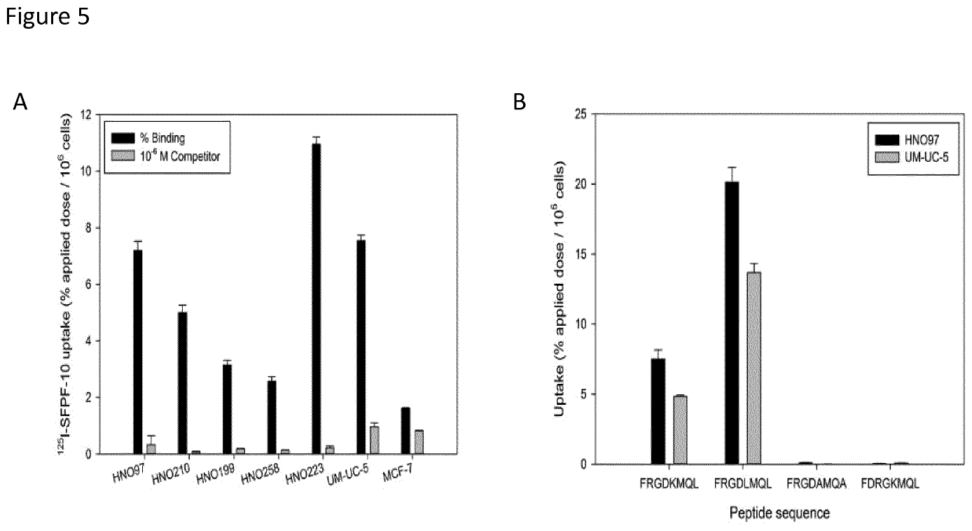

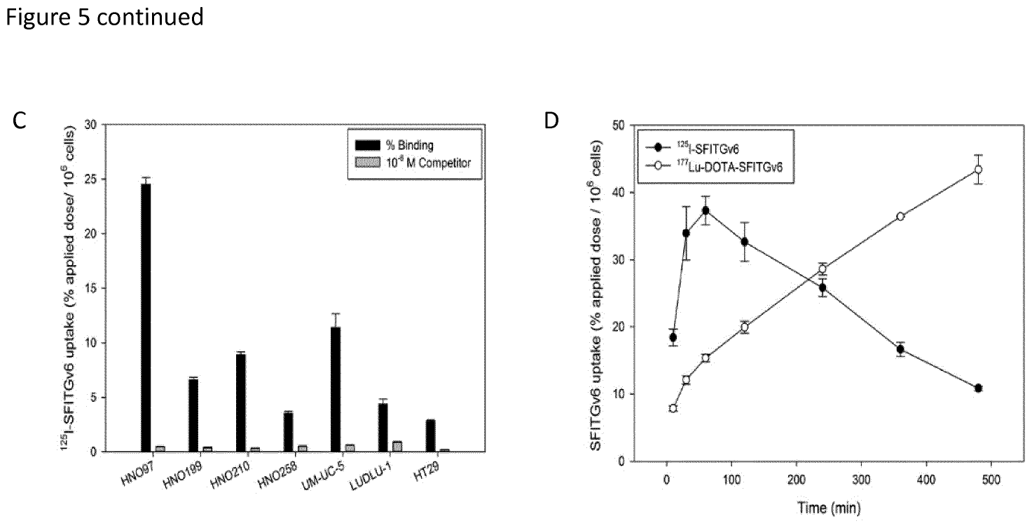

The sequence comprises a RGD and KXXL motif indicating ITGαᵥβ₆-specificity of the peptide. ITGαᵥβ₆-expression could be confirmed by flow cytometry analysis on several squamous cell carcinoma cell lines, including HNSCC (e.g. HNO97; up to 99.8%), bladder cancer (UM-UC-5; up to 88.7%), lung cancer (LUDLU-1; up to 90.9%), and breast cancer (MCF-2; up to 36.1%) (FIG. 3A). In contrast, the liposarcoma cell line SW872 was completely ITGαᵥβ₆-negative. Additionally, ITGαᵥβ₆ expression was assessed in situ applying immunohistochemistry in HNSCC, dysplasia-free normal mucosa tissue as well as in breast and lung cancer-derived brain metastases (FIGS. 3B-G). All tumors showed a strong tumor cell-specific staining, while the tumor-surrounding stromal cells and the epithelial cells of the dysplasia-free mucosa were negative, indicating a tumor-associated ITGαᵥβ₆ expression in squamous cell carcinomas of different origins. Accordingly, in vitro ¹²⁵I-SFPF-10 displayed binding to ITGαᵥβ₆-expressing HNSCC cell lines HNO97 (7.2%) and HNO223 (11%) and to the bladder cancer cell line UM-UC-5 (7.5%) but less binding to the HNSCC cell lines HNO210 (5%), HNO199 (3.1%), HNO258 and (2.6%), and MCF-7 (1.6%) (FIG. 5). In all cancer cell lines tested co-incubation of ¹²⁵I-labeled and unlabeled SFPF-10 (10-6 M) decreased the binding to values below 1% of the applied dose (FIGS. 4 and 5).

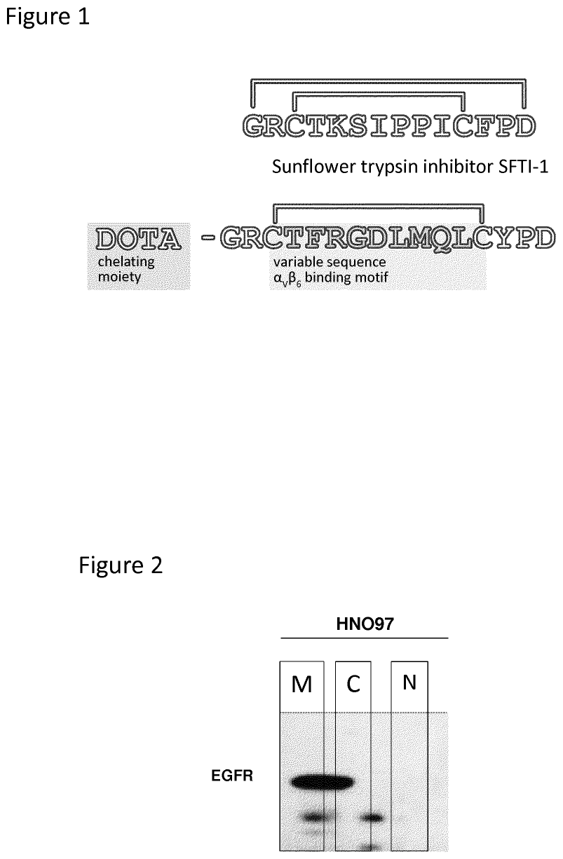

In order to specify the amino acids contributing to the target-specific binding mutations were introduced either into the RGD motif or the adjacent KXXL sequence. As shown in FIG. 5B, both the mutation of RGD to DRG (FDRGKMQL) (SEQ ID NO: 20) and the mutation of KXXL to AXXA (FRGDAMQA) (SEQ ID NO: 21) almost completely abolished (<0.1%) the binding of ¹²⁵I-SFPF-10 to HNO97 and to UM-UC-5 cells, respectively. In contrast, the substitution of lysine (K) to leucine (L) within the initially identified sequence (FRGDKMQL) substantially increased the binding to both cell lines (FIG. 5B). Subsequently, experiments concerning binding and affinity as well as the in vivo application was performed employing the modified peptide SFITGv6 containing the FRGDLMQL sequence (FIG. 1).

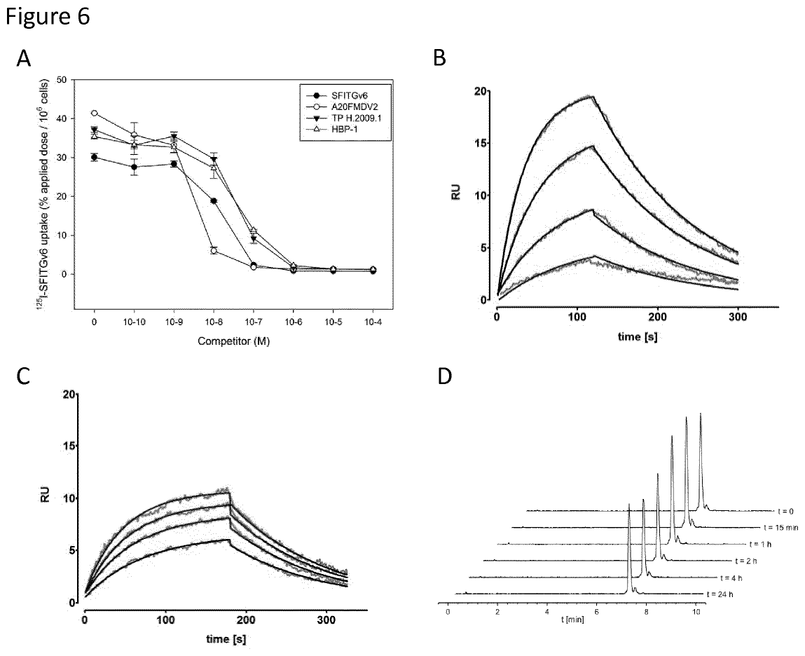

Compared to the originally identified peptide ¹²⁵I-SFITGv6 displayed higher binding to the HNSCC cell lines HNO97 (24.5%), HNO210 (7.1%), HNO199 (6.6%) and HNO258 (3.5%) and to UM-UC-5 cells (10.4%). In addition, binding of ¹²⁵I-SFITGv6 to the carcinoma-derived cell lines LUDLU-1 (4.3%) and to the adenocarcinoma cell line HT29 (2.8%) was measured (FIG. 5C). The specific binding to these cells was reduced to values below 1% of the applied dose by addition of 10-6 M unlabeled peptide as competitor (FIG. 5C). However, in accordance with lower expression levels of ITGαᵥβ₆ (FIG. 3A) less than 2% binding of ¹²⁵I-SFITGv6 to the breast carcinoma cell lines MCF-7 and T47D as well as to the liposarcoma cell line SW872 was measured (FIG. 7A).

Since time-dependent deionization of ¹²⁵1 was expected experiments concerning the kinetics, internalization and efflux of SFITGv6 were additionally performed with the ¹⁷⁷Lu-DOTA-labeled SFITGv6. In fact, binding of the ¹⁷⁷Lu-DOTA-SFITGv6 to HNO97 cells continuously increased to 57.3% within 480 min, whereas the maximal uptake of ¹²⁵I-SFITGv6 (37.3%) was measured after exposure for 60 min followed by a decrease to 14.5% (FIG. 5D). Furthermore, a fast and continuously increasing internalization of ¹⁷⁷Lu-DOTA-SFITGv6 up to 37.5% (FIG. 5E) but maximal internalization of up to 24.1% of ¹²⁵I-labeled SFITGv6 within 60 min was noticed followed by a decrease to 12.6% (FIG. 7B). Finally, the efflux experiment revealed retention of more the 50% of the originally accumulated 177Lu-DOTA-SFITGv6 (FIG. 5F), but less than 5% of ¹²⁵I-SFITGv6 (FIG. 7C) was measured 240 min after the termination of the uptake. These data point to time-dependent deionization of ¹²⁵I-SFITGv6.

### Evaluation of SFPF-10 Specificity for the HNSCC Cell Line HNO97

The specificity of SFPF-10L for ITGαvβ6-expressing HNO97 cells was verified by incubation with the ¹²⁵I-labeled peptide in presence of increasing concentrations of unlabeled peptide as competitor (10⁻¹⁰ M-10⁻⁴ M) and an inhibitory concentration of 50% (IC₅₀) of 18 nM (FIG. 4A) was calculated. Kinetic studies revealed a continuously increasing binding (up to 23%) of ¹⁷⁷Lu-DOTA-SFPF10 to HNO97 cells within 480 min, whereas the maximal uptake of ¹²⁵I-SFPF10 (12.6%) was measured after exposure for 120 min followed by a decrease to 6.7% (FIG. 4B).

### SFITGv6 Shows High Stability and Affinity for ITGαvβ6

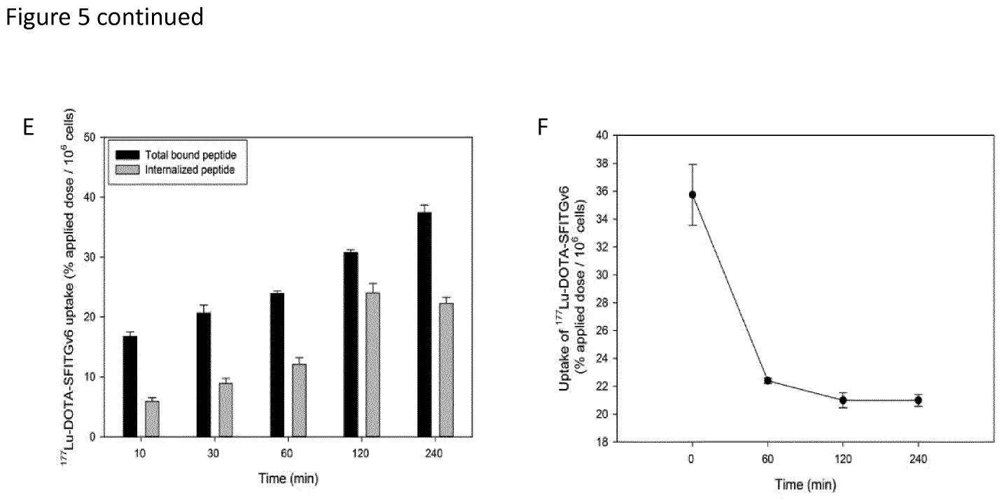

ITGαᵥβ₆-specificity of SFITGv6 was further demonstrated by competition of SFITGv6 binding by already known ITGαvβ6-binding molecules TP H2009.1 (Elayadi et al., Cancer Research, 2007, 67, 5889-95), A2OFMDV2 (Saha et al., J Pathol, 2010, 222, 52-63) and HBP-1 (Nothelfer et al., J of Nucl Med, 2009, 50, 426-34) (FIG. 6A). Like the unlabeled SFITGv6 these peptides competed for the binding of the ¹²⁵I-SFITGv6 to HNO97 cells with IC50 values of 16.15 nM (SFITGv6), 3.2 nM (A20FMDV2), 41 nM (TP H2009.1) and 45.1 nM (HBP-1), respectively (FIG. 6A). In accordance with the IC50 value of SFITGv6 we measured a high affinity (KD=14.8±26.0 nM) of the peptide for ITGαvβ6 (FIG. 6B) using SPR spectroscopy whereas ITGαᵥβ₃ bound to immobilized SFITGv6 with a ten-fold lower affinity (KD=185±0.8 nM) (FIG. 6C). The proteolytic stability of SFITGv6 was determined by radio-HPLC analysis of ¹²⁵I-labeled SFITGv6 after incubation in heparinized human serum (FIG. 6D). Serum aliquots were taken after 0 min, 15 min, 1, 2, 4 and 24 hours, respectively, and ¹²⁵1-SFITGv6 revealed high stability with no degradation over a 24 hours' time period demonstrating the suitability of SFITGv6 for in vivo experiments.

### Evaluation of ¹²⁵I-SFITGv6 Binding to Breast- and Colon Carcinoma Cell Lines and the Internalization and Efflux of ¹²⁵I-SFITGv6 from HNO97 Cells

For the liposarcoma cell line SW872 and the breast carcinoma cell lines MCF-7 and T47D less than 1.5% of the peptide could be detected (FIG. 7A). Maximal internalization of ¹²⁵I-SFITGv6 of up to 24.1% into HNO97 cells was observed after 60 min followed by a decrease to 12.6% (FIG. 7B). 240 min after the termination of the ¹²⁵I-SFITGv6 uptake in HNO97 cells by replacement of the radioactive by non-radioactive medium less than 5% of the radioactivity was detected in the cell lysate (FIG. 7C).

### SFITGv6 Accumulates in ITGαᵥβ₆-Expressing HNO97 Xenograft Tumors

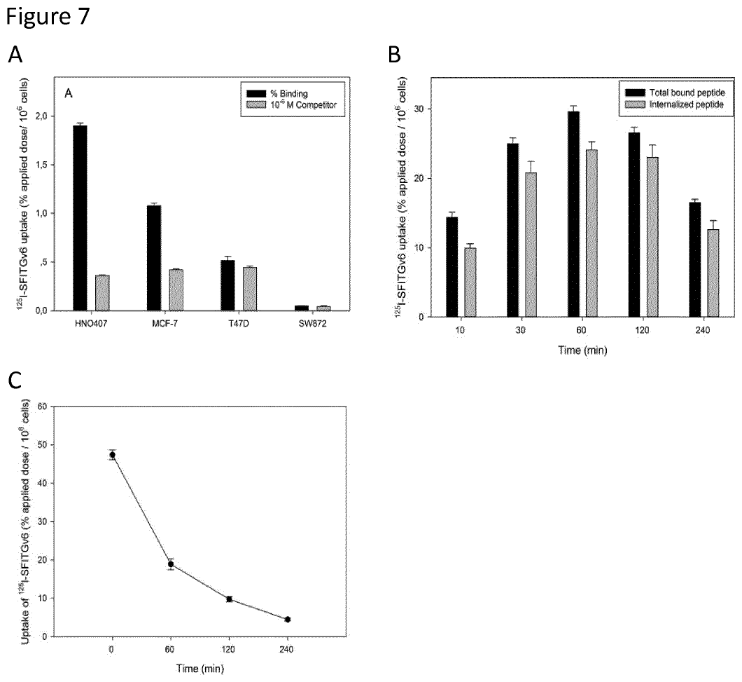

Small-animal PET imaging of Balb/c mice bearing ITGαᵥβ₆-expressing HNO97 xenografts is shown in FIG. 8. Within 20 min after injection of ⁶⁸Ga-DOTA-SFITGv6 radioactivity accumulated in the tumor and was maintained for at least 140 min (FIG. 8A, C). Non-specific activity cleared quickly from the blood within 60 min after injection resulting in a low background and images with good tumor-to-background ratios (FIG. 8A, C). In contrast, no intratumoral accumulation of the peptide was visible after intraperitoneal administration of unlabeled SFITGv6 as competitor 30 min prior to the injection of the radiolabeled compound (FIG. 8B).

To expand on the biodistribution of SFITGv6 the ¹⁷⁷Lu-DOTA-linked peptide was injected intravenously into HNO97 tumor-bearing mice. Radioactivity in individual organs was measured after different time points following injection of the peptide and calculated as % injected dose (ID)/g (FIG. 8D). In the tumor, an activity of more than 6% ID/g was measured 30 min after injection followed by a washout to 2.4% ID/g after 6 hours. A significantly higher uptake of almost 42% ID/g with hardly any clearance was observed in the kidneys, whereas less than 1% ID/g was measured in the blood. Except for the kidneys, the tumor-to-tissue ratios were above one, which is shown in Table 2, and in accordance with the PET results.

TABLE 2
Tumor-to-Tissue Ratio of ¹⁷⁷Lu-DOTA-SFITGv6 in HNO97 xenografts

| Tissue | 30 min | 1 h | 2 h | 4 h | 6 h |
|---|---|---|---|---|---|
| Tumor-to-Blood | 6.89 ± 1.0 | 12.94 ± 0.83 | 59.51 ± 1.04 | 98.86 ± 0.35 | 99.15 ± 0.51 |
| Tumor-to-Heart | 15.06 ± 1.0 | 21.94 ± 0.82 | 48.76 ± 1.04 | 67.50 ± 0.36 | 78.99 ± 0.51 |
| Tumor-to-Lung | 2.29 ± 1.14 | 3.81 ± 0.82 | 9.13 ± 1.05 | 10.32 ± 0.36 | 9.73 ± 0.51 |
| Tumor-to-Spleen | 21.14 ± 1.0 | 27.44 ± 0.82 | 38.39 ± 1.04 | 51.78 ± 0.36 | 35.39 ± 0.51 |
| Tumor-to-Liver | 20.82 ± 1.0 | 21.52 ± 0.82 | 25.78 ± 1.04 | 32.25 ± 0.36 | 17.60 ± 0.51 |
| Tumor-to-Kidney | 0.15 ± 3.73 | 0.12 ± 2.21 | 0.10 ± 3.11 | 0.07 ± 2.69 | 0.06 ± 2.58 |
| Tumor-to-Muscle | 13.89 ± 1.0 | 16.14 ± 0.83 | 22.72 ± 1.04 | 32.28 ± 0.36 | 21.86 ± 0.51 |
| Tumor-to-Intestine | 4.59 ± 1.0 | 3.42 ± 0.83 | 9.44 ± 1.05 | 10.65 ± 0.36 | 7.82 ± 0.51 |
| Tumor-to-Brain | 190.94 ± 1.0 | 198.58 ± 0.82 | 250.35 ± 1.04 | 372.60 ± 0.35 | 424.86 ± 0.51 |

### SFITGv6 Binds Selectively to HNSCC, Brain Metastasis of NSCLC and Breast Cancer In Situ

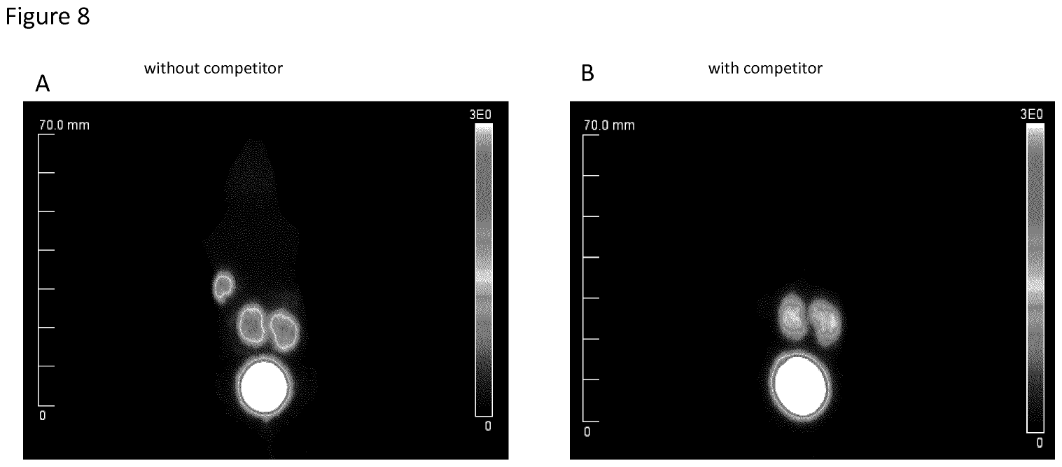

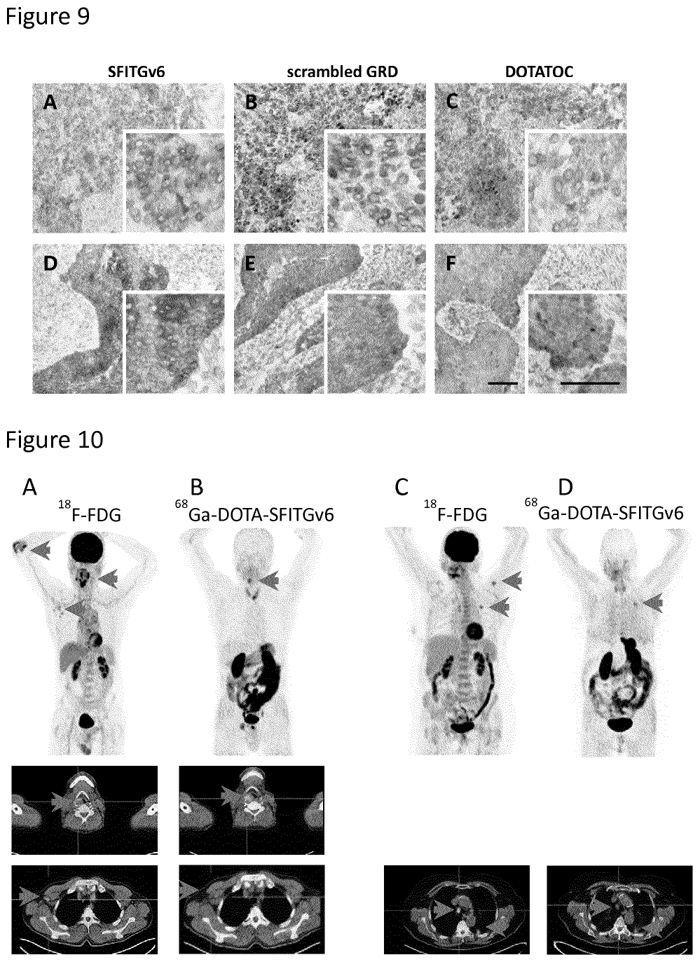

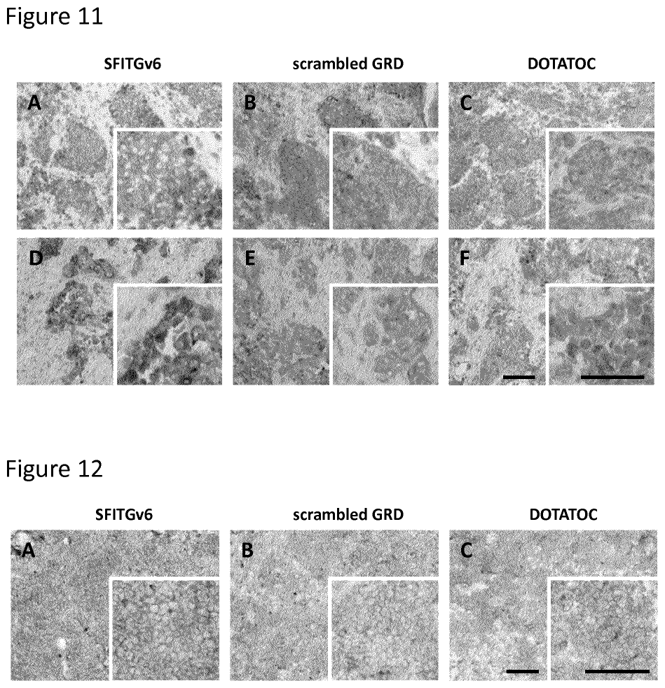

In a next step, tumor cell affinity and intratumoral distribution of SFITGv6 was further assessed by histochemical peptide staining of different carcinomas (HNSCC, NSCLC, breast cancer) using biotin-labeled SFITGv6 (FIG. 9). We observed a strong and homogenous tumor cell-specific binding of SFITGv6, whereas the surrounding tumor stroma was negative (FIG. 9A, D). Moreover, for the negative control peptides containing either the GRD sequence (FIG. 9B, E) or DOTATOC (FIG. 9C, F) we could not observe any specific binding. Additionally, SFITGv6 specificity was assessed on brain metastases derived from breast and NSCLC (FIG. 11). We detected a moderate but distinct tumor cell staining in breast cancer BM (NCH640k) (FIG. 11A), but a very strong tumor-specific staining pattern in lung BM (NCH2407) (FIG. 11D). Inflammation-associated binding was excluded by staining of tumor-free lymph nodes, which did not show any SFITGv6-specificy (FIG. 12A), further corroborating tumor cell specificity of SFITGv6 peptide for HNSCC as well as for further carcinoma.

### ⁶⁸Ga-DOTA-SFITGv6 Accumulates in the Tumor but Not in Inflammatory Lesions of Patients

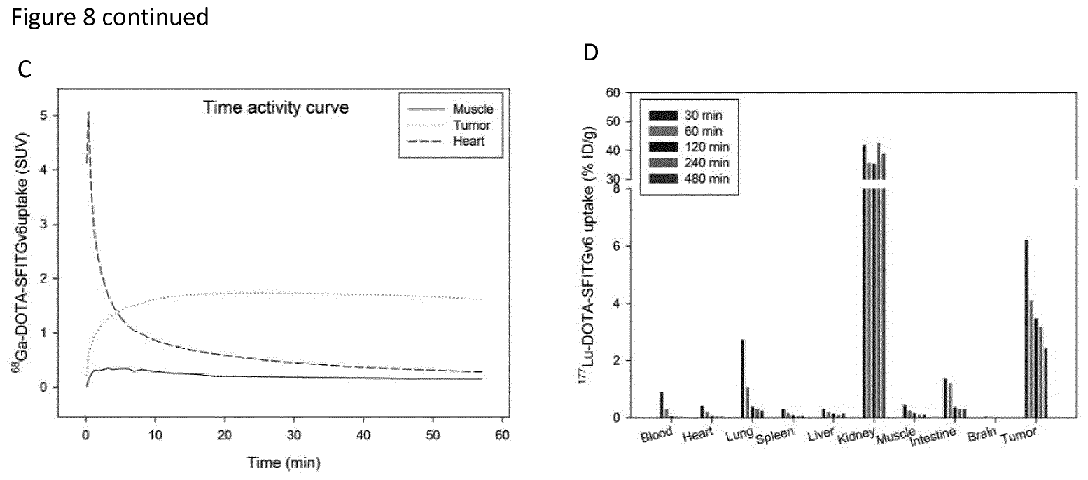

PET/CT scans in a compassionate use setting were performed in two tumor patients after application of ⁶⁸Ga-DOTA-SFITGv6 and ¹⁸F-FDG, respectively (FIG. 10). The first patient suffered from recurrent hypopharynx carcinoma prior to radiation therapy. One and three hours after application of the tracer increasing ¹⁸F-FDG accumulation with time was seen in the recurrent tumor but also in the elbow (FIG. 10A), in several axillary lymph nodes (LN), and the left hilus. In contrast, the PET/CT scan performed after application ⁶⁸Ga-DOTA-SFITGv6 revealed a stable uptake of the tracer only in the tumor (FIG. 10B). We, therefore, considered inflammatory reactions to account for the ¹⁸F-FDG uptake in the axillary LN of this patient. In fact, histological examinations of the lymph nodes excluded the presence of tumor cells in these lesions (data not shown). These results correspond to the negative histochemical staining of carcinoma-free lymph nodes (FIG. 12A). The second patient suffering from NSCLC revealed a time-dependent increase of ¹⁸F-FDG accumulation in the tumor but also uptake of the tracer in the left shoulder and two mediastinal LN which might be due to inflammation (FIG. 10C). ⁶⁸Ga-DOTA-SFITGv6 instead accumulated specifically in the tumor (FIG. 10D) and decreased moderately with time (data not shown). Moreover, the PET/CT scans of both patients revealed a high uptake in the kidneys, the stomach and the bowel and moderate uptake in the thyroid whereas a rather low background was determined in other organs and the blood pool, which is summarized in Tables 3 and 4. Since the bowel activity showed a shift in the localization when comparing the early and the late image a more detailed analysis was done for jejunum, terminal ileum and cecum. In both patients, a time-dependent decrease of the radioactivity in the jejunum and a time-dependent increase of the radioactivity in terminal ileum and cecum were noticed (Tables 3, 4) suggesting that the tracer is secreted into the lumen of the stomach or duodenum/jejunum and is transported intraluminally to the cecum.

TABLE 3
Maximum Standard Uptake values (SUVₘₐₓ) for PET/CT with
¹⁸F-FDG and the ⁶⁸Ga-DOTA-SFITGv6 for one HNSCC patient

SUVₘₐₓ

| Tissue/Organ | Tracer | 1 h | 3 h |
|---|---|---|---|
| Tumor | ¹⁸F-FDG | 13.40 | 16.30 |
| | ⁶⁸Ga-DOTA-SFITGv6 | 3.10 | 3.20 |
| Elbow | ¹⁸F-FDG | 4.00 | n.d |
| | ⁶⁸Ga-DOTA-SFITGv6 | 1.70 | n.d. |
| Axillary LN1 | ¹⁸F-FDG | 3.00 | 3.00 |
| | ⁶⁸Ga-DOTA-SFITGv6 | 0.65 | 0.70 |
| Axillary LN2 | ¹⁸F-FDG | 2.70 | 2.85 |
| | ⁶⁸Ga-DOTA-SFITGv6 | 0.90 | 0.70 |
| Hilar | ¹⁸F-FDG | 3.40 | 3.50 |
| | ⁶⁸Ga-DOTA-SFITGv6 | 1.00 | 1.00 |
| Brain | ⁶⁸Ga-DOTA-SFITGv6 | 0.17 | 0.14 |
| Lung | ⁶⁸Ga-DOTA-SFITGv6 | 0.40 | 0.37 |
| Liver | ⁶⁸Ga-DOTA-SFITGv6 | 1.40 | 1.77 |
| Spleen | ⁶⁸Ga-DOTA-SFITGv6 | 0.88 | 1.06 |
| Kidney (right) | ⁶⁸Ga-DOTA-SFITGv6 | 18.40 | 8.29 |
| Kidney (left) | ⁶⁸Ga-DOTA-SFITGv6 | 29.20 | 12.86 |
| Aorta descendens | ⁶⁸Ga-DOTA-SFITGv6 | 1.00 | 1.07 |
| Jejunum | ⁶⁸Ga-DOTA-SFITGv6 | 7.10 | 4.00 |
| Terminal Ileum | ⁶⁸Ga-DOTA-SFITGv6 | 2.50 | 15.00 |
| Coecum | ⁶⁸Ga-DOTA-SFITGv6 | 3.10 | 10.00 |
| Gallbladder | ⁶⁸Ga-DOTA-SFITGv6 | 1.90 | 1.90 |
| Head of pancreas | ⁶⁸Ga-DOTA-SFITGv6 | 1.40 | 0.96 |
| Corpus of pancreas | ⁶⁸Ga-DOTA-SFITGv6 | 2.20 | 1.37 |
| Stomach | ⁶⁸Ga-DOTA-SFITGv6 | 8.92 | 8.40 |
| Thyroid | ⁶⁸Ga-DOTA-SFITGv6 | 2.55 | 3.00 |
| Bone marrow | ⁶⁸Ga-DOTA-SFITGv6 | 0.63 | 0.65 |
| Parotis (right) | ⁶⁸Ga-DOTA-SFITGv6 | 1.63 | 1.52 |
| Parotis (left) | ⁶⁸Ga-DOTA-SFITGv6 | 1.00 | 0.56 |

¹⁸F-FDG = 314 MBq,
⁶⁸Ga-DOTA-SFITGv6 = 321 MBq,
HNSCC = Head and Neck Squamous Cell Carcinoma,
HYP = Hypopharynx Carcinoma,
LN = Lymphnode,
n.d. = not detectable,
SUV = Standard Uptake Value

TABLE 4
Maximum Standard Uptake values (SUVₘₐₓ) for PET/CT with
¹⁸F-FDG and the ⁶⁸Ga-DOTA-SFITGv6 for one NSCLC patient

SUVₘₐₓ

| Tissue/Organ | Tracer | 1 h | 3 h |
|---|---|---|---|
| Tumor | ¹⁸F-FDG | 3.90 | 4.65 |
| | ⁶⁸Ga-DOTA-SFITGv6 | 3.40 | 3.10 |
| Shoulder | ¹⁸F-FDG | 3.90 | n.d. |
| | ⁶⁸Ga-DOTA-SFITGv6 | 1.50 | n.d. |
| Mediastinal LN1 | ¹⁸F-FDG | 3.60 | 4.00 |
| | ⁵⁸Ga-DOTA-SFITGv6 | 1.20 | 0.90 |
| Mediastinal LN2 | ¹⁸F-FDG | 3.40 | 3.90 |
| | ⁶⁸Ga-DOTA-SFITGv6 | 1.40 | 1.20 |
| Brain | ⁶⁸Ga-DOTA-SFITGv6 | 0.07 | 0.18 |
| | ⁶⁸Ga-DOTA-SFITGv6 | 1.10 | 0.94 |
| Liver | ⁶⁸Ga-DOTA-SFITGv6 | 1.20 | 1.68 |
| | ⁶⁸Ga-DOTA-SFITGv6 | 1.00 | 0.88 |
| Kidney (right) | ⁶⁸Ga-DOTA-SFITGv6 | 13.50 | 10.60 |
| | ⁶⁸Ga-DOTA-SFITGv6 | 10.40 | 10.50 |
| Aorta descendens | ⁵⁸Ga-DOTA-SFITGv6 | 1.63 | 1.29 |
| | ⁶⁸Ga-DOTA-SFITGv6 | 4.20 | 2.80 |
| Terminal Ileum | ⁶⁸Ga-DOTA-SFITGv6 | 2.20 | 16.70 |
| | ⁶⁸Ga-DOTA-SFITGv6 | 4.20 | 15.60 |
| Gallbladder | ⁶⁸Ga-DOTA-SFITGv6 | 0.56 | 0.52 |
| | ⁶⁸Ga-DOTA-SFITGv6 | 1.89 | 1.56 |
| Corpus of pancreas | ⁶⁸Ga-DOTA-SFITGv6 | 1.28 | 1.40 |
| | ⁶⁸Ga-DOTA-SFITGv6 | 14.80 | 11.90 |
| Thyroid | ⁶⁸Ga-DOTA-SFITGv6 | 3.00 | 2.60 |
| | ⁶⁸Ga-DOTA-SFITGv6 | 0.63 | 0.89 |
| Parotis (right) | ⁶⁸Ga-DOTA-SFITGv6 | 0.80 | 0.85 |
| | ⁶⁸Ga-DOTA-SFITGv6 | 0.78 | 0.96 |

¹⁸F-FDG = 351 MBq,
⁵⁸Ga-DOTA-SFITGv6 = 298 MBq,
HNSCC = Head and Neck Squamous Cell Carcinoma,
HYP = Hypopharynx Carcinoma,
LN = Lymphnode,
n.d. = not detectable,
SUV = Standard Uptake Value

### EXAMPLE 2

### Materials and Methods

### Patient Samples and Cell Lines

HNSCC tumor tissues obtained intraoperatively were snap-frozen and stored at −80° C. or directly used to establish primary tumor cell cultures (HNO97, HNO399, HNO223, HNO210 and HNO199) as described in Example 1. Uniqueness of the established cell cultures was assessed by the German Collection of Microorganisms and Cell Culture (DMSZ). HNSCC cell lines were cultured in in RPMI 1640 medium (Gibco) supplemented with 10% FCS. All cell lines were negative for Mycoplasma and maintained at 37° C. and 5% CO₂. Written informed consent was obtained from all patients according to the research proposals approved by the Institutional Review Board at the Medical Faculty of the University of Heidelberg.

### In Vitro Binding Experiments

For the assessment of the peptide binding capacity in vitro, 2.5-4×10⁵ cells were seeded into 6-well plates and cultivated for 48 h. ¹²⁵I-labeled peptide resuspended in 1 mL serum-free medium was added and incubated for either 10 min or 60 min with or without different concentrations of unlabeled peptide (10⁻⁴-10⁻¹¹ M). Afterwards, cell monolayers were washed three times with 1 mL phosphate-buffered saline (pH 7.4) to remove unbound peptide and the cells were harvested with 1.4 mL lysis buffer (0.3 M NaOH). To evaluate kinetic properties the cells were exposed to ¹⁷⁷Lu-DOTA-labeled peptides for 10, 30, 60, 120, and 240 min, respectively, and lysed as described. For the efflux experiments the cells were incubated with ¹⁷⁷Lu-DOTA-labeled peptides for 60 min. Thereafter, radioactive medium was replaced by non-radioactive medium and cell monolayers were incubated for additional 60, 120, and 240 min and afterwards lysed as described earlier. The internalization was assessed after exposure of the cells to ¹⁷⁷Lu-DOTA-labeled peptides for 10, 30, 60, 120, and 240 min at 37° C. Unspecifically bound peptide was removed from the surface by incubation with 1 M glycine-HCl solution (pH 2.2) for 10 min. The monolayer cell was rinsed with PBS and cells were lysed as described before. Radioactivity was determined in a y-counter and calculated as percentage of the applied dose per 1×10⁶ cells. Experiments were performed three times, and three repetitions per independent experiment were acquired.

### Surface Plasmon Resonance (SPR) Assay

Assessment of the binding affinity was performed on a BiaCore X100 (GE Healthcare). To avoid denaturation of the heterodimer during amide activation ITGαᵥβ₆ and ITGαᵥβ₃, respectively, were used as analytes and SFITGv6 was immobilized at its N-terminus on a C1 sensor Chip (BR-1005-35, GE Healthcare) using a manual amine coupling protocol. Briefly, after activation of the sensor chip surface with a solution of EDC 0.4 M/NHS 0.1 M for 5 min a 100 μM SFLAP3 dissolved in HBS-EP running buffer (0.1 M HEPES, 30 mM EDTA, 1.5 M NaCl, 0.5% surfactant P20, pH 7.2) was immobilized with a contact time of 30 sec to obtain a loading level of 12 response units (RU). After saturation of the sensor chip surface for 5 min with 1 M ethanolamine/HCl solution (pH 8.4) the binding affinity of the ITGαᵥβ₆ and ITGαᵥβ₃, respectively, dissolved in HBS-EP running buffer in appropriate concentrations (1-50 μg/mL) to the ligand was measured (flow rate: 30 μL/min). The SPR sensogram data were evaluated with the BiaCore evaluation software. The dissociation constant (K_{D}) was determined by a 1:1 Langmuir model fit of the SPR sensograms.

### Animal Experiments

All animal experiments were conducted in compliance with the German animal protection laws. For in vivo small animal PET imaging and organ distribution studies eight-week old Balb/c nude mice (Charles River Laboratories) were inoculated subcutaneously at the right shoulder with a total of 5×10⁶ tumor cells in MatriGel (BD Bioscience). Xenografts were grown until a tumor diameter of 10-15 mm was reached.

For imaging, tumor-bearing mice were anesthetized using isofluorane inhalation and injected into the tail-vein with a 100 μl PBS solution containing ⁶⁸Ga-DOTA-SFLAP3 (HNO97: 37.9 MBq; HNO399: 26 MBq; HNO233: 27 MBq) and ⁶⁸Ga-DOTA-SFITGv6 (HNO97: 30 MBq; HNO399: 34 MBq; HNO 223: 34 MBq). 3-dimensional (3D) PET images of whole mice were captured by the Siemens Inveon PET scanner as described in Example 1. To assess the biodistribution 100 μL of a 20 nM solution (1 MBq) of ¹⁷⁷Lu-DOTA-SFLAP3 and ¹⁷⁷Lu-DOTA-SFITGv6 were administered as an intravenous bolus injection into the tail vein of mice. For each time point (30 min to 6 hours) we sacrificed a total of three animals, and subsequently collected peripheral blood, heart, lung, spleen, liver, kidneys, muscle, brain, and intestine. Tissues were weighed and respective radioactivity was measured by a gamma counter (LB951G, Berthold Technologies). Values were expressed as percentage of the injected dose radioactivity (MBq) per gram tissue (% ID/g).

### Immunohistochemical Peptide Staining

Immunohistochemical stainings were performed by use of the biotinylated PEG(12)-labled peptides (SFITGv6, SFLAP3) on 5 μm acetone-fixed tumor tissue cryosections. To prevent background staining we initially applied the Avidin/Biotin Blocking Kit (SP-2001, Vector Laboratories). The lyophilized peptides were diluted in 5% aqueous DMSO preparing a 1 mM Stock solution. The HNSCC cryosections were incubated with 10⁻⁵ M peptide concentration in antibody diluent (DAKO) at 4° C. overnight. To detect bound peptide the Vectastain Elite ABC Kit (PK-6100, Vector Laboratories) was applied according to manufacturer's protocol. Peptide binding specificity was ensured by use of a peptide derivate (scrambled control) and negative controls (without peptide). Due to instability of the peptide binding staining results were assessed on the same day by bright field microscopy (BX50) with the SC30 camera and the cell Sense software (all Olympus).

### PET/CT Scan of HNSCC Tumor Patient

A non-contrast-enhanced PET/CT scan was performed 60 and 180 min after intravenous injection of the ⁶⁸Ga-DOTA-labeled peptide using a SIEMENS-BIOGRAPH mCt Flow™ PET/CT-Scanner (Siemens Medical Solution) and the following parameters: slice thickness of 5 mm, increment of 3-4 mm, soft-tissue reconstruction kernel, Care dose Immediately after CT scanning, a whole body PET was acquired in 3-D (matrix 200×200) in FlowMotion with 0.7 cm/min. The emission data were corrected for randoms, scatter and decay. Reconstruction was conducted with an ordered subset expectation maximisation (OSEM) algorithm with 2 iterations/21 subsets and Gauss-filtered to a transaxial resolution of 5 mm at full-width half-maximum (FWHM). Attenuation correction was performed using the low-dose non-enhanced CT data. The quantitative assessment of tracer accumulation was done using a region of interest technique and using standardized uptake values (SUV).

### Results

### Binding of SFTI Derivates to ITGαᵥβ₆-Expressing HNSCC Cell Lines

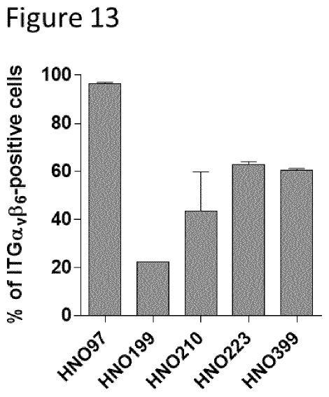

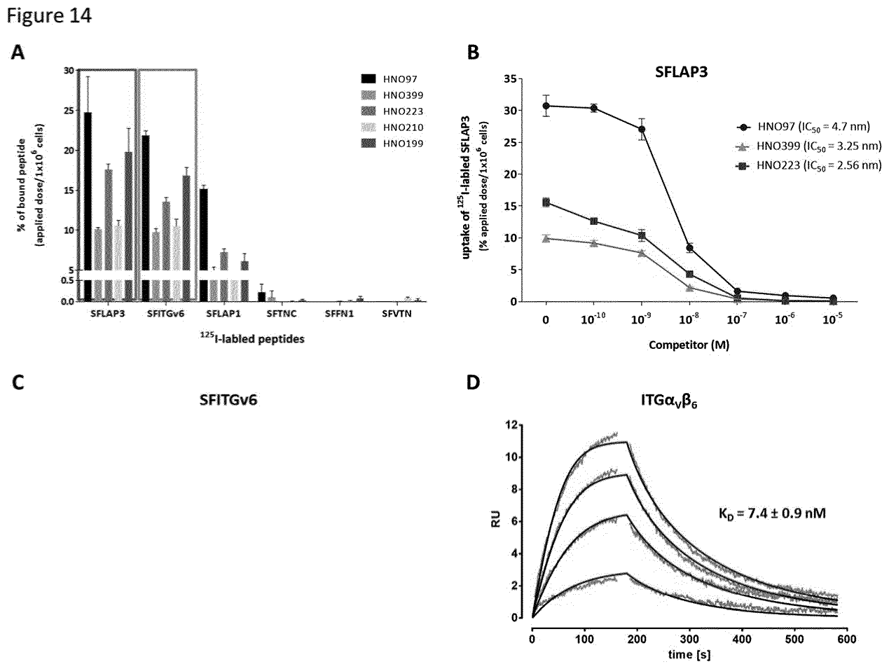

The RGD motif-containing octamers of the natural ITGαᵥβ₆-ligands FN1, TNC, VTN, LAP1 and LAP3 were grafted between Thr4 and Cys11 into the binding loop of the SFTI scaffold (Table 5). First, the binding properties of the ¹²⁵I-labeled SFTI derivates SFFN1, SFTNC, SFVTN, SFLAP1 SFLAP3, and SFITGv6 were compared as described in Example 1 using five primary HNSCC cell lines that differ in their ITGαᵥβ₆-expression as assessed by FACS analysis (FIG. 13). As shown in FIG. 14A, ¹²⁵I-SFLAP3 (SEQ ID NO: 28) displayed the highest binding capacity to all tested HNSCC cell lines (HNO97 =24.7%, HNO399=10.1%, HNO199=19.7%, HNO223=17.6%, HNO210=10.6%) which was even higher compared to the values of ¹²⁵I-SFITGv6 (HNO97=21.8%, HNO399=9.8%, HNO199=16.8%, HNO223=13.5%, HNO210=10.5%). In contrast, only moderate binding capacity was detected for SFLAP1 (4.1-12.1%) and almost none (<1%) for SFFN1, SFTNC, and SFVTN (FIG. 14A). In summary, SFLAP3 and SFITGv6 demonstrated the highest binding capacity in vitro, and thus were further investigated comparatively.

TABLE 5
Integrin αᵥβ₆ binding sequences

| | | | | | NCBI |
|---|---|---|---|---|---|
| Peptide | Peptide | Gene | | AS | reference |
| name | sequence | symbol | Protein name | length | sequence |
| SFIFN1 | GRGDSPAS | FN1 | Fibronectin | 2386 | AAI43755.1 |
| SFTNC | RRGDMSSN | TNC | Tenascin C | 2201 | NP_002151.2 |
| SFVTN | TRGDVFTM | VTN | Vitronectin | 478 | NP_000629.3 |
| SFLAP1 | RRGDLAFI | TGFB1 | Transforming | 390 | NP_000651.3 |
| | | | growth | | |
| | | | factor β1 | | |
| SFLAP3 | GRGDLGRL | TGFB3 | Transforming | 412 | NP_001316868.1 |
| | | | growth | | |
| | | | factor β3 | | |
| SFITGv6 | FRGDLMQL | N/A | N/A | N/A | N/A |

### SFLAP3 Exhibits Improved Affinity for ITGαᵥβ₆ and Excellent Serum Stability

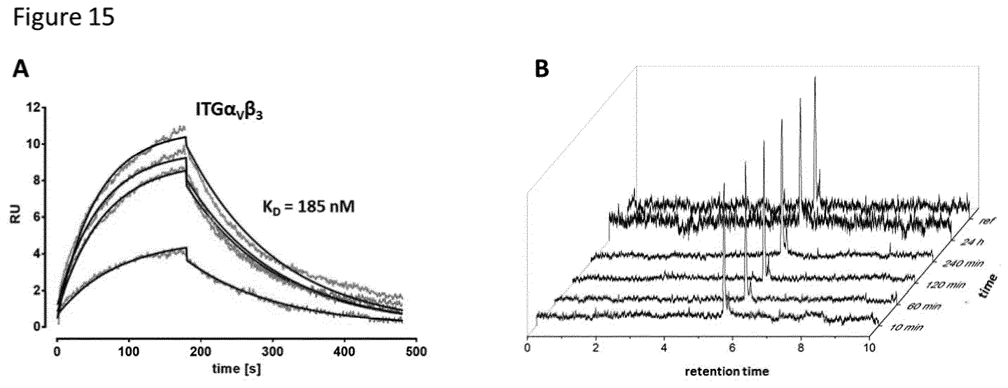

Next, competition experiments were performed to assess the specificity of the peptide binding. In all tested HNSCC cell lines, binding of both peptides ¹²⁵I-SFLAP3 and ¹²⁵I-SFITGv6 could be almost completely competed by adding 10⁻⁶ M non-radioactively labeled counterparts as competitor (FIG. 14B, C). However, SFLAP3 exhibited a higher affinity (mean IC₅₀=3.5 nM) when compared to SFITGv6 (mean IC₅₀=14.11 nM). Concordantly, the SPR spectroscopy analysis revealed a higher affinity of SFLAP3 for ITGαᵥβ₆ (K_{D}=7.4±0.9 nM) (FIG. 14D) when compared to SFITGv6 (K_{D}=14.8±26.0 nM). In contrast, we observed a 10-fold lower affinity of SFLAP3 to the αᵥβ₃ heterodimer (K_{D}=185 nM), further ensuring the ITGαᵥβ₆-specificity of SFLAP3 (FIG. 15A). Additionally, SFLAP3 revealed high proteolytic stability with no degradation over a 24 hours' time period (FIG. 15B), demonstrating the suitability of the peptide for in vivo applications.

### Comparative Evaluation of Kinetic, Internalization and Efflux

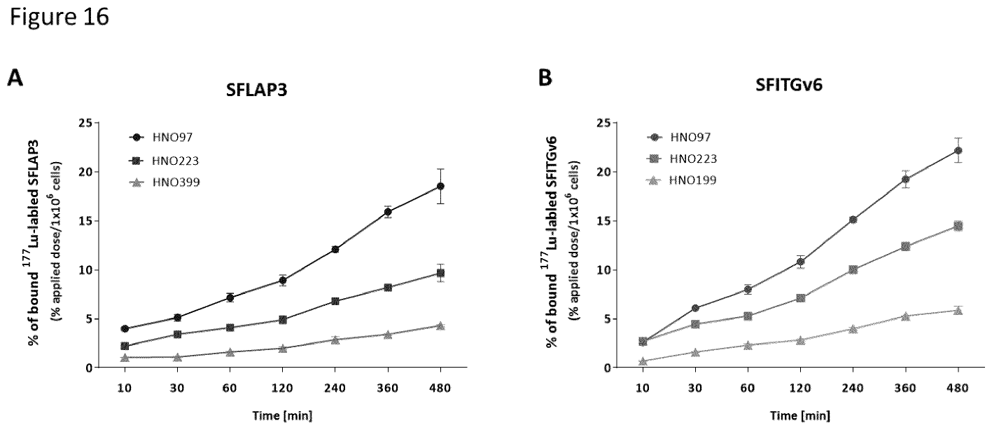

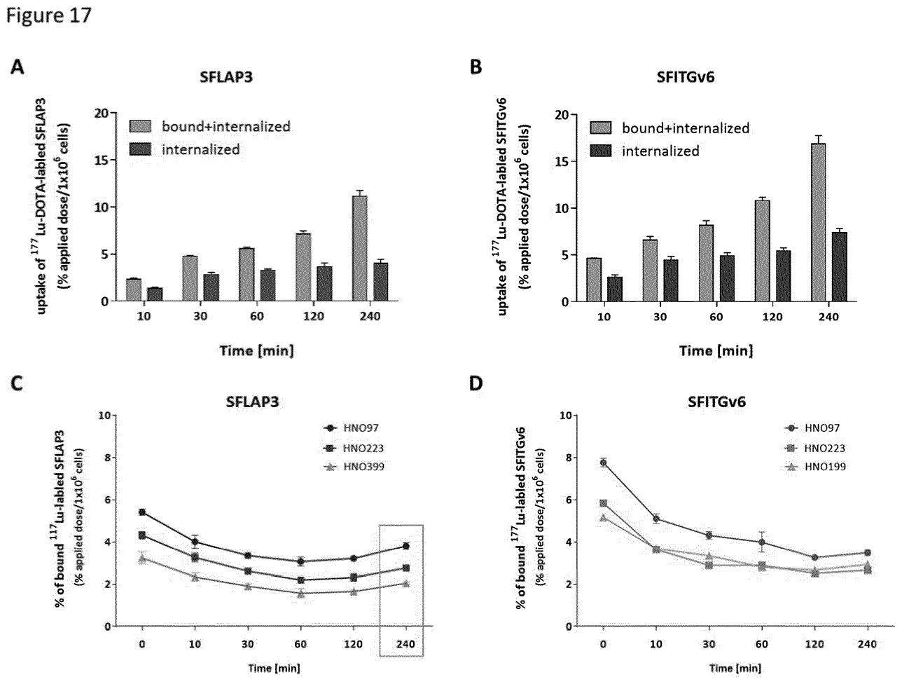

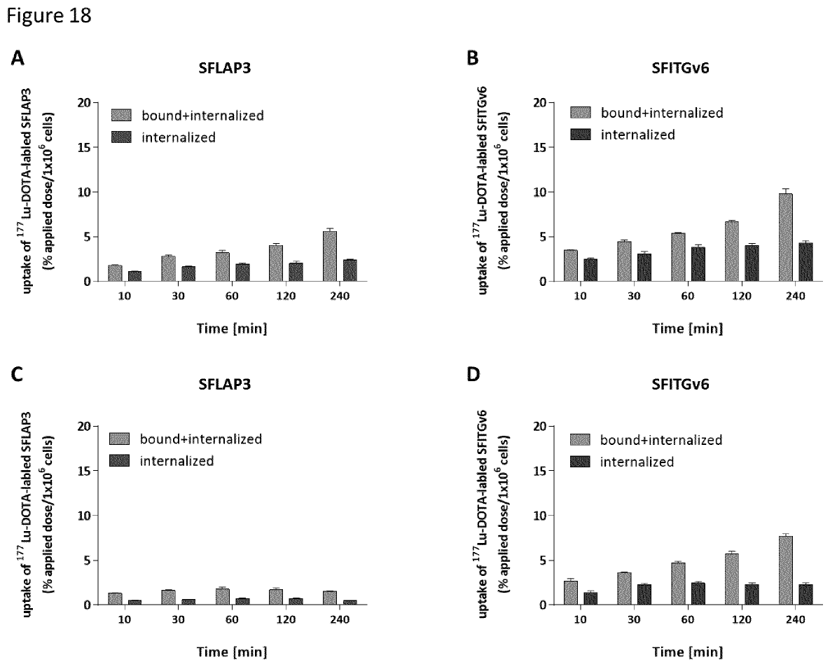

Considering time-dependent deionization of iodine 125 the determination of kinetic, internalization, and efflux were performed using ¹⁷⁷Lu-DOTA-labeled peptides. The kinetic experiment revealed a continuous increase in peptide uptake over the whole time period of measurement for both ¹⁷⁷Lu-DOTA-labeled peptides. Admittedly, ¹⁷⁷Lu-DOTA-SFLAP3 (FIG. 16A) exhibited a lower maximum binding capacity (HNO97=18.5%, HNO223=9.66%, HNO399=4.31%) when compared to ¹⁷⁷Lu-DOTA-SFITGv6 (FIG. 16B: HNO97=22.15%, HNO223=14.45%, HNO399=5.85%). Accordingly, for ¹⁷⁷Lu-DOTA-SFITGv6 we measured an increase of binding over time of up to 16.85% and a fast internalization of up to 70% of the total bound peptide, whereas for ¹⁷⁷Lu-DOTA-SFLAP3 we observed a binding capacity of 11.14% and 60% internalization within the 60 min after exposure to HNO97 (FIG. 17A, B). For both peptides comparable trends regarding time-dependent increase of peptide binding and internalization were observed after exposure to cell line HNO399 (FIG. 18A, B), and HNO223 (FIGS. 18C, D), although the amount of internalized peptide decreased (>45%). Concordantly, we observed an efflux of more than 50% of the originally accumulated ¹⁷⁷Lu-DOTA-labeled SFLAP3 and SFITGv6 within 120 min after the termination of the uptake (FIGS. 17C, D). Noteworthy, for ¹⁷⁷Lu-DOTA-SFLAP3 we observed a moderate renewed increase of binding/internalization after 240 min (FIG. 17C), which may indicate a peptide re-uptake from the culture medium.

### In Vivo Targeting Potential of SFLAP3 and SFITGv6

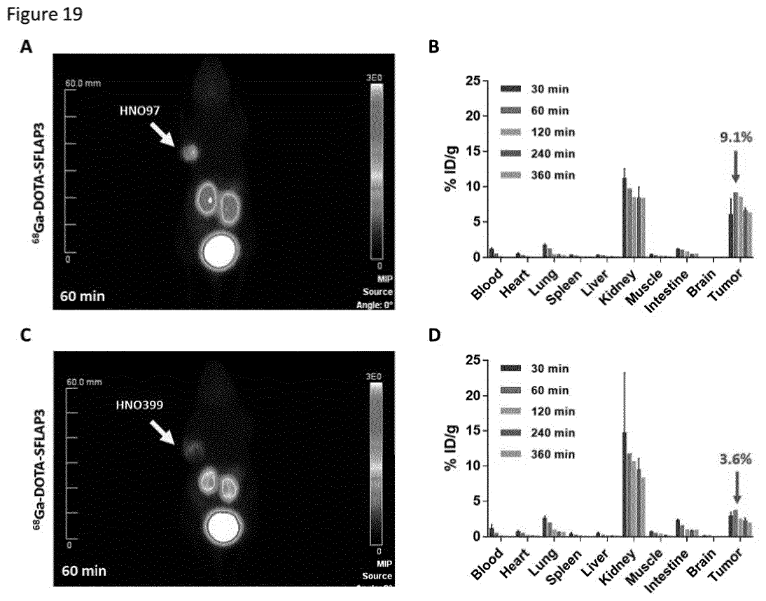

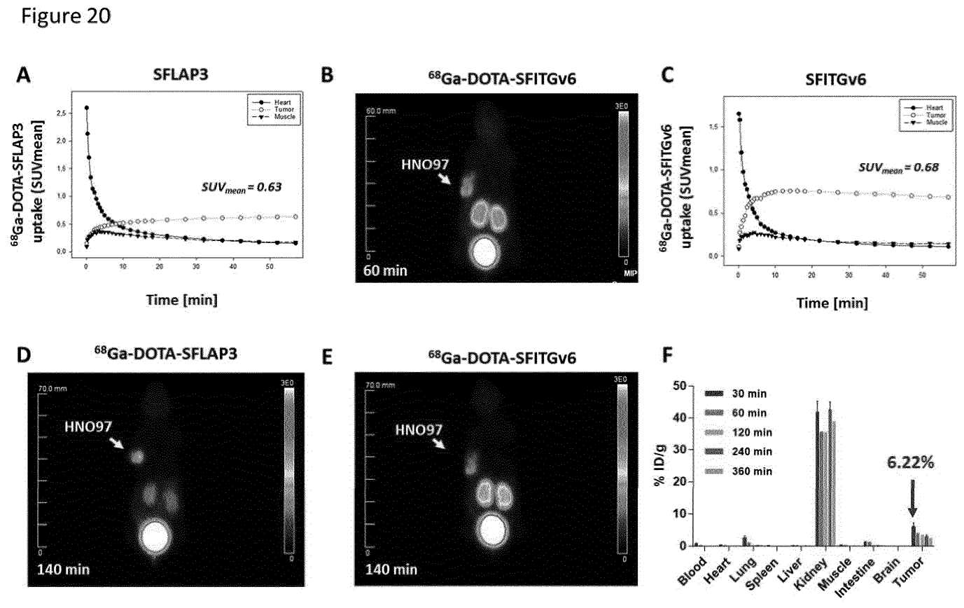

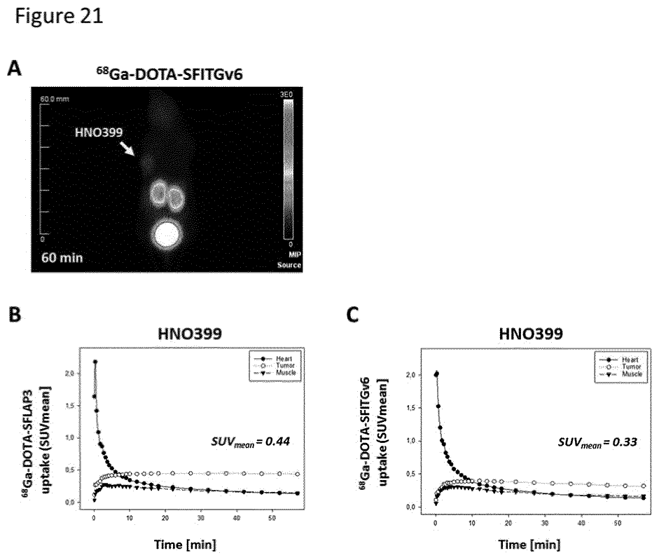

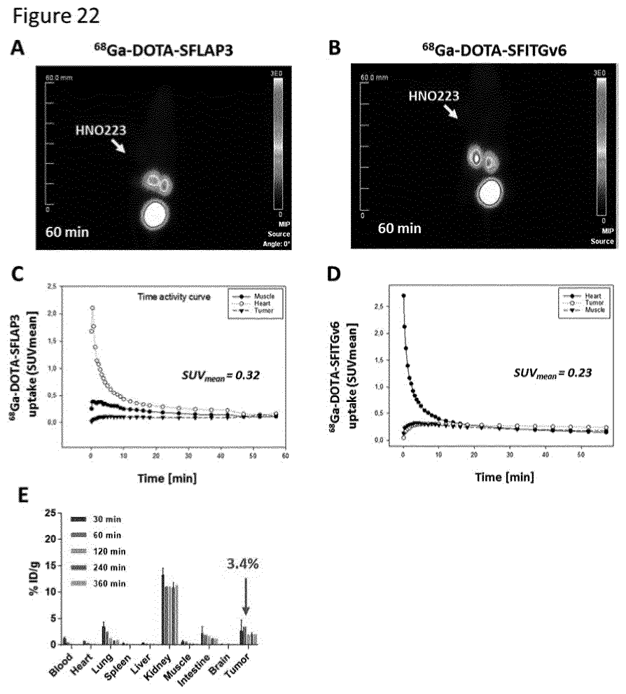

To compare the targeting properties of SFLAP3 and SFITGv6 in vivo ⁶⁸Ga-DOTA-labeled peptides were administered to HNO97-, HNO399-, or HNO223-xenografted mice and visualized by small animal PET imaging. A fast and continuous accumulation in HNO97 tumor lesions 60 min post injection was observed for both ⁶⁸Ga-DOTA-SFLAP3 [SUV mean=0.63] (FIG. 19A, 20A) and ⁶⁸Ga-DOTA-SFITGv6 [SUVmean=0.68] (FIG. 20B, C), which maintained for at least 140 min (FIG. 20D, E). Non-specific peptide activity cleared quickly from the blood resulting in a low background and images with excellent tumor-to-background ratios. Additionally, in vivo peptide biodistribution to the tumor lesions and individual organs (e.g liver, brain, and kidney) was evaluated. For ¹⁷⁷Lu-DOTA-SFLAP3 administered to HNO97-xenografts revealed a maximum peptide accumulation in the tumor after 60 min of 9.1±1.2% ID/g, followed by a decrease to 5.8±1.44% ID/g 360 min post injection (FIG. 19B). In contrast, for ¹⁷⁷Lu-DOTA-SFITGv6 the maximal uptake (6.22±1% ID/g) was measured already 30 min post injection and decreased to 2.43±0.51% ID/g after 360 min (FIG. 20F). Small animal PET imaging of HNO399 (FIG. 19C, 21A) and HNO223 (FIG. 22A, B) xenografts revealed less accumulation of ⁶⁸Ga-DOTA-SFLAP3 [HNO399: SUVmean=0.44; HNO223: SUVmean=0.32] (FIG. 21B, 22C) as well as of ⁶⁸Ga-DOTA-SFITGv6 [HNO399: SUVmean=0.33; HNO223: SUVmean=0.23] (FIG. 21C, 22D). In line with the small animal PET imaging results, the biodistribution experiments of revealed a maximum accumulation of SFLAP3 in the tumor lesion 60 min post injection of 3.6% ID/g (FIG. 19D) and 3.4% ID/g (FIG. 22E) in HNO399- and HNO233-xenografted animals, respectively. This was followed by a decrease to almost 2% measured 360 min after injection (FIG. 19D, 22E). For all experiments, less than 1% ID/g was detected 120 min after injection of the radiotracer in the blood and tissues of the xenograft mice accounting for tumor-to-tissue ratios predominantly below one (Tables 6-8). However, we observed constantly high peptide retention (up to 14% ID/g) in the kidneys, which is a common phenomenon.

TABLE 6
Tumor-to-tissue ratio of ¹⁷⁷Lu-DOTA-SFLAP3 in HNO79 xenografts

| Tumor-to-Tissue | 30 min | 60 min | 120 min | 240 min | 360 min |
|---|---|---|---|---|---|
| Tumor-to-Blood | 5.31 ± 2.24 | 18.90 ± 1.21 | 91.83 ± 1.18 | 295.73 ± 0.42 | 372.64 ± 1.44 |
| Tumor-to-Heart | 12.24 ± 2.23 | 42.48 ± 1.18 | 91.60 ± 1.18 | 122.33 ± 0.42 | 108.04 ± 1.44 |
| Tumor-to-Lung | 3.46 ± 2.24 | 7.79 ± 1.21 | 19.90 ± 1.18 | 20.20 ± 0.43 | 28.04 ± 1.44 |
| Tumar-to-Spleen | 19.76 ± 2.23 | 54.35 ± 1.18 | 104.09 ± 1.18 | 107.55 ± 0.42 | 73.46 ± 1.44 |
| Tumor-to-Liver | 18.65 ± 2.23 | 39.61 ± 1.18 | 68.58 ± 1.18 | 67.69 ± 0.42 | 46.45 ± 1.44 |
| Tumor-to-Kidney | 0.54 ± 2.60 | 0.95 ± 1.45 | 1.01 ± 1.32 | 0.79 ± 1.64 | 0.69 ± 1.63 |
| Tumor-to-Muscle | 15.09 ± 2.23 | 33.60 ± 1.18 | 55.07 ± 1.18 | 72.50 ± 0.42 | 69.98 ± 1.44 |
| Tumor-to-Intestine | 5.22 ± 2.23 | 8.71 ± 1.18 | 11.04 ± 1.19 | 16.40 ± 0.42 | 14.39 ± 1.44 |
| Tumor-to-Brain | 952.79 ± 2.23 | 464.29 ± 1.18 | 718.98 ± 1.18 | 676.81 ± 0.42 | 461.25 ± 1.44 |

TABLE 7
Tumor-to-tissue ratio of ¹⁷⁷Lu-DOTA-SFLAP3 in HNO399 xenografts

| Tumor-to-Tissue | 30 min | 60 min | 120 min | 240 min | 360 min |
|---|---|---|---|---|---|
| Tumor-to-Blood | 1.12 ± 0.78 | 7.97 ± 0.68 | 17.99 ± 0.92 | 34.78 ± 0.47 | 59.72 ± 0.32 |
| Tumor-to-Heart | 4.66 ± 0.6 | 8.61 ± 0.69 | 14.35 ± 0.92 | 21.40 ± 0.47 | 21.77 ± 0.32 |
| Tumor-to-Lung | 1.14 ± 0.64 | 1.90 ± 0.79 | 2.74 ± 0.93 | 3.94 ± 0.48 | 3.66 ± 0.33 |
| Tumor-to-Spleen | 8.60 ± 0.59 | 17.40 ± 0.67 | 20.63 ± 0.92 | 37.34 ± 0.47 | 35.91 ± 0.32 |
| Tumor-to-Liver | 7.02 ± 0.57 | 15.91 ± 0.66 | 18.55 ± 0.92 | 18.11 ± 0.47 | 17.87 ± 0.32 |
| Tumor-to-Kidney | 0.20 ± 8.55 | 0.31 ± 2.71 | 0.23 ± 3.29 | 0.23 ± 1.69 | 0.24 ± 2.74 |
| Tumor-to-Muscle | 4.7 ± 0.55 | 8.69 ± 0.66 | 8.12 ± 0.92 | 11.58 ± 0.47 | 14.66 ± 0.32 |
| Tumor-to-Intestine | 1.28 ± 0.58 | 2.49 ± 0.70 | 2.58 ± 0.95 | 2.78 ± 0.48 | 2.98 ± 0.40 |
| Tumor-to-Brain | 27.83 ± 0.55 | 34.87 ± 0.66 | 36.34 ± 0.92 | 74.29 ± 0.47 | 61.16 ± 0.32 |

TABLE 8
Tumor-to-tissue ratio of ¹⁷⁷Lu-DOTA-SFLAP3 in HNO223 xenografts

| Tumor-to-Tissue | 30 min | 60 min | 120 min | 240 min | 360 min |
|---|---|---|---|---|---|
| Tumor-to-Blood | 2.10 ± 2.17 | 8.28 ± 0.4 | 16.53 ± 0.33 | 57.65 ± 0.35 | 103.75 ± 0.57 |
| Tumor-to-Heart | 4.36 ± 2.16 | 11.54 ± 0.38 | 11.93 ± 0.33 | 19.55 ± 0.35 | 22.36 ± 0.57 |
| Tumor-to-Lung | 0.76 ± 2.33 | 1.42 ± 0.65 | 1.47 ± 0.41 | 2.81 ± 0.35 | 2.76 ± 0.59 |
| Tumor-to-Spleen | 7.83 ± 2.15 | 24.84 ± 0.37 | 18.96 ± 0.33 | 30.28 ± 0.35 | 30.11 ± 0.57 |
| Tumor-to-Liver | 6.95 ± 2.15 | 19.07 ± 0.38 | 13.68 ± 0.33 | 19.69 ± 0.35 | 20.07 ± 0.57 |
| Tumor-to-Kidney | 0.20 ± 2.54 | 0.31 ± 0.64 | 0.17 ± 2.14 | 0.18 ± 1.04 | 0.18 ± 2.73 |
| Tumor-to-Muscle | 4.14 ± 2.16 | 5.76 ± 0.45 | 6.19 ± 0.36 | 13.33 ± 0.35 | 10.61 ± 0.57 |
| Tumor-to-Intestine | 1.23 ± 2.55 | 1.80 ± 0.73 | 1.24 ± 0.66 | 1.85 ± 0.42 | 1.94 ± 0.57 |
| Tumor-to-Brain | 22.53 ± 2.15 | 62.29 ± 0.37 | 150.45 ± 0.35 | 62.09 ± 0.35 | 57.55 ± 0.57 |

### SFITGv6 In Situ Demonstrate Better Binding Properties of Exceed Those of SFLAP3

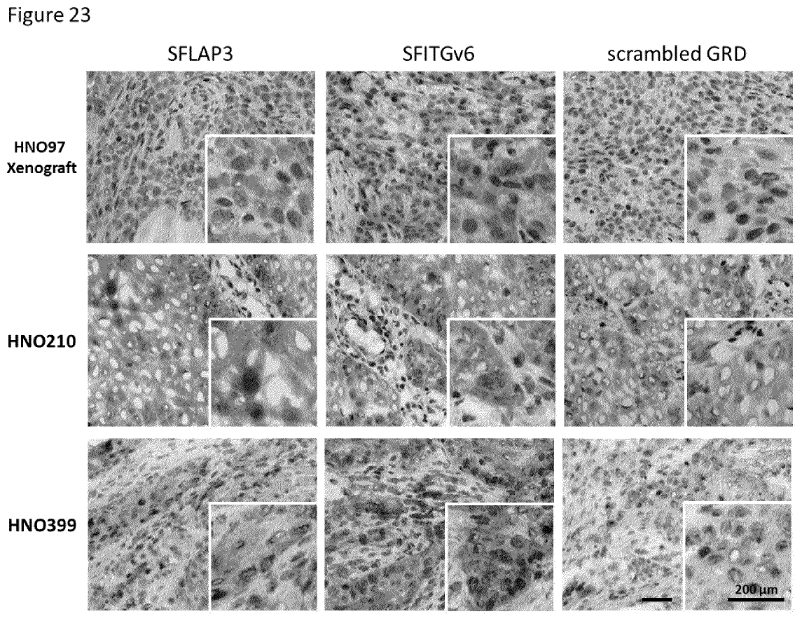

In analogy to the previous experiments, we compared the tumor cell affinity of SFLAP3 and SFITGv6 in sections of HNO97 xenograft tumors and patient-derived HNO399 and HNO210 tumors by peptide histochemical staining using biotin-labeled SFLAP3 and SFITGv6. Both peptides revealed binding to the epithelial tumor cells of the tissue sections, whereas rather low binding was observed in the stromal compartment, indicating tumor cell specificity (FIG. 23). Notably, the SFLAP3 peptide revealed a slightly fainter staining capacity. Further, we ensured the RGD-motif dependent binding affinity by applying a scrambled control peptide containing a GRD sequence instead. For the scrambled peptide we observed no binding, further signifying the specificity of the SFLAP3 peptide.

### SFLAP3 Accumulates in HNSCC Tumor Lesion and Lymph Node Metastasis

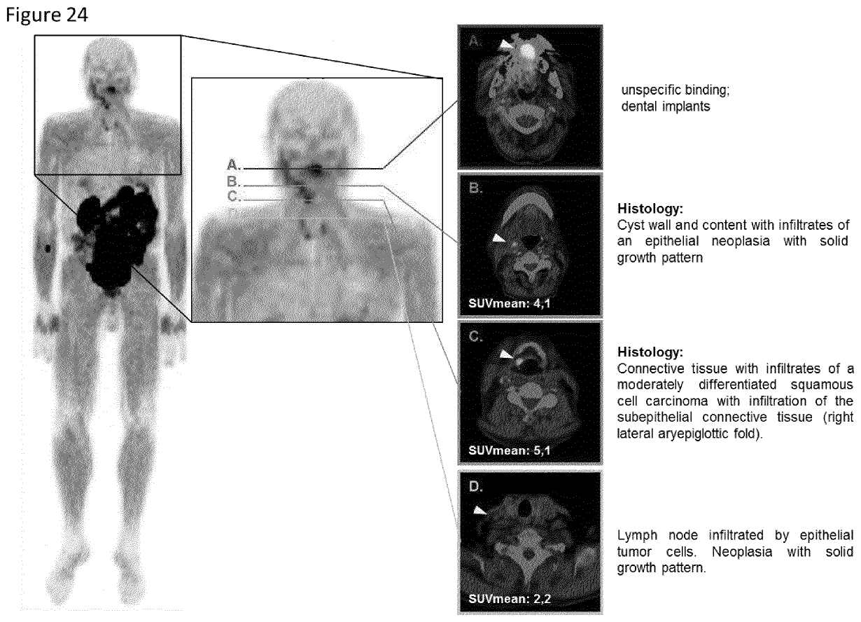

Finally, we investigated the therapeutic and/or diagnostic suitability of SFLAP3 exemplarily in one 79 year old, male carcinoma patient presented with lymph node swelling in the right cervical area assuming the diagnosis cancer of unknown primary. In order to clarify the diagnosis 322 MBq ⁶⁸Ga-DOTA-labled SFLAP3 was administered followed by a PET/CT scan (FIG. 24). After 60 minutes we observed a tumor-specific accumulation of SFLAP3 at the primary tumor lesion (right site of aryepiglottic folds) with a median SUVmax of 5.1 (Table 9). Additionally, tracer accumulation was noticed at the right cervical areal implicating a cystic lesion with epithelial neoplasia [SUVmax: 4.1]. Three days after PET/CT the respective tissues were surgically resected and a histopathological examination proved a squamous cell carcinoma and lymph node metastases of squamous cell carcinoma in Level II/III at the respective localizations.

TABLE 9
Maximum standardized uptake values (SUVmax) for PET/CT imaging
applying ⁶⁸Ga-DOTA labeled SFLAP3 to an HNSCC patient

SUVmax

| Tissue/Organ | 60 min | 180 min |
|---|---|---|
| Tumor | 5.07 | 4.64 |
| Hilar lymph nodes | 4.07 | 4.15 |
| Plexus choroideus | 1.06 | 1.57 |
| Cerebral parietal | 0.05 | 0.27 |
| Thyroid | 3.27 | 3.24 |
| Blood pool (mediast.) | 0.99 | 1.05 |
| Lung (left) | 0.36 | 0.46 |
| Lung (right) | 0.33 | 0.33 |
| Liver (left) | 0.67 | 0.76 |
| Liver (right) | 0.73 | 0.75 |
| Spleen | 0.54 | 0.8 |
| Pancreas head | 2.3 | 2.62 |
| Stomach | 12.65 | 13.4 |
| Duodenum | 6.31 | 8.91 |
| Jejunum | 10.46 | 9.2 |
| Ileum | 4.07 | 17.65 |
| Kidney (left) | 60.52 | 36.85 |
| Bight kidney | 22.43 | 26.41 |
| Colon ascendens | 9.2 | 19.37 |
| Colon descendens | 3.75 | 2.97 |
| Os ileum | 0.53 | 0.47 |
| Muscle (glut.) | 1.94 | 1.92 |

### EXAMPLE 3

### Material and Methods

### Peptide Synthesis Using Fmoc/tBU Chemistry

Peptides were synthesized using standard Fmoc/tBu solid phase peptide synthesis (SPPS) and an Applied Biosystem ABI 433A Synthesizer. Rink amid aminomethyl polystyrene resin was used as solid phase. Amino acids were thereby applied in 5 fold access. To obtain DOTA conjugated peptides, 1.2 eq. tri-tert-butyl 1,4,7,10-tetraazacyclododecane-1,4,7,10-tetraacetate was coupled to the N-terminus of the side protected peptide on solid resin. Further details as described in Example 1.

For the final oxime ligation of the trimer, the core peptide and the aminooxy-modified SFLAP3 was heated over night at pH 2-3.

### In Vitro Binding Studies

For in vitro binding Studies, 4*10⁵-6*10⁵ cells were seeded in 6 well plates and cultivated for 24 to 48 hours. At the start of the experiment the medium was replaced with 1 ml serum free medium with a definite amount of radioactive labeled peptide per well. For normal binding assays the cells were incubated for 60 min at 37° C. and afterwards washed 3 times with PBS (pH 7.4). Cells were lysed with 2 times 0.5 ml of a 0.3 M NaOH +0.2% SDS lysis-buffer. Competition assays were performed with ¹²⁵I-labeled and unlabelled (10⁻⁶ to 10⁻¹¹M) peptide and incubated at the same time for 60 min.

Cells were incubated for 10 to 240 min with ¹⁷⁷Lu-labelled peptide in internalization assays. After the washing steps, the cells were incubated with 1 ml glycine-HCl (1 M, pH 2.2) buffer for 10 min to remove surface bound peptide. Prior to the lyses the cells were washed again. Values are given as percentage of applied dose per 10⁶ cells.

The efflux was tested with ¹⁷⁷Lu-labeled peptides. After 60 min incubation the radioactive medium was removed and 1 ml fresh serum-free medium was added. After additional incubation of 60, 120 or 240 min, cell bound activity was measured.

### Animal Studies

All experiments were conducted in compliance with the German animal protection laws. 6 to 8 week old Balb c/c nude mice (Charles River Lab.) were inoculated with 5*10⁶ Capan-2 cells in 100 μl Opti-MEM medium. After 4 to 6 weeks the tumor has grown 10-15 mm in diameter. For small animal PET imaging 25 MBq of the ⁶⁸Ga-labeled peptide (2 nmol) was injected into the tail vein. Images were recorded on an Inveon small-animal PET scanner (Siemens) as described in Example 1.

For biodistribution assays 1 MBq of ¹⁷⁷Lu-labeled peptide was injected into the tail vein of each mouse. After different time points each animal was sacrificed. Peripheral blood, spleen, liver, intestine, kidney, heart, lung, brain, muscle, and tumor were removed and weighted. Radioactivity was measured in a gamma counter and expressed as injected dose per gram (% ID/g).

### PET/CT Scans of Tumor Patients

PET/CT scans of tumor patients and body scintigraphy were performed as described in Example 1.

### Results

### Binding of SFLAP3 to Pancreatic Cancer Cell-Lines

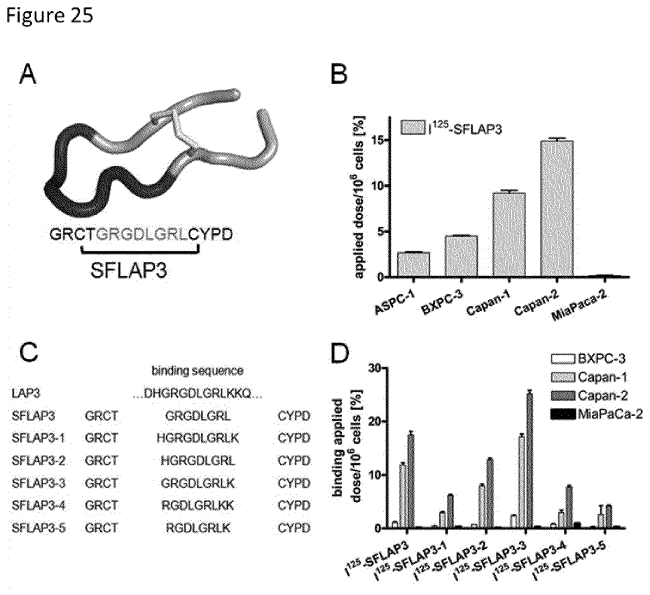

SFLAP3 is a integrin αVβ6 binding ligand in which the LAP3 binding sequence GRGDLGRL is embedded in the sunflower trypsin inhibitor-1 (SFTI) scaffold. The cysteins in the flanking amino acids GRCT and CYPD of the SFTI scaffold form disulfide bridges leading to a highly stable peptide (FIG. 25A).

Since the binding capacity of the peptide to cancer cells depends on the number of receptor molecules expressed at their surface, different cell lines were analyzed with regard to their possible use in further experiments. Uptake assays on ASPC-1, BXPC-3, Capan-1, Capan-2 and Mia PaCa-2 cells showed that Capan-2 cells had the highest ¹²⁵I-SFLAP3 binding value with 14% (FIG. 25B). FACS analysis also showed large differences in the integrin αVβ6 expression. ASPC-1 and BXPC-3 cells had a moderate (47.1% and 73.3%) while Capan-1 and Capan-2 had a high integrin αVβ6 expression (92.2% and 99.0%) (data not shown).

### Improving the Binding Ability Based on Variations of the Binding Sequence

Different variations of the SFLAP3 binding sequence GRGDLGRL were tested. In these variations the surrounding amino acids of the native LAP3 protein were added or removed and transferred into the SFTI scaffold (FIG. 25C). 5 different SFLAP3-(1-5) peptides (SEQ ID NO: 28, 30, 32, 34, 36, 38) were generated with this approach and evaluated: adding a histidine or deleting the glycine preceding the RGDLXXL motif, or appending lysine, whereas appending a lysine after this motif (SFLAP3K) (SEQ ID NO: 34) increased cellular uptake. This was shown on different pancreatic carcinoma cell lines (FIG. 25D). Labeling with iodine may lead to deiodination and diffusion of radioactivity out of the cells. Therefore, a DOTA coupled variant was made. Compared to the original ¹²⁵I-SFLAP3 peptide, ¹⁷⁷Lu-DOTA-SFLAP3 showed a decrease in cell binding with 9.5% after 60 min. ¹⁷⁷Lu-DOTA-peptides in general had lower cellular uptakes than peptides without the DOTA-chelator.

### Trimerisation of SFLAP3 Leads to an Increased Tracer Uptake

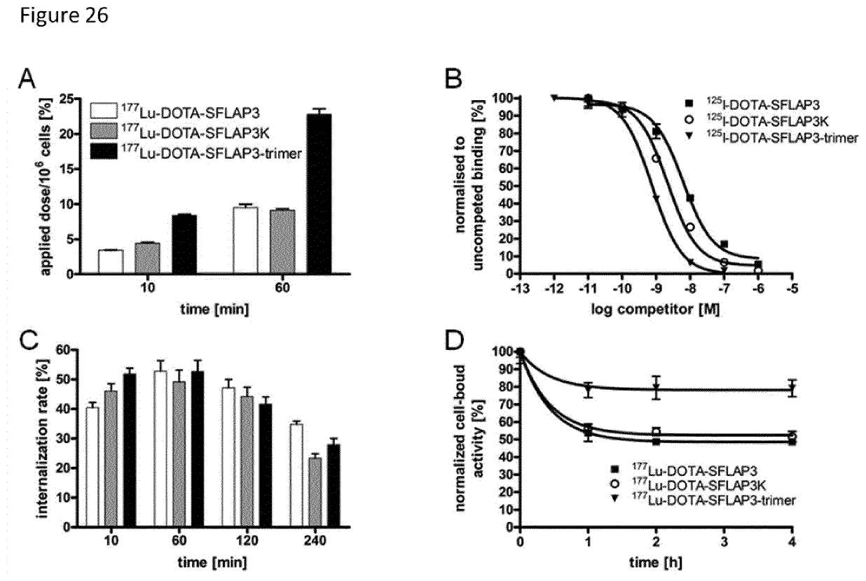

Since multimerization may lead to additive binding effects, a trimerized version of the SFLAP3 was produced using a DOTA-(GSGSK)₃ linker. The ¹⁷⁷Lu-DOTA-SFLAP3-trimer (Formula (XXVII)), ¹⁷⁷Lu-DOTA-SFLAP3K (SEQ ID NO: 34) and ¹⁷⁷Lu-DOTA-SFLAP3 (SEQ ID NO: 28) were compared regarding the cell uptake on Capan-2 cells. The addition of lysine (¹⁷⁷Lu-DOTA-SFLAP3K) did not result in an increased cellular uptake compared to the lysine-free version. This was in contrast to the DOTA free uptake results. The trimerized peptide nearly tripled the value (FIG. 26A).

### SFLAP3K and SFLAP3-Trimer Have an Increased Affinity as Compared to SFLAP3

Competition assays showed high affinities in the lower nanomolar level. Thereby only slight differences were detectable with IC₅₀ values of 6,2 nM for ¹²⁵I-DOTA-SFLAP3 and 2,1 nM for ¹²⁵1-DOTA-SFLAP3K. ¹²⁵I-DOTA-SFLAP3-trimer instead showed a high improvement in the affinity with an IC₅₀ of 0.76 nM (FIG. 26B).

With all three peptides internalization rates between 25-50% were measured (FIG. 26C). Similar internalization rates of the peptides were observed during the first two hours of incubation. However, bigger differences were detectable after 4 hours, where ¹⁷⁷Lu-DOTA-SFLAP3 had the highest internalization rate.

The efflux experiments showed that 50% of the ¹⁷⁷Lu-DOTA-SFLAP3 and the ¹⁷⁷Lu-DOTA-SFLAP3K-peptides are released into the culture medium. In contrast, the trimerized peptide showed a reduced efflux of around 20% during 4 hours following exposure of the tumor cells to the radiolabelled peptide (FIG. 26D).

### SFLAP3K and SFLAP3-Trimer Show Lower But Still Sufficient Proteolytic Stability in Human Serum

The proteolytic stability of SFLAP3 was shown for up to 24 hours in human serum. Slight degradations of SFLAP3K could be determined by radio-HPLC analysis after 4 hours of incubation. The SFLAP3-trimer degradation even started after 2 hours (data not shown). The optimal biodistribution of peptides in vivo is normally at its highest rate within the first 2 hours. Thus, the peptides should be stable for in vivo assays within the most critical time period.

### Small Animal Imaging of SFLAP3K and SFLAP3-Trimer Reveals No Improvement in Pharmacokinetics

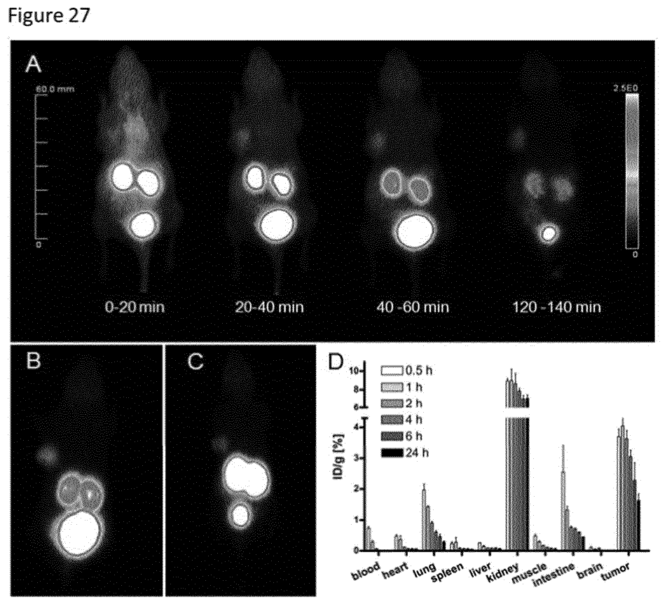

Capan-2 xenograftet balb/c nu/nu mice were injected with ⁶⁸Ga-DOTA-SFLAP3 to obtain PET images. 20 min p.i. tracer accumulation was seen in the tumor, which lasted until the end of the experiment (FIG. 27A). Except for the kidneys and the intestine, the radioactivity showed a fast clearance from normal tissues and blood. The SUV level in the kidney initially rised and then decreased nearly to the level of the tumor. The visible activity in the gut was in the area of a stuffed bowel loop.

The accumulation of ⁶⁸Ga-DOTA-SFLAP3K into the tumor was slightly better compared to the original peptide (FIG. 27B). However, the clearance of the surrounding tissue was moderately slower and the enrichment in the kidney seemed to stabilize at values which were 4 to 5 times higher than the activity in the tumor (data not shown). The ⁶⁸Ga-DOTA-SFLAP3-trimer showed no better tumor uptake (FIG. 27C). It accumulates over the complete examined time in the kidney. After 2 hours the signal is 20-30 times higher than in the tumor (data not shown) Small animal imaging revealed no benefit over the original peptide.

To obtain more precise pharmacokinetic data, Capan-2 bearing mice were injected with ¹⁷⁷Lu-DOTA-SFLAP3. At 0.5, 1, 2, 4 and 6 hours p.i. the organs and tumors were removed, weighted and the radioactivity was measured. The results are presented as injected dose/gram (ID/g). After 1 hour the tumor had the highest activity with 4% ID/g (FIG. 27D). Tumor to tissue ratios were way above 1 for all timepoints except for the kidneys (data not shown). However, the lung and intestine had by distance the highest ID/g value in comparison to the other organs.

### Diagnostic and Therapeutic Imaging in a Human Pancreas Cancer Patient

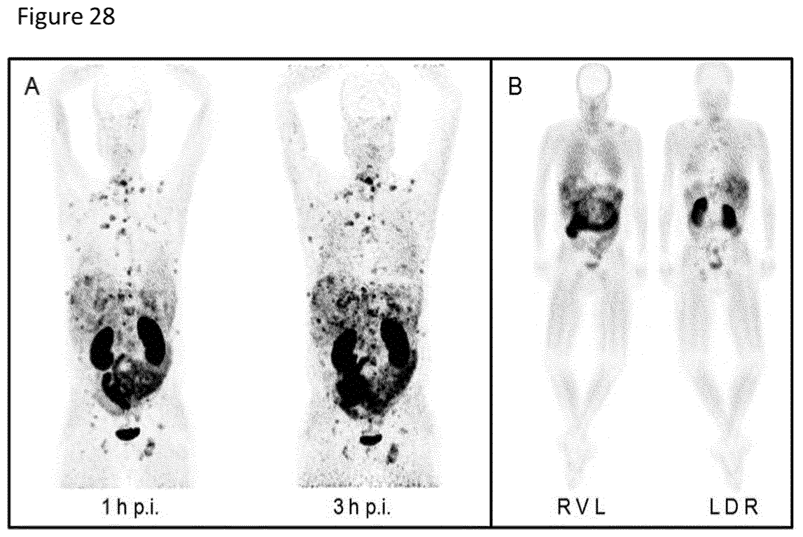

Based on the preclinical data a patient with metastasized pancreatic cancer at final stage was selected for PET/CT imaging with ⁶⁸Ga-DOTA-SFLAP3. PET imaging showed multiple metastatic sites throughout the body and especially the liver area with a high accumulation over 3 hours into the tumor lesions (FIG. 28A). Also, there was a high uptake into the kidney, the thyroid and the intestine. Since there was a shift of activity in the bowel from the 1 h image to the 3 h image, it is most likely that the tracer is located in the lumen of the intestine. The otherwise low signal background resulted in a good contrast of the metastases to the surrounding tissue.

Thereafter, the patient was treated with 6.5 GBq ¹⁷⁷Lu-DOTA-SFLAP3. Scintigraphic images 1 day after tracer administration showed an accumulation in the metastatic sites (FIG. 4B). Especially, the liver metastases showed high accumulation of the radio-ligand. On the other hand, the intestine and kidney had very high activity levels as well.

### Ovarian Cancer as Tumor Entity for the SFLAP3 Tracer

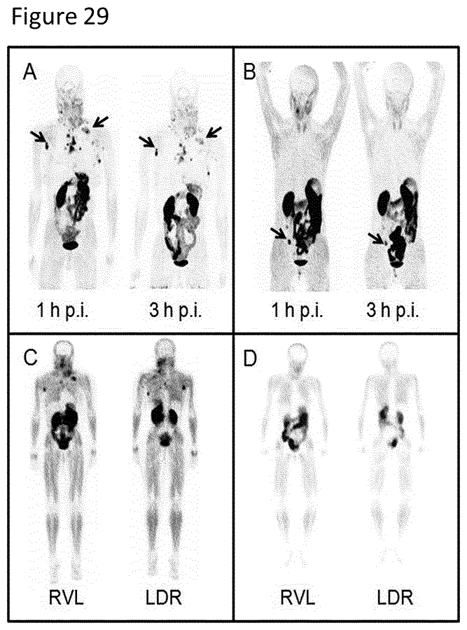

Similar to pancreatic cancer, ovarian cancer is often integrin αVβ6 positive as has been shown by immunohistochemistry studies. Tested cell lines showed low uptake of ¹²⁵I-SFLAP3 (data not shown). The cell line OV433 and OV429 with the highest binding of 5% ¹²⁵I-SFLAP3 were not tumorigenic in our mice model. Based on the positive reports of the above mentioned IHC studies PET imaging was done in 4 late stage ovarian cancer patients (2 patients shown). Metastases were clearly visible in the upper part of the upper body (FIG. 29A) or in the hip area (FIG. 29B). Out of 4 patients 2 were treated with 6 GBq and 7.4 GBq ¹⁷⁷Lu-DOTA-SFLAP3. Scintigraphic imaging showed moderate to good accumulation in the metastases 1 day after administration (FIG. 19C, D).

1. Polypeptide comprising the amino acid sequence of formula (I) or (II)
(I)
(SEQ ID NO: 1)
R₁-HN-(B₁)ₘ-C₁-(B₂)ₙ-Z₁-Z₂-X₁-R-G-D-L-X₂-X₃-L-Z₃-
(B₃)ₒ-C₂-(B₄)ₚ-COO-R₂
or
(II)
(SEQ ID NO: 2)
R₁-HN-(B₄)ₚ-C₂-(B₃)ₒ-Z₃-L-X₃-X₂-L-D-G-R-X₁-Z₂-Z₁-
(B₂)ₙ-C₁-(B₁)ₘ-COO-R₂
wherein
C₁ and C₂ form a disulfide bond;
X₁ is any amino acid;
X₂ is any amino acid;
X₃ is any amino acid;
Z₁ is any amino acid, or not present;
Z₂ is any amino acid, or not present;
Z₃ is any amino acid or not present;
B₁ is any amino acid, m is an integer selected from 0, 1, 2 or 3;
B₂ is any amino acid, n is an integer selected from 0, 1, or 2;
B₃ is any amino acid, o is an integer selected from 0, 1, 2 or 3;
B₄ is any amino acid, p is an integer selected from 0, 1, 2 or 3;
R₁ is H, a therapeutic agent, an imaging agent, a linker group or a linker group linked to a therapeutic agent, or an imaging agent; and
R₂ is H, a therapeutic agent, an imaging agent, a linker group or a linker group linked to a therapeutic agent, or an imaging agent.

2. Polypeptide of claim 1 further comprising at least one compound selected from the group consisting of an imaging agent, a therapeutic agent, a linker group or a linker group linked to a therapeutic agent or an imaging agent.

3. Polypeptide of claim 1, wherein the therapeutic agent is present at R1 and/or R2 and is selected from the group consisting of small molecules, biopharmaceuticals, and combinations thereof.

4. Polypeptide of claim 3, wherein the therapeutic agent is a small molecule, and wherein the small molecule is an anti-cancer therapeutic.

5. Polypeptide of claim 3, wherein the therapeutic agent is a biopharmaceutical, and wherein the biopharmaceutical is a protein.

6. Polypeptide of claim 1, wherein the therapeutic agent is present at R1 and/or R2 and further comprises a radioisotope.

7. Polypeptide of claim 1, wherein the imaging agent is present at R1 and/or R2 and is a polydentate chelating agent.

8. Polypeptide of claim 7, wherein the polydentate chelating agent further comprises a radioisotope.

9. Polypeptide of claim 1, wherein the imaging agent is present at R1 and/or R2 and is a molecule comprising a radioisotope selected from the group consisting of 18F, HC, and combinations thereof.

10. Polypeptide of claim 9, wherein the imaging agent is selected from the group consisting of [¹¹C]metomidate, [N-methyl-¹¹C]vorozole, [¹¹C]glucose, and combinations thereof.

11. Polypeptide of claim 1, wherein the imaging agent is present at R1 and/or R2 and is a monodentate complex-forming molecule.

12. Polypeptide of claim 11, wherein the monodentate complex-forming molecule further comprises a radioisotope.

13. Polypeptide of claim 1, wherein the linker group is present at R1 and/or R2 and is selected from the group consisting of alkyl, cycloalkyl, heteroalkyl, arylalkyl, alkoxy, aryloxy, amino, amide, silyl, alkenyl, cycloalkenyl, heteroalkenyl, alkynyl, aryl, heteroaryl, acyl, carbonyl, carboxylic acid, ether, ester, nitrile, isonitrile, sulfanyl, sulfinyl, sulfonyl, phosphino, and combinations thereof.

14. Polypeptide of claim 1 for use in the treatment of cancer or in the diagnosis and/or evaluation of cancer.

15. Polypeptide of claim 14, wherein the cancer is selected from the group consisting of head and neck squamous cell carcinoma, hypopharynx carcinoma, collangiocarcinoma, breast cancer, brain cancer, pancreatic cancer, ovarian cancer, kidney cancer, bowel cancer, stomach cancer, cervical cancer, thyroid cancer, pancreatic cancer, gastric cancer, lung cancer, colorectal cancer, liver cancer.

16. Polypeptide of claim 1, wherein:
X₁ is an amino acid selected from the group consisting of F, W, T, G, A, I, L, M, V;
X₂ is an amino acid selected from the group consisting of M, A, I, L, V, G;
X₃ is an amino acid selected from the group consisting of Q, D, R and L;
Z₁ is an amino acid selected from the group consisting of F, M, A, V, I, L, Y and W, or not present;
Z₂ is an amino acid selected from the group consisting of T, Q, S, and N, or not present;
Z₃ is any amino acid or not present;
B₁ is an amino acid selected from the group consisting of G, A, H, K and R, and m is 2;
n is 0;
o is 0; and
B₄ is an amino acid selected from the group consisting of Y, W, P, E, and D, and p is

3.

17. Polypeptide of claim 1, wherein:
X₁ is an amino acid selected from the group consisting of F and G;
X₂ is an amino acid selected from the group consisting of M and G;
X₃ is an amino acid selected from the group consisting of Q and R;
Z₁ is an amino acid selected from the group consisting of F, Y and W;
Z₂ is an amino acid selected from the group consisting of T and S;
Z₃ is any amino acid or not present;
B₁ in formula (I) is GH, GK, or GR, and B₁ in formula (II) is HG, KG, or KG;
n is 0;
o is 0; and
B₄ formula (I) is YPD, FPD, WPD, YPE, FPE or WPE and B₄ in formula (II) is DPY, DPF, DPW, EPY, EPF, or EPW.

18. Polypeptide of claim 1, wherein:
X₁ is F;
X₂ is M;
X₃ is Q;
Zᵢ is F;
Z₂ is T;
Z₃ is any amino acid or not present;
B₁ in formula (I) is GR and in formula (II) is RG, and m is 2;
n is 0;
o is 0;
B₄ is YPD in formula (I) and DPY in formula (II), and p is 3.

19. Polypeptide of claim 4, wherein the small molecule is selected from the group consisting of cyclophosphamide, methotrexate, 5-fluorouracil, mustine, vincristine, procarbazine, prednisolone, doxorubicin, bleomycin, vinblastine, dacarbazine, vincristine, etoposide, cisplatin, epirubicin, capecitabine, folinic acid, oxaliplatin, and combinations thereof.


SEQUENCE LISTING (43 sequences)

SEQ ID NO: 1 - protein, 17 aa, Artificial Sequence
  Synthetic peptide
  MISC_FEATURE (1)..(1): is 0, 1, 2 or 3 amino acids
  DISULFID (2)..(16)
  MISC_FEATURE (3)..(3): is 0, 1, or 2 amino acids
  MISC_FEATURE (4)..(4): is any amino acid, preferably selected from F, M, A, V, I, L, Y, and W
  MISC_FEATURE (5)..(5): is any amino acid, preferably an amino acid selected from T, Q, S, and N
  MISC_FEATURE (6)..(6): is any amino acid, preferably selected from F, W, T, G, A, I, L, M, and V
  MISC_FEATURE (11)..(11): is any amino acid, preferably selected from M, A, I, L, V, and G
  MISC_FEATURE (12)..(12): is any amino acid, preferably selected from Q, D, R, and L
  MISC_FEATURE (14)..(14): is any amino acid or not present
  MISC_FEATURE (15)..(15): is 0, 1, 2, or 3 amino acids
  MISC_FEATURE (17)..(17): is 0, 1, 2, or 3 amino acids
XCXXXXRGDLXXLXXCX

SEQ ID NO: 2 - protein, 17 aa, Artificial Sequence
  Synthetic peptide
  MISC_FEATURE (1)..(1): is 0, 1, 2, or 3 amino acids
  DISULFID (2)..(16)
  MISC_FEATURE (3)..(3): is 0, 1, 2, or 3 amino acids
  MISC_FEATURE (4)..(4): is any amino acid or not present
  MISC_FEATURE (6)..(6): is any amino acid, preferably selected from Q, D, R, and L
  MISC_FEATURE (7)..(7): is any amino acid, preferably selected from M, A, I, L, V, and G
  MISC_FEATURE (12)..(12): is any amino acid, preferably selected from F, W, T, G, A, I, L, M, and V
  MISC_FEATURE (13)..(13): is any amino acid, preferably selected from T, Q, S, and N
  MISC_FEATURE (14)..(14): is any amino acid, preferably selected from F, M, A, V, I, L, Y, and W
  MISC_FEATURE (15)..(15): is 0, 1, or 2 amino acids
  MISC_FEATURE (17)..(17): is 0, 1, 2, or 3 amino acids
XCXXLXXLDGRXXXXCX

SEQ ID NO: 3 - protein, 16 aa, Artificial Sequence
  Synthetic peptide
  DISULFID (3)..(13)
GRCTFRGDLMQLCYPD

SEQ ID NO: 4 - protein, 16 aa, Artificial Sequence
  Synthetic peptide
  DISULFID (4)..(14)
DPYCLQMLDGRFTCGR

SEQ ID NO: 5 - protein, 16 aa, Artificial Sequence
  Synthetic peptide
  DISULFID (3)..(13)
GRCTGRGDLGRLCYPD

SEQ ID NO: 6 - protein, 16 aa, Artificial Sequence
  Synthetic peptide
  DISULFID (4)..(14)
DPYCLRGLDGRGTCGR

SEQ ID NO: 7 - protein, 16 aa, Artificial Sequence
  Synthetic peptide
  DISULFID (3)..(13)
GRCTFRGDLMQLCYPD

SEQ ID NO: 8 - protein, 16 aa, Artificial Sequence
  Synthetic peptide
  DISULFID (4)..(14)
DPYCLQMLDGRFTCGR

SEQ ID NO: 9 - protein, 16 aa, Artificial Sequence
  Synthetic peptide
  DISULFID (3)..(13)
GRCTGRGDLGRLCYPD

SEQ ID NO: 10 - protein, 16 aa, Artificial Sequence
  Synthetic peptide
  DISULFID (4)..(14)
DPYCLRGLDGRGTCGR

SEQ ID NO: 11 - protein, 8 aa, Artificial Sequence
  Synthetic peptide
FRGDLMQL

SEQ ID NO: 12 - DNA, 65 bp, Artificial Sequence
  Synthetic oligonucleotide
  misc_feature (30)..(53): n is a, c, g, or t
ttactcgctccatgggcggcaggtgtactnnnnnnnnnnnnnnnnnnnnnnnntgttatcccgat

SEQ ID NO: 13 - DNA, 43 bp, Artificial Sequence
  Synthetic oligonucleotide
ataatcttgcggccgcaccgccacctgctgcatcgggataaca

SEQ ID NO: 14 - protein, 8 aa, Artificial Sequence
  Synthetic peptide
WWKFMWFQ

SEQ ID NO: 15 - protein, 8 aa, Artificial Sequence
  Synthetic peptide
FRGDKMQL

SEQ ID NO: 16 - protein, 8 aa, Artificial Sequence
  Synthetic peptide
RAWWSHWH

SEQ ID NO: 17 - protein, 8 aa, Artificial Sequence
  Synthetic peptide
HWFWSKWW

SEQ ID NO: 18 - protein, 8 aa, Artificial Sequence
  Synthetic peptide
WRWASWFW

SEQ ID NO: 19 - protein, 8 aa, Artificial Sequence
  Synthetic peptide
WHQNIHSP

SEQ ID NO: 20 - protein, 8 aa, Artificial Sequence
  Synthetic peptide
FDRGKMQL

SEQ ID NO: 21 - protein, 8 aa, Artificial Sequence
  Synthetic peptide
FRGDAMQA

SEQ ID NO: 22 - protein, 17 aa, Artificial Sequence
  Synthetic peptide
  MISC_FEATURE (1)..(1): is o, 1, 2, or 3 amino acids
  DISULFID (2)..(16)
  MISC_FEATURE (3)..(3): is 0, 1, or 2 amino acids
  MISC_FEATURE (4)..(4): is any amino acid, preferably selected from T, Q, S, and N, or not present
  MISC_FEATURE (5)..(5): is any amino acid, preferably selected from F, M, A, V, I, L, H, Y, and W, or not present
  MISC_FEATURE (6)..(6): is any amino acid, preferably selected from F, W, T, G, A, I, L, M, V, or not present
  MISC_FEATURE (11)..(11): is any amino acid, preferably selected from M, A, I, L, V, and G
  MISC_FEATURE (12)..(12): is any amino acid, preferably selected from Q, D, R, and L
  MISC_FEATURE (14)..(14): is any amino acid or not present
  MISC_FEATURE (15)..(15): is 0, 1, 2, or 3 amino acids
  MISC_FEATURE (17)..(17): is 0, 1, 2, or 3 amino acids
XCXXXXRGDLXXLXXCX

SEQ ID NO: 23 - protein, 17 aa, Artificial Sequence
  Synthetic peptide
  MISC_FEATURE (1)..(1): is 0, 1, 2, or 3 amino acids
  DISULFID (2)..(16)
  MISC_FEATURE (3)..(3): is 0, 1, 2, or 3 amino acids
  MISC_FEATURE (4)..(4): is any amino acid or not present
  MISC_FEATURE (6)..(6): is any amino acid, preferably selected from Q, D, R, and L
  MISC_FEATURE (7)..(7): is any amino acid, preferably selected from M, A, I, L, V, and G
  MISC_FEATURE (12)..(12): is any amino acid, preferably selected from F, W, T, G, A, I, L,M, and V, or not present
  MISC_FEATURE (13)..(13): is any amino acid, preferably selected from F, M, A, V, I, L, H, Y, and W, or not present
  MISC_FEATURE (14)..(14): is any amino acid, preferably selected from T, Q, S, and N, or not present
  MISC_FEATURE (15)..(15): is 0, 1, or 2 amino acids
  MISC_FEATURE (17)..(17): 0, 1, 2, or 3 amino acids
XCXXLXXLDGRXXXXCX

SEQ ID NO: 24 - protein, 16 aa, Artificial Sequence
  Synthetic peptide
  MISC_FEATURE (1)..(1): is 0, 1, 2, or 3 amino acids
  DISULFID (2)..(15)
  MISC_FEATURE (3)..(3): is any amino acid, preferably selected from T and S, or not present
  MISC_FEATURE (4)..(4): is any amino acid, preferably selected from F, H, Y, and W, or not present
  MISC_FEATURE (5)..(5): is any amino acid, preferably selected from F and G, or not present
  MISC_FEATURE (10)..(10): is any amino acid, preferably selected from M and G
  MISC_FEATURE (11)..(11): is any amino acid, preferably selected from Q and R
  MISC_FEATURE (13)..(13): is any amino acid or not present
  MISC_FEATURE (14)..(14): is  0, 1, 2, or 3 amino acids
  MISC_FEATURE (16)..(16): is  0, 1, 2, or 3 amino acids
XCXXXRGDLXXLXXCX

SEQ ID NO: 25 - protein, 16 aa, Artificial Sequence
  Synthetic peptide
  MISC_FEATURE (1)..(1): is 0, 1, 2, or 3 amino acids
  DISULFID (2)..(15)
  MISC_FEATURE (3)..(3): is 0, 1, 2, or 3 amino acids
  MISC_FEATURE (4)..(4): is any amino acid or not present
  MISC_FEATURE (6)..(6): is any amino acid, preferably selected from Q and R
  MISC_FEATURE (7)..(7): is any amino acid, preferably selected from M and G
  MISC_FEATURE (12)..(12): is any amino acid, preferably selected from  F and G, or not present
  MISC_FEATURE (13)..(13): is any amino acid, preferably selected from  F, H, Y, and W, or not present
  MISC_FEATURE (14)..(14): is any amino acid, preferably selected from  T and S, or not present
  MISC_FEATURE (16)..(16): is 0, 1, 2, or 3 amino acids
XCXXLXXLDGRXXXCX

SEQ ID NO: 26 - protein, 18 aa, Artificial Sequence
  Synthetic peptide
  DISULFID (3)..(15)
  MISC_FEATURE (5)..(5): is any amino acid, preferably selected from F, H, Y, and W, or not present
  MISC_FEATURE (6)..(6): is any amino acid, preferably selected from F and G, or not present
  MISC_FEATURE (14)..(14): is 0, 1, 2, or 3 amino acids
GRCTXXRGDLGRLXCYPD

SEQ ID NO: 27 - protein, 18 aa, Artificial Sequence
  Synthetic peptide
  DISULFID (4)..(16)
  MISC_FEATURE (5)..(5): is 0, 1, 2, or 3 amino acids
  MISC_FEATURE (13)..(13): is any amino acid, preferably selected from F and G, or not present
  MISC_FEATURE (14)..(14): is any amino acid, preferably selected from F, H, Y, and W, or not present
DPYCXLRGLDGRXXTCRG

SEQ ID NO: 28 - protein, 16 aa, Artificial Sequence
  Synthetic peptide
  DISULFID (3)..(13)
GRCTGRGDLGRLCYPD

SEQ ID NO: 29 - protein, 16 aa, Artificial Sequence
  Synthetic peptide
  DISULFID (4)..(14)
DPYCLRGLDGRGTCRG

SEQ ID NO: 30 - protein, 18 aa, Artificial Sequence
  Artificial Sequence
  DISULFID (3)..(15)
GRCTHGRGDLGRLKCYPD

SEQ ID NO: 31 - protein, 18 aa, Artificial Sequence
  Synthetic peptide
  DISULFID (4)..(16)
DPYCKLRGLDGRGHTCRG

SEQ ID NO: 32 - protein, 17 aa, Artificial Sequence
  Synthetic peptide
  DISULFID (3)..(14)
GRCTHGRGDLGRLCYPD

SEQ ID NO: 33 - protein, 17 aa, Artificial Sequence
  Synthetic peptide
  DISULFID (4)..(15)
DPYCLRGLDGRGHTCRG

SEQ ID NO: 34 - protein, 17 aa, Artificial Sequence
  Synthetic peptide
  DISULFID (3)..(14)
GRCTGRGDLGRLKCYPD

SEQ ID NO: 35 - protein, 17 aa, Artificial Sequence
  Synthetic peptide
  DISULFID (4)..(15)
DPYCKLRGLDGRGTCRG

SEQ ID NO: 36 - protein, 17 aa, Artificial Sequence
  Synthetic peptide
  DISULFID (3)..(14)
GRCTRGDLGRLKKCYPD

SEQ ID NO: 37 - protein, 17 aa, Artificial Sequence
  Synthetic peptide
  DISULFID (4)..(15)
DPYCKKLRGLDGRTCRG

SEQ ID NO: 38 - protein, 16 aa, Artificial Sequence
  Synthetic peptide
  DISULFID (3)..(13)
GRCTRGDLGRLKCYPD

SEQ ID NO: 39 - protein, 16 aa, Artificial Sequence
  Synthetic peptide
  DNA_BIND (4)..(14)
DPYCKLRGLDGRTCRG

SEQ ID NO: 40 - protein, 5 aa, Artificial Sequence
  Synthetic peptide
GSGSK

SEQ ID NO: 41 - protein, 58 aa, Artificial Sequence
  Synthetic peptide
GRCTGRGDLGRLCYPDGSGSKGRCTGRGDLGRLCYPDGSGSKGRCTGRGDLGRLCYPD

SEQ ID NO: 42 - protein, 7 aa, Artificial Sequence
  Generalization of an integrin binding motive
  misc_feature (5)..(6): Xaa can be any naturally occurring amino acid
RGDKXXL

SEQ ID NO: 43 - protein, 7 aa, Artificial Sequence
  Generalization of an integrin binding motive
  misc_feature (5)..(6): Xaa can be any naturally occurring amino acid
RGDLXXL